SEQUENCE LISTING (116 sequences)

SEQ ID NO: 1 - protein, 185 aa, Homo sapiens
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 2 - DNA, 555 bp, Homo sapiens
aaagagatcacaaacgccctggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctacgacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggac

SEQ ID NO: 3 - protein, 185 aa, Artificial Sequence
  CD2 Extracellular domain with D111H mutation
KEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 4 - DNA, 555 bp, Artificial Sequence
  CD2 extracellular domain witha  D111H mutation
aaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggac

SEQ ID NO: 5 - protein, 24 aa, Homo sapiens
MSFPCKFVASFLLIFNVSSKGAVS

SEQ ID NO: 6 - DNA, 72 bp, Homo sapiens
atgagcttcccttgcaagttcgtggccagcttcctgctgatcttcaacgtgtcctctaagggcgccgtgtcc

SEQ ID NO: 7 - protein, 21 aa, Homo sapiens
MALPVTALLLPLALLLHAARP

SEQ ID NO: 8 - DNA, 63 bp, Homo sapiens
atggctctgcctgtgacagctctgctgctgcctctggctctgcttctgcatgccgccagacct

SEQ ID NO: 9 - protein, 26 aa, Homo sapiens
IYLIIGICGGGSLLMVFVALLVFYIT

SEQ ID NO: 10 - DNA, 78 bp, Homo sapiens
atctacctgatcatcggcatctgtggcggcggatccctgctgatggtgtttgtggctctgctggtgttctacatcacc

SEQ ID NO: 11 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 336 bp, Homo sapiens
agagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 13 - protein, 116 aa, Homo sapiens
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 14 - DNA, 348 bp, Homo sapiens
aagcggaagaagcagcggagcagacggaacgacgaggaactggaaacacgggcccatagagtggccaccgaggaaagaggcagaaagccccaccagattccagccagcacaccccagaatcctgccacctctcaacaccctccacctccacctggacacagatctcaggccccatctcacagacctccaccacctggtcatcgggtgcagcaccagcctcagaaaagacctcctgctcctagcggcacacaggtgcaccagcaaaaaggacctccactgcctcggcctagagtgcagcctaaacctcctcatggcgccgctgagaacagcctgtctccaagcagcaac

SEQ ID NO: 15 - protein, 41 aa, Homo sapiens
RSKRSRLLHSDYMNMTPRRPGPTRKHYQPYAPPRDFAAYRS

SEQ ID NO: 16 - DNA, 123 bp, Homo sapiens
agaagcaagcggagccggctgctgcacagcgactacatgaacatgacccctagacggcccggacctaccagaaagcactaccagccttacgctcctcctagagacttcgccgcctaccggtcc

SEQ ID NO: 17 - protein, 42 aa, Homo sapiens
KRGRKKLLYIFKQPFMRPVQTTQEEDGCSCRFPEEEEGGCEL

SEQ ID NO: 18 - DNA, 126 bp, homo sapiens
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 19 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 20 - DNA, 585 bp, Homo sapiens
aagaagcggatcaagcccatcgtgtggcccagcctgcctgaccacaagaaaaccctggaacacctgtgcaagaagccccggaagaacctgaatgtgtccttcaatcccgagagcttcctggactgccagatccacagagtggacgacatccaggccagagatgaggtggaaggctttctgcaggacacctttccacagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagcagcagaagcctggattgcagagagagcggaaagaacggccctcatgtgtatcaggacctgctgctgagcctgggcaccaccaattctacactgcctccaccattcagcctgcagagcggcatcctgacactgaaccctgttgctcagggccagccaatcctgacaagcctgggctccaatcaagaagaggcctacgtcaccatgtccagcttctaccagaaccag

SEQ ID NO: 21 - protein, 39 aa, Homo sapiens
KSRQTPPLASVEMEAMEALPVTWGTSSRDEDLENCSHHL

SEQ ID NO: 22 - DNA, 117 bp, Homo sapiens
aagagccggcagacacctcctctggccagcgtggaaatggaagccatggaagctctgcctgtgacctggggcacaagcagcagagatgaggacctggaaaactgcagccaccacctg

SEQ ID NO: 23 - protein, 285 aa, Homo sapiens
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 24 - DNA, 855 bp, Homo sapiens
agcctgaaaacacacccactctggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagggatagaccttacggcctggtgtctatcgacaccgtgaccgtgctggatgccgagggaccttgtacatggccttgcagctgcgaggacgatggctaccctgctctggatctggacgcaggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggccagc

SEQ ID NO: 25 - protein, 38 aa, Homo sapiens
CWLTKKKYSSSVHDPNGEYMFMRAVNTAKKSRLTDVTL

SEQ ID NO: 26 - DNA, 114 bp, Homo sapiens
tgttggctgaccaagaaaaagtacagcagcagcgtgcacgaccccaacggcgagtacatgttcatgagagccgtgaacaccgccaagaagtccagactgaccgacgtgaccctg

SEQ ID NO: 27 - protein, 48 aa, Homo sapiens
QRRKYRSNKGESPVEPAEPCHYSCPREEEGSTIPIQEDYRKPEPACSP

SEQ ID NO: 28 - DNA, 144 bp, Homo sapiens
cagcggcggaagtacagaagcaacaagggcgagagccccgtggaacctgccgagccttgtcactacagctgccccagagaggaagagggcagcacaatccccatccaagaggactacagaaagcccgagcctgcctgctctccc

SEQ ID NO: 29 - protein, 42 aa, Homo sapiens
ALYLLRRDQRLPPDAHKPPGGGSFRTPIQEEQADAHSTLAKI

SEQ ID NO: 30 - DNA, 126 bp, Homo sapiens
gccctgtacctgctgcggcgggatcaaagattgcctcctgacgctcacaagcctccaggcggaggcagctttagaacccctatccaagaggaacaggctgacgcccacagcaccctggccaagatc

SEQ ID NO: 31 - protein, 58 aa, Homo sapiens
QLGLHIWQLRSQCMWPRETQLLLEVPPSTEDARSCQFPEEERGERSAEEKGRLGDLWV

SEQ ID NO: 32 - DNA, 180 bp, Homo sapiens
cagctgggactgcacatctggcagctgagaagccagtgcatgtggcccagagagacacagctgctgctggaagtgcctcctagcaccgaggatgccagaagctgtcagttccccgaggaagagagaggcgagagatccgccgaggaaaaaggcagactgggcgacctgtgggtccgagtg

SEQ ID NO: 33 - protein, 463 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1389 bp, Homo sapiens
atgagcttcccttgcaagttcgtggccagcttcctgctgatcttcaacgtgtcctctaagggcgccgtgtccaaagagatcacaaacgccctggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctacgacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggaagcctgctgatggtgtttgtggctctgctggtgttctacatcaccaagcggaagaagcagcggagcagacggaacgacgaggaactggaaacacgggcccatagagtggccaccgaggaaagaggcagaaagccccaccagattccagccagcacaccccagaatcctgccacctctcaacaccctccacctccacctggacacagatctcaggccccatctcacagacctccaccacctggtcatcgggtgcagcaccagcctcagaaaagacctcctgctcctagcggcacacaggtgcaccagcaaaaaggacctccactgcctcggcctagagtgcagcctaaacctcctcatggcgccgctgagaacagcctgtctccaagcagcaacagagtgaagttcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 35 - protein, 463 aa, Artificial Sequence
  CSR 01 CD2.DH.z
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 1389 bp, Artificial Sequence
  CSR 01 CD2.DH.z
atgagcttcccttgcaagttcgtggccagcttcctgctgatcttcaacgtgtcctctaagggcgccgtgtccaaagagatcacaaacgccctggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggaagcctgctgatggtgtttgtggctctgctggtgttctacatcaccaagcggaagaagcagcggagcagacggaacgacgaggaactggaaacacgggcccatagagtggccaccgaggaaagaggcagaaagccccaccagattccagccagcacaccccagaatcctgccacctctcaacaccctccacctccacctggacacagatctcaggccccatctcacagacctccaccacctggtcatcgggtgcagcaccagcctcagaaaagacctcctgctcctagcggcacacaggtgcaccagcaaaaaggacctccactgcctcggcctagagtgcagcctaaacctcctcatggcgccgctgagaacagcctgtctccaagcagcaacagagtgaagttcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 37 - protein, 460 aa, Artificial Sequence
  CD2.8.DH.z polypeptide
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 1380 bp, Artificial Sequence
  CD2.8.DH.z polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccaagcggaagaagcagcggagcagacggaacgacgaggaactggaaacacgggcccatagagtggccaccgaggaaagaggcagaaagccccaccagattccagccagcacaccccagaatcctgccacctctcaacaccctccacctccacctggacacagatctcaggccccatctcacagacctccaccacctggtcatcgggtgcagcaccagcctcagaaaagacctcctgctcctagcggcacacaggtgcaccagcaaaaaggacctccactgcctcggcctagagtgcagcctaaacctcctcatggcgccgctgagaacagcctgtctccaagcagcaacagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 39 - protein, 388 aa, Artificial Sequence
  CD2.DH.28.z polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1164 bp, Artificial Sequence
  CD2.DH.28.z polynudeotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccagaagcaagcggagccggctgctgcacagcgactacatgaacatgacccctagacggcccggacctaccagaaagcactaccagccttacgctcctcctagagacttcgccgcctaccggtccagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 41 - protein, 385 aa, Artificial Sequence
  CD2.8.DH.28z polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1155 bp, Artificial Sequence
  CD2.8.DH.28z polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccagaagcaagcggagccggctgctgcacagcgactacatgaacatgacccctagacggcccggacctaccagaaagcactaccagccttacgctcctcctagagacttcgccgcctaccggtccagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 43 - protein, 389 aa, Artificial Sequence
  CD2.DH.BBz polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 1167 bp, Artificial Sequence
  CD2.DH.BBz polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 45 - protein, 386 aa, Artificial Sequence
  CD2.8.DH.BBz polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 1158 bp, Artificial Sequence
  CD2.8.DH.BBz polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 47 - protein, 542 aa, Artificial Sequence
  CD2.DH.7z polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 1626 bp, Artificial Sequence
  CD2.DH.7z polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccaagaagcggatcaagcccatcgtgtggcccagcctgcctgaccacaagaaaaccctggaacacctgtgcaagaagccccggaagaacctgaatgtgtccttcaatcccgagagcttcctggactgccagatccacagagtggacgacatccaggccagagatgaggtggaaggctttctgcaggacacctttccacagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagcagcagaagcctggattgcagagagagcggaaagaacggccctcatgtgtatcaggacctgctgctgagcctgggcaccaccaattctacactgcctccaccattcagcctgcagagcggcatcctgacactgaaccctgttgctcagggccagccaatcctgacaagcctgggctccaatcaagaagaggcctacgtcaccatgtccagcttctaccagaaccagagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 49 - protein, 539 aa, Artificial Sequence
  CD2.8.DH.7z polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - DNA, 1617 bp, Artificial Sequence
  CD2.8.DH.7z polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccaagaagcggatcaagcccatcgtgtggcccagcctgcctgaccacaagaaaaccctggaacacctgtgcaagaagccccggaagaacctgaatgtgtccttcaatcccgagagcttcctggactgccagatccacagagtggacgacatccaggccagagatgaggtggaaggctttctgcaggacacctttccacagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagcagcagaagcctggattgcagagagagcggaaagaacggccctcatgtgtatcaggacctgctgctgagcctgggcaccaccaattctacactgcctccaccattcagcctgcagagcggcatcctgacactgaaccctgttgctcagggccagccaatcctgacaagcctgggctccaatcaagaagaggcctacgtcaccatgtccagcttctaccagaaccagagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 51 - protein, 386 aa, Artificial Sequence
  CD2.DH.15z polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKSRQTPPLASVEMEAMEALPVTWGTSSRDEDLENCSHHLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 1158 bp, Artificial Sequence
  CD2.DH.15z polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccaagagccggcagacacctcctctggccagcgtggaaatggaagccatggaagctctgcctgtgacctggggcacaagcagcagagatgaggacctggaaaactgcagccaccacctgagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 53 - protein, 383 aa, Artificial Sequence
  CD2.8.DH.15z polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKSRQTPPLASVEMEAMEALPVTWGTSSRDEDLENCSHHLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 1149 bp, Artificial Sequence
  CD2.8.DH.15z polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccaagagccggcagacacctcctctggccagcgtggaaatggaagccatggaagctctgcctgtgacctggggcacaagcagcagagatgaggacctggaaaactgcagccaccacctgagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 55 - protein, 632 aa, Artificial Sequence
  CD2.DH.21z polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQASRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 1896 bp, Artificial Sequence
  CD2.DH.21z polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccagcctgaaaacacacccactctggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagggatagaccttacggcctggtgtctatcgacaccgtgaccgtgctggatgccgagggaccttgtacatggccttgcagctgcgaggacgatggctaccctgctctggatctggacgcaggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggccagcagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 57 - protein, 629 aa, Artificial Sequence
  CD2.8.DH.21z polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQASRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1887 bp, Artificial Sequence
  CD2.8.DH.21z polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccagcctgaaaacacacccactctggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagggatagaccttacggcctggtgtctatcgacaccgtgaccgtgctggatgccgagggaccttgtacatggccttgcagctgcgaggacgatggctaccctgctctggatctggacgcaggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggccagcagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 59 - protein, 385 aa, Artificial Sequence
  CD2.DH.Iz polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITCWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 1155 bp, Artificial Sequence
  CD2.DH.Iz polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcacctgttggctgaccaagaaaaagtacagcagcagcgtgcacgaccccaacggcgagtacatgttcatgagagccgtgaacaccgccaagaagtccagactgaccgacgtgaccctgagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 61 - protein, 382 aa, Artificial Sequence
  CD2.8.DH.Iz polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITCWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1146 bp, Artificial Sequence
  CD2.8.DH.Iz polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcacctgttggctgaccaagaaaaagtacagcagcagcgtgcacgaccccaacggcgagtacatgttcatgagagccgtgaacaccgccaagaagtccagactgaccgacgtgaccctgagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 63 - protein, 395 aa, Artificial Sequence
  CD2.DH.27z polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITQRRKYRSNKGESPVEPAEPCHYSCPREEEGSTIPIQEDYRKPEPACSPRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 1185 bp, Artificial Sequence
  CD2.DH.27z polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcacccagcggcggaagtacagaagcaacaagggcgagagccccgtggaacctgccgagccttgtcactacagctgccccagagaggaagagggcagcacaatccccatccaagaggactacagaaagcccgagcctgcctgctctcccagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 65 - protein, 392 aa, Artificial Sequence
  CD2.8.DH.27z polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITQRRKYRSNKGESPVEPAEPCHYSCPREEEGSTIPIQEDYRKPEPACSPRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 1176 bp, Artificial Sequence
  CD2.8.DH.27z polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcacccagcggcggaagtacagaagcaacaagggcgagagccccgtggaacctgccgagccttgtcactacagctgccccagagaggaagagggcagcacaatccccatccaagaggactacagaaagcccgagcctgcctgctctcccagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 67 - protein, 389 aa, Artificial Sequence
  CD2.DH.Oxz polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1167 bp, Artificial Sequence
  CD2.DH.Oxz polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccgccctgtacctgctgcggcgggatcaaagattgcctcctgacgctcacaagcctccaggcggaggcagctttagaacccctatccaagaggaacaggctgacgcccacagcaccctggccaagatcagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 69 - protein, 386 aa, Artificial Sequence
  CD2.8.DH.Oxz polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - DNA, 1158 bp, Artificial Sequence
  CD2.8.DH.Oxz polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcaccgccctgtacctgctgcggcgggatcaaagattgcctcctgacgctcacaagcctccaggcggaggcagctttagaacccctatccaagaggaacaggctgacgcccacagcaccctggccaagatcagagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 71 - protein, 405 aa, Artificial Sequence
  CD2.DH.Gz polypeptide sequence
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITQLGLHIWQLRSQCMWPRETQLLLEVPPSTEDARSCQFPEEERGERSAEEKGRLGDLWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - DNA, 1215 bp, Artificial Sequence
  CD2.DH.Gz polynucleotide sequence
atgagcttcccttgcaagttcgtggccagcttcctgctgatcttcaacgtgtcctctaagggcgccgtgtccaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcacacagctgggactgcacatctggcagctgagaagccagtgcatgtggcccagagagacacagctgctgctggaagtgcctcctagcaccgaggatgccagaagctgtcagttccccgaggaagagagaggcgagagatccgccgaggaaaaaggcagactgggcgacctgtgggtccgagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 73 - protein, 402 aa, Artificial Sequence
  CD2.8.DH.Gz polypeptide sequence
MALPVTALLLPLALLLHAARPKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITQLGLHIWQLRSQCMWPRETQLLLEVPPSTEDARSCQFPEEERGERSAEEKGRLGDLWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1206 bp, Artificial Sequence
  CD2.8.DH.Gz polynucleotide sequence
atggctctgcctgtgacagctctgctgctgcctctggctctgcttctgcatgccgccagacctaaagaaatcacgaatgcattggaaacctggggagccctcggccaggatattaacctggacatccccagcttccagatgagcgacgacatcgatgacatcaagtgggagaaaaccagcgacaagaagaagatcgcccagttccggaaagagaaagagacattcaaagagaaggacacctacaagctgttcaagaacggcaccctgaagatcaagcacctgaaaaccgacgaccaggacatctataaggtgtccatctaccacaccaagggcaagaacgtgctggaaaagatcttcgacctcaagatccaagagcgggtgtccaagcctaagatcagctggacctgcatcaacaccacactgacctgcgaagtgatgaacggcacagaccccgagctgaacctgtaccaggatggcaaacacctgaagctgagccagcgcgtgatcacccacaagtggacaacaagcctgagcgccaagttcaagtgcaccgccggaaacaaagtgtctaaagagtccagcgtcgagcccgtgtcttgccctgaaaaaggactggacatctacctgatcatcggcatctgtggcggcggatccctgctgatggtgtttgtggctctgctggtgttctacatcacacagctgggactgcacatctggcagctgagaagccagtgcatgtggcccagagagacacagctgctgctggaagtgcctcctagcaccgaggatgccagaagctgtcagttccccgaggaagagagaggcgagagatccgccgaggaaaaaggcagactgggcgacctgtgggtccgagtgaaattcagccgcagcgccgatgctcctgcctataagcagggacagaaccagctgtacaacgagctgaatctggggcgcagagaagagtacgatgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 75-78 - skipped (4 entries)

SEQ ID NO: 79 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 80 - protein, 1053 aa, Artificial Sequence
  dSaCas9
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 81 - protein, 1368 aa, Staphylococcus pyogenes
  misc_feature (1)..(1): Xaa can be any naturally occurring amino acid
XDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 82 - protein, 1368 aa, Artificial Sequence
  dCas9 synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 83 - protein, 199 aa, Artificial Sequence
  Clo051
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 84 - protein, 1591 aa, Artificial Sequence
  dCas9-Clo051 fusion protein
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 85 - DNA, 4776 bp, Artificial Sequence
  dCas9-Clo051 fusion protein
atggcaccaaagaagaaaagaaaagtggagggcatcaagtcaaacatcagcctgctgaaagacgaactgcggggacagattagtcacatcagtcacgagtacctgtcactgattgatctggccttcgacagcaagcagaatagactgtttgagatgaaagtgctggaactgctggtcaacgagtatggcttcaagggcagacatctgggcgggtctaggaaacctgacggcatcgtgtacagtaccacactggaagacaacttcggaatcattgtcgataccaaggcttattccgagggctactctctgccaattagtcaggcagatgagatggaaaggtacgtgcgcgaaaactcaaatagggacgaggaagtcaaccccaataagtggtgggagaatttcagcgaggaagtgaagaaatactacttcgtctttatctcaggcagcttcaaagggaagtttgaggaacagctgcggagactgtccatgactaccggggtgaacggatctgctgtcaacgtggtcaatctgctgctgggcgcagaaaagatcaggtccggggagatgacaattgaggaactggaacgcgccatgttcaacaattctgagtttatcctgaagtatggaggcgggggaagcgataagaaatactccatcggactggccattggcaccaattccgtgggctgggctgtcatcacagacgagtacaaggtgccaagcaagaagttcaaggtcctggggaacaccgatcgccacagtatcaagaaaaatctgattggagccctgctgttcgactcaggcgagactgctgaagcaacccgactgaagcggactgctaggcgccgatatacccggagaaaaaatcggatctgctacctgcaggaaattttcagcaacgagatggccaaggtggacgatagtttctttcaccgcctggaggaatcattcctggtggaggaagataagaaacacgagcggcatcccatctttggcaacattgtggacgaagtcgcttatcacgagaagtaccctactatctatcatctgaggaagaaactggtggactccaccgataaggcagacctgcgcctgatctatctggccctggctcacatgatcaagttccgggggcattttctgatcgagggagatctgaaccctgacaattctgatgtggacaagctgttcatccagctggtccagacatacaatcagctgtttgaggaaaacccaattaatgcctcaggcgtggacgcaaaggccatcctgagcgccagactgtccaaatctaggcgcctggaaaacctgatcgctcagctgccaggagagaagaaaaacggcctgtttgggaatctgattgcactgtccctgggcctgacacccaacttcaagtctaattttgatctggccgaggacgctaagctgcagctgtccaaagacacttatgacgatgacctggataacctgctggctcagatcggcgatcagtacgcagacctgttcctggccgctaagaatctgagtgacgccatcctgctgtcagatattctgcgcgtgaacacagagattactaaggccccactgagtgcttcaatgatcaaaagatatgacgagcaccatcaggatctgaccctgctgaaggctctggtgaggcagcagctgcccgagaaatacaaggaaatcttctttgatcagagcaagaatggatacgccggctatattgacggcggggcttcccaggaggagttctacaagttcatcaagcccattctggaaaagatggacggcaccgaggaactgctggtgaagctgaatcgggaggacctgctgagaaaacagaggacatttgataacggaagcatccctcaccagattcatctgggcgaactgcacgccatcctgcgacggcaggaggacttctacccatttctgaaggataaccgcgagaaaatcgaaaagatcctgaccttcagaatcccctactatgtggggcctctggcacggggaaatagtagatttgcctggatgacaagaaagtcagaggaaactatcaccccctggaacttcgaggaagtggtcgataaaggcgctagcgcacagtccttcattgaaaggatgacaaattttgacaagaacctgccaaatgagaaggtgctgcccaaacacagcctgctgtacgaatatttcacagtgtataacgagctgactaaagtgaagtacgtcaccgaagggatgcgcaagcccgcattcctgtccggagagcagaagaaagccatcgtggacctgctgtttaagacaaatcggaaagtgactgtcaaacagctgaaggaagactatttcaagaaaattgagtgtttcgattcagtggaaatcagcggcgtcgaggacaggtttaacgcctccctggggacctaccacgatctgctgaagatcatcaaggataaggacttcctggacaacgaggaaaatgaggacatcctggaggacattgtgctgacactgactctgtttgaggatcgcgaaatgatcgaggaacgactgaagacttatgcccatctgttcgatgacaaagtgatgaagcagctgaaaagaaggcgctacaccggatggggacgcctgagccgaaaactgatcaatgggattagagacaagcagagcggaaaaactatcctggactttctgaagtccgatggcttcgccaacaggaacttcatgcagctgattcacgatgactctctgaccttcaaggaggacatccagaaagcacaggtgtctggccagggggacagtctgcacgagcatatcgcaaacctggccggcagccccgccatcaagaaagggattctgcagaccgtgaaggtggtggacgaactggtcaaggtcatgggacgacacaaacctgagaacatcgtgattgagatggcccgcgaaaatcagacaactcagaagggccagaaaaacagtcgagaacggatgaagagaatcgaggaaggcatcaaggagctggggtcacagatcctgaaggagcatcctgtggaaaacactcagctgcagaatgagaaactgtatctgtactatctgcagaatggacgggatatgtacgtggaccaggagctggatattaacagactgagtgattatgacgtggatgccatcgtccctcagagcttcctgaaggatgactccattgacaacaaggtgctgaccaggtccgacaagaaccgcggcaaatcagataatgtgccaagcgaggaagtggtcaagaaaatgaagaactactggaggcagctgctgaatgccaagctgatcacacagcggaaatttgataacctgactaaggcagaaagaggaggcctgtctgagctggacaaggccggcttcatcaagcggcagctggtggagacaagacagatcactaagcacgtcgctcagattctggatagcagaatgaacacaaagtacgatgaaaacgacaagctgatcagggaggtgaaagtcattactctgaaatccaagctggtgtctgactttagaaaggatttccagttttataaagtcagggagatcaacaactaccaccatgctcatgacgcatacctgaacgcagtggtcgggaccgccctgattaagaaataccccaagctggagtccgagttcgtgtacggagactataaagtgtacgatgtccggaagatgatcgccaaatctgagcaggaaattggcaaggccaccgctaagtatttcttttacagtaacatcatgaatttctttaagaccgaaatcacactggcaaatggggagatcagaaaaaggcctctgattgagaccaacggggagacaggagaaatcgtgtgggacaagggaagggattttgctaccgtgcgcaaagtcctgtccatgccccaagtgaatattgtcaagaaaactgaagtgcagaccgggggattctctaaggagagtattctgcctaagcgaaactctgataaactgatcgcccggaagaaagactgggaccccaagaagtatggcgggttcgactctccaacagtggcttacagtgtcctggtggtcgcaaaggtggaaaaggggaagtccaagaaactgaagtctgtcaaagagctgctgggaatcactattatggaacgcagctccttcgagaagaatcctatcgattttctggaagccaagggctataaagaggtgaagaaagacctgatcattaagctgccaaaatactcactgtttgagctggaaaacggacgaaagcgaatgctggcaagcgccggagaactgcagaagggcaatgagctggccctgccctccaaatacgtgaacttcctgtatctggctagccactacgagaaactgaaggggtcccctgaggataacgaacagaagcagctgtttgtggagcagcacaaacattatctggacgagatcattgaacagatttcagagttcagcaagagagtgatcctggctgacgcaaatctggataaagtcctgagcgcatacaacaagcaccgagacaaaccaatccgggagcaggccgaaaatatcattcatctgttcaccctgacaaacctgggcgcccctgcagccttcaagtattttgacaccacaatcgatcggaagagatacacttctaccaaagaggtgctggatgctaccctgatccaccagagtattaccggcctgtatgagacacgcatcgacctgtcacagctgggaggcgatgggagccccaagaaaaagcggaaggtgtctagttaa

SEQ ID NO: 86 - protein, 1588 aa, Artificial Sequence
  dCas9-Clo051 fusion protein
MPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 87 - DNA, 4767 bp, Artificial Sequence
  dCas9-Clo051 fusion protein
atgcctaagaagaagcggaaggtggaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatacggtggcggtggatccgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctga

SEQ ID NO: 88 - protein, 187 aa, Artificial Sequence
  DHFR mutein enzyme
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 89 - DNA, 561 bp, Artificial Sequence
  DHFR mutein enzyme
atggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgac

SEQ ID NO: 90 - protein, 18 aa, Artificial Sequence
  T2A peptide
EGRGSLLTCGDVEENPGP

SEQ ID NO: 91 - protein, 21 aa, Artificial Sequence
  GSG-T2A peptide
GSGEGRGSLLTCGDVEENPGP

SEQ ID NO: 92 - DNA, 63 bp, Artificial Sequence
  GSG-T2A polynucleotide
ggatctggagagggaaggggaagcctgctgacctgtggagacgtggaggaaaacccaggacca

SEQ ID NO: 93 - protein, 20 aa, Artificial Sequence
  E2A peptide
QCTNYALLKLAGDVESNPGP

SEQ ID NO: 94 - protein, 23 aa, Artificial Sequence
  GSG-E2A peptide
GSGQCTNYALLKLAGDVESNPGP

SEQ ID NO: 95 - protein, 22 aa, Artificial Sequence
  F2A peptide
VKQTLNFDLLKLAGDVESNPGP

SEQ ID NO: 96 - protein, 25 aa, Artificial Sequence
  GSG-F2A peptide
GSGVKQTLNFDLLKLAGDVESNPGP

SEQ ID NO: 97 - protein, 19 aa, Artificial Sequence
  P2A peptide
ATNFSLLKQAGDVEENPGP

SEQ ID NO: 98 - protein, 22 aa, Artificial Sequence
  GSG-P2A peptide
GSGATNFSLLKQAGDVEENPGP

SEQ ID NO: 99 - protein, 594 aa, Artificial Sequence
  PB transposase
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 100 - protein, 594 aa, Artificial Sequence
  SPB transposase
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 101 - protein, 340 aa, Artificial Sequence
  Sleeping Beauty transposase
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 102 - protein, 340 aa, Artificial Sequence
  hyperactive Sleeping Beauty (SB100X) transposase
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 103 - DNA, 5296 bp, Artificial Sequence
  Helraiser transposon
tcctatataataaaagagaaacatgcaaattgaccatccctccgctacgctcaagccacgcccaccagccaatcagaagtgactatgcaaattaacccaacaaagatggcagttaaatttgcatacgcaggtgtcaagcgccccaggaggcaacggcggccgcgggctcccaggaccttcgctggccccgggaggcgaggccggccgcgcctagccacacccgcgggctcccgggaccttcgccagcagagagcagagcgggagagcgggcggagagcgggaggtttggaggacttggcagagcaggaggccgctggacatagagcagagcgagagagagggtggcttggagggcgtggctccctctgtcaccccagcttcctcatcacagctgtggaaactgacagcagggaggaggaagtcccacccccacagaatcagccagaatcagccgttggtcagacagctctcagcggcctgacagccaggactctcattcacctgcatctcagaccgtgacagtagagaggtgggactatgtctaaagaacaactgttgatacaacgtagctctgcagccgaaagatgccggcgttatcgacagaaaatgtctgcagagcaacgtgcgtctgatcttgaaagaaggcggcgcctgcaacagaatgtatctgaagagcagctactggaaaaacgtcgctctgaagccgaaaaacagcggcgtcatcgacagaaaatgtctaaagaccaacgtgcctttgaagttgaaagaaggcggtggcgacgacagaatatgtctagagaacagtcatcaacaagtactaccaataccggtaggaactgccttctcagcaaaaatggagtacatgaggatgcaattctcgaacatagttgtggtggaatgactgttcgatgtgaattttgcctatcactaaatttctctgatgaaaaaccatccgatgggaaatttactcgatgttgtagcaaagggaaagtctgtccaaatgatatacattttccagattacccggcatatttaaaaagattaatgacaaacgaagattctgacagtaaaaatttcatggaaaatattcgttccataaatagttcttttgcttttgcttccatgggtgcaaatattgcatcgccatcaggatatgggccatactgttttagaatacacggacaagtttatcaccgtactggaactttacatccttcggatggtgtttctcggaagtttgctcaactctatattttggatacagccgaagctacaagtaaaagattagcaatgccagaaaaccagggctgctcagaaagactcatgatcaacatcaacaacctcatgcatgaaataaatgaattaacaaaatcgtacaagatgctacatgaggtagaaaaggaagcccaatctgaagcagcagcaaaaggtattgctcccacagaagtaacaatggcgattaaatacgatcgtaacagtgacccaggtagatataattctccccgtgtaaccgaggttgctgtcatattcagaaacgaagatggagaacctccttttgaaagggacttgctcattcattgtaaaccagatcccaataatccaaatgccactaaaatgaaacaaatcagtatcctgtttcctacattagatgcaatgacatatcctattctttttccacatggtgaaaaaggctggggaacagatattgcattaagactcagagacaacagtgtaatcgacaataatactagacaaaatgtaaggacacgagtcacacaaatgcagtattatggatttcatctctctgtgcgggacacgttcaatcctattttaaatgcaggaaaattaactcaacagtttattgtggattcatattcaaaaatggaggccaatcggataaatttcatcaaagcaaaccaatctaagttgagagttgaaaaatatagtggtttgatggattatctcaaatctagatctgaaaatgacaatgtgccgattggtaaaatgataatacttccatcatcttttgagggtagtcccagaaatatgcagcagcgatatcaggatgctatggcaattgtaacgaagtatggcaagcccgatttattcataaccatgacatgcaaccccaaatgggcagatattacaaacaatttacaacgctggcaaaaagttgaaaacagacctgacttggtagccagagtttttaatattaagctgaatgctcttttaaatgatatatgtaaattccatttatttggcaaagtaatagctaaaattcatgtcattgaatttcagaaacgcggactgcctcacgctcacatattattgatattagatagtgagtccaaattacgttcagaagatgacattgaccgtatagttaaggcagaaattccagatgaagaccagtgtcctcgactttttcaaattgtaaaatcaaatatggtacatggaccatgtggaatacaaaatccaaatagtccatgtatggaaaatggaaaatgttcaaagggatatccaaaagaatttcaaaatgcgaccattggaaatattgatggatatcccaaatacaaacgaagatctggtagcaccatgtctattggaaataaagttgtcgataacacttggattgtcccttataacccgtatttgtgccttaaatataactgtcatataaatgttgaagtctgtgcatcaattaaaagtgtcaaatatttatttaaatacatctataaagggcacgattgtgcaaatattcaaatttctgaaaaaaatattatcaatcatgacgaagtacaggacttcattgactccaggtatgtgagcgctcctgaggctgtttggagactttttgcaatgcgaatgcatgaccaatctcatgcaatcacaagattagctattcatttgccaaatgatcagaatttgtattttcataccgatgattttgctgaagttttagatagggctaaaaggcataactcgactttgatggcttggttcttattgaatagagaagattctgatgcacgtaattattattattgggagattccacagcattatgtgtttaataattctttgtggacaaaacgccgaaagggtgggaataaagtattaggtagactgttcactgtgagctttagagaaccagaacgatattaccttagacttttgcttctgcatgtaaaaggtgcgataagttttgaggatctgcgaactgtaggaggtgtaacttatgatacatttcatgaagctgctaaacaccgaggattattacttgatgacactatctggaaagatacgattgacgatgcaatcatccttaatatgcccaaacaactacggcaactttttgcatatatatgtgtgtttggatgtccttctgctgcagacaaattatgggatgagaataaatctcattttattgaagatttctgttggaaattacaccgaagagaaggtgcctgtgtgaactgtgaaatgcatgcccttaacgaaattcaggaggtattcacattgcatggaatgaaatgttcacatttcaaacttccggactatcctttattaatgaatgcaaatacatgtgatcaattgtacgagcaacaacaggcagaggttttgataaattctctgaatgatgaacagttggcagcctttcagactataacttcagccatcgaagatcaaactgtacaccccaaatgctttttcttggatggtccaggtggtagtggaaaaacatatctgtataaagttttaacacattatattagaggtcgtggtggtactgttttacccacagcatctacaggaattgctgcaaatttacttcttggtggaagaacctttcattcccaatataaattaccaattccattaaatgaaacttcaatttctagactcgatataaagagtgaagttgctaaaaccattaaaaaggcccaacttctcattattgatgaatgcaccatggcatccagtcatgctataaacgccatagatagattactaagagaaattatgaatttgaatgttgcatttggtgggaaagttctccttctcggaggggattttcgacaatgtctcagtattgtaccacatgctatgcgatcggccatagtacaaacgagtttaaagtactgtaatgtttggggatgtttcagaaagttgtctcttaaaacaaatatgagatcagaggattctgcttatagtgaatggttagtaaaacttggagatggcaaacttgatagcagttttcatttaggaatggatattattgaaatcccccatgaaatgatttgtaacggatctattattgaagctacctttggaaatagtatatctatagataatattaaaaatatatctaaacgtgcaattctttgtccaaaaaatgagcatgttcaaaaattaaatgaagaaattttggatatacttgatggagattttcacacatatttgagtgatgattccattgattcaacagatgatgctgaaaaggaaaattttcccatcgaatttcttaatagtattactccttcgggaatgccgtgtcataaattaaaattgaaagtgggtgcaatcatcatgctattgagaaatcttaatagtaaatggggtctttgtaatggtactagatttattatcaaaagattacgacctaacattatcgaagctgaagtattaacaggatctgcagagggagaggttgttctgattccaagaattgatttgtccccatctgacactggcctcccatttaaattaattcgaagacagtttcccgtgatgccagcatttgcgatgactattaataaatcacaaggacaaactctagacagagtaggaatattcctacctgaacccgttttcgcacatggtcagttatatgttgctttctctcgagttcgaagagcatgtgacgttaaagttaaagttgtaaatacttcatcacaagggaaattagtcaagcactctgaaagtgtttttactcttaatgtggtatacagggagatattagaataagtttaatcactttatcagtcattgtttgcatcaatgttgtttttatatcatgtttttgttgtttttatatcatgtctttgttgttgttatatcatgttgttattgtttatttattaataaatttatgtattattttcatatacattttactcatttcctttcatctctcacacttctattatagagaaagggcaaatagcaatattaaaatatttcctctaattaattccctttcaatgtgcacgaatttcgtgcaccgggccactag

SEQ ID NO: 104 - protein, 1496 aa, Artificial Sequence
  Helitron transposase
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 105 - DNA, 4682 bp, Artificial Sequence
  Tol2 transposase
cagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttaaatgttatttattctgcctatgaaaatcgttttcacattatatgaaattggtcagacatgttcattggtcctttggaagtgacgtcatgtcacatctattaccacaatgcacagcaccttgacctggaaattagggaaattataacagtcaatcagtggaagaaaatggaggaagtatgtgattcatcagcagctgcgagcagcacagtccaaaatcagccacaggatcaagagcacccgtggccgtatcttcgcgaattcttttctttaagtggtgtaaataaagattcattcaagatgaaatgtgtcctctgtctcccgcttaataaagaaatatcggccttcaaaagttcgccatcaaacctaaggaagcatattgaggtaagtacattaagtattttgttttactgatagtttttttttttttttttttttttttttttgggtgtgcatgttttgacgttgatggcgcgccttttatatgtgtagtaggcctattttcactaatgcatgcgattgacaatataaggctcacgtaataaaatgctaaaatgcatttgtaattggtaacgttaggtccacgggaaatttggcgcctattgcagctttgaataatcattatcattccgtgctctcattgtgtttgaattcatgcaaaacacaagaaaaccaagcgagaaatttttttccaaacatgttgtattgtcaaaacggtaacactttacaatgaggttgattagttcatgtattaactaacattaaataaccatgagcaatacatttgttactgtatctgttaatctttgttaacgttagttaatagaaatacagatgttcattgtttgttcatgttagttcacagtgcattaactaatgttaacaagatataaagtattagtaaatgttgaaattaacatgtatacgtgcagttcattattagttcatgttaactaatgtagttaactaacgaaccttattgtaaaagtgttaccatcaaaactaatgtaatgaaatcaattcaccctgtcatgtcagccttacagtcctgtgtttttgtcaatataatcagaaataaaattaatgtttgattgtcactaaatgctactgtatttctaaaatcaacaagtatttaacattataaagtgtgcaattggctgcaaatgtcagttttattaaagggttagttcacccaaaaatgaaaataatgtcattaatgactcgccctcatgtcgttccaagcccgtaagacctccgttcatcttcagaacacagtttaagatattttagatttagtccgagagctttctgtgcctccattgagaatgtatgtacggtatactgtccatgtccagaaaggtaataaaaacatcaaagtagtccatgtgacatcagtgggttagttagaattttttgaagcatcgaatacattttggtccaaaaataacaaaacctacgactttattcggcattgtattctcttccgggtctgttgtcaatccgcgttcacgacttcgcagtgacgctacaatgctgaataaagtcgtaggttttgttatttttggaccaaaatgtattttcgatgcttcaaataattctacctaacccactgatgtcacatggactactttgatgtttttattacctttctggacatggacagtataccgtacatacattttcagtggagggacagaaagctctcggactaaatctaaaatatcttaaactgtgttccgaagatgaacggaggtgttacgggcttggaacgacatgagggtgagtcattaatgacatcttttcatttttgggtgaactaaccctttaatgctgtaatcagagagtgtatgtgtaattgttacatttattgcatacaatataaatatttatttgttgtttttacagagaatgcacccaaattacctcaaaaactactctaaattgacagcacagaagagaaagatcgggacctccacccatgcttccagcagtaagcaactgaaagttgactcagttttcccagtcaaacatgtgtctccagtcactgtgaacaaagctatattaaggtacatcattcaaggacttcatcctttcagcactgttgatctgccatcatttaaagagctgattagtacactgcagcctggcatttctgtcattacaaggcctactttacgctccaagatagctgaagctgctctgatcatgaaacagaaagtgactgctgccatgagtgaagttgaatggattgcaaccacaacggattgttggactgcacgtagaaagtcattcattggtgtaactgctcactggatcaaccctggaagtcttgaaagacattccgctgcacttgcctgcaaaagattaatgggctctcatacttttgaggtactggccagtgccatgaatgatatccactcagagtatgaaatacgtgacaaggttgtttgcacaaccacagacagtggttccaactttatgaaggctttcagagtttttggtgtggaaaacaatgatatcgagactgaggcaagaaggtgtgaaagtgatgacactgattctgaaggctgtggtgagggaagtgatggtgtggaattccaagatgcctcacgagtcctggaccaagacgatggcttcgaattccagctaccaaaacatcaaaagtgtgcctgtcacttacttaacctagtctcaagcgttgatgcccaaaaagctctctcaaatgaacactacaagaaactctacagatctgtctttggcaaatgccaagctttatggaataaaagcagccgatcggctctagcagctgaagctgttgaatcagaaagccggcttcagcttttaaggccaaaccaaacgcggtggaattcaacttttatggctgttgacagaattcttcaaatttgcaaagaagcaggagaaggcgcacttcggaatatatgcacctctcttgaggttccaatgtaagtgtttttcccctctatcgatgtaaacaaatgtgggttgtttttgtttaatactctttgattatgctgatttctcctgtaggtttaatccagcagaaatgctgttcttgacagagtgggccaacacaatgcgtccagttgcaaaagtactcgacatcttgcaagcggaaacgaatacacagctggggtggctgctgcctagtgtccatcagttaagcttgaaacttcagcgactccaccattctctcaggtactgtgacccacttgtggatgccctacaacaaggaatccaaacacgattcaagcatatgtttgaagatcctgagatcatagcagctgccatccttctccctaaatttcggacctcttggacaaatgatgaaaccatcataaaacgaggtaaatgaatgcaagcaacatacacttgacgaattctaatctgggcaacctttgagccataccaaaattattcttttatttatttatttttgcactttttaggaatgttatatcccatctttggctgtgatctcaatatgaatattgatgtaaagtattcttgcagcaggttgtagttatccctcagtgtttcttgaaaccaaactcatatgtatcatatgtggtttggaaatgcagttagattttatgctaaaataagggatttgcatgattttagatgtagatgactgcacgtaaatgtagttaatgacaaaatccataaaatttgttcccagtcagaagcccctcaaccaaacttttctttgtgtctgctcactgtgcttgtaggcatggactacatcagagtgcatctggagcctttggaccacaagaaggaattggccaacagttcatctgatgatgaagattttttcgcttctttgaaaccgacaacacatgaagccagcaaagagttggatggatatctggcctgtgtttcagacaccagggagtctctgctcacgtttcctgctatttgcagcctctctatcaagactaatacacctcttcccgcatcggctgcctgtgagaggcttttcagcactgcaggattgcttttcagccccaaaagagctaggcttgacactaacaattttgagaatcagcttctactgaagttaaatctgaggttttacaactttgagtagcgtgtactggcattagattgtctgtcttatagtttgataattaaatacaaacagttctaaagcaggataaaaccttgtatgcatttcatttaatgttttttgagattaaaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 106 - protein, 649 aa, Artificial Sequence
  Tol2 transposase
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 107 - protein, 636 aa, Artificial Sequence
  TcBuster transposase
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 108 - DNA, 1911 bp, Artificial Sequence
  TcBuster transposase
atgatgttgaattggctgaaaagtggaaagcttgaaagtcaatcacaggaacagagttcctgctaccttgagaactctaactgcctgccaccaacgctcgattctacagatattatcggtgaagagaacaaagctggtaccacctctcgcaagaagcggaaatatgacgaggactatctgaacttcggttttacatggactggcgacaaggatgagcccaacggactttgtgtgatttgcgagcaggtagtcaacaattcctcacttaacccggccaaactgaaacgccatttggacacaaagcatccgacgcttaaaggcaagagcgaatacttcaaaagaaaatgtaacgagctcaatcaaaagaagcatacttttgagcgatacgtaagggacgataacaagaacctcctgaaagcttcttatctcgtcagtttgagaatagctaaacagggcgaggcatataccatagcggagaagttgatcaagccttgcaccaaggatctgacaacttgcgtatttggagaaaaattcgcgagcaaagttgatctcgtccccctgtccgacacgactatttcaaggcgaatcgaagacatgagttacttctgtgaagccgtgctggtgaacaggttgaaaaatgctaaatgtgggtttacgctgcagatggacgagtcaacagatgttgccggtcttgcaatcctgcttgtgtttgttaggtacatacatgaaagctcttttgaggaggatatgttgttctgcaaagcacttcccactcagacgacaggggaggagattttcaatcttctcaatgcctatttcgaaaagcactccatcccatggaatctgtgttaccacatttgcacagacggtgccaaggcaatggtaggagttattaaaggagtcatagcgagaataaaaaaactcgtccctgatataaaagctagccactgttgcctgcatcgccacgctttggctgtaaagcgaataccgaatgcattgcacgaggtgctcaatgacgctgttaaaatgatcaacttcatcaagtctcggccgttgaatgcgcgcgtcttcgctttgctgtgtgacgatttggggagcctgcataaaaatcttcttcttcataccgaagtgaggtggctgtctagaggaaaggtgctgacccgattttgggaactgagagatgaaattagaattttcttcaacgaaagggaatttgccgggaaattgaacgacaccagttggttgcaaaatttggcatatatagctgacatattcagttatctgaatgaagttaatctttccctgcaagggccgaatagcacaatcttcaaggtaaatagccgcattaacagtattaaatcaaagttgaagttgtgggaagagtgtataacgaaaaataacactgagtgttttgcgaacctcaacgattttttggaaacttcaaacactgcgttggatccaaacctgaagtctaatattttggaacatctcaacggtcttaagaacacctttctggagtattttccacctacgtgtaataatatctcctgggtggagaatcctttcaatgaatgcggtaacgtcgatacactcccaataaaagagagggaacaattgattgacatacggactgatacgacattgaaatcttcattcgtgcctgatggtataggaccattctggatcaaactgatggacgaatttccagaaattagcaaacgagctgtcaaagagctcatgccatttgtaaccacttacctctgtgagaaatcattttccgtctatgtagccacaaaaacaaaatatcgaaatagacttgatgctgaagacgatatgcgactccaacttactactatccatccagacattgacaacctttgtaacaacaagcaggctcagaaatcccactga

SEQ ID NO: 109 - protein, 107 aa, Artificial Sequence
  FKBP12 polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 110 - DNA, 321 bp, Artificial Sequence
  FKBP12 polynucleotide
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggag

SEQ ID NO: 111 - protein, 5 aa, Artificial Sequence
  linker
GGGGS

SEQ ID NO: 112 - DNA, 15 bp, Artificial Sequence
  linker
ggaggaggaggatcc

SEQ ID NO: 113 - protein, 282 aa, Artificial Sequence
  truncated caspase 9 polypeptide
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 114 - DNA, 843 bp, Artificial Sequence
  truncated caspase 9 polynucleotide
tttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

SEQ ID NO: 115 - protein, 394 aa, Artificial Sequence
  inducible proapoptotic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 116 - DNA, 1182 bp, Artificial Sequence
  inducible proapoptotic polypeptide
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccggatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

(19) United States
(12) Patent Application Publication
(10) Pub. No.: US 2022/0372105 A1
(43) Pub. Date: Nov. 24, 2022

(54) ALLOGENEIC CELL COMPOSITIONS AND METHODS OF USE

(71) Applicant: Poseida Therapeutics, Inc., San Diego, CA (US)

(72) Inventors: Eric M. OSTERTAG, San Diego, CA (US); Devon SHEDLOCK, San Diego, CA (US)

(21) Appl. No.: 17/640,176

(22) PCT Filed: Sep. 3, 2020
(86) PCT No.: PCT/US2020/049262

§ 371 Date: Mar. 3, 2022

Related U.S. Application Data
(60) Provisional application No. 62/976,536, filed on Feb. 14, 2020.
(60) Provisional application No. 62/896,495, filed on Sep. 5, 2019.

(51) Int. Cl. C07K 14/705; C12N 15/62; C07K 14/715; C07K 14/725

(52) U.S. Cl. CPC C07K 14/70507; C12N 15/625; C07K 14/70517; C07K 14/70521; C07K 14/70578; C07K 14/7155; C07K 14/7051; C07K 2319/02; C07K 2319/03

(57) ABSTRACT

Disclosed are chimeric stimulatory receptors (CSRs), cell compositions comprising CSRs, methods of making and methods of using same for the treatment of a disease or disorder in a subject.

### CROSS-REFERENCE TO RELATED APPLICATIONS

This application claims priority to, and the benefit of, U.S. Provisional Application No. 62/896,495, filed Sep. 5, 2019 and U.S. Provisional Application No. 62/976,536, filed Feb. 14, 2020. The contents of each of these applications are incorporated herein by reference in their entireties.

### FIELD OF THE DISCLOSURE

The disclosure is directed to molecular biology, and more, specifically, to chimeric receptors, allogeneic cell compositions, methods of making and methods of using the same.

### INCORPORATION-BY-REFERENCE OF SEQUENCE LISTING

The contents of the file named “POTH-055_001WO_SequenceListing_ST25.txt”, which was created on Aug. 21, 2020, and is 291 KB in size are hereby incorporated by reference in their entirety.

### BACKGROUND OF THE INVENTION

There has been a long-felt but unmet need in the art for an allogeneic cell composition that overcomes the challenges presented by eliminating genes involved in a graft versus host response and host versus graft response. The disclosure provides allogeneic cell compositions, methods of making and methods of using these compositions which comprise non-naturally occurring structural improvements to restore responsiveness of allogeneic cells to environmental stimuli as well as reduce or prevent rejection by natural killer cell-mediated cytotoxicity.

### SUMMARY OF THE INVENTION

The present disclosure provides a non-naturally occurring chimeric stimulatory receptor (CSR) comprising (a) an ectodomain comprising a signal peptide and an activation component, wherein the signal peptide comprises a CD2 signal peptide, and wherein the activation component comprises a CD2 extracellular domain or a portion thereof to which an agonist binds; (b) a transmembrane domain, wherein the transmembrane domain comprises a CD2 transmembrane domain or a portion thereof, and (c) an endodomain comprising a cytoplasmic domain and a signal transduction domain, wherein the cytoplasmic domain is a CD28 intracellular domain, a 4-1BB intracellular domain, an IL17RA intracellular domain, an IL15RA intracellular domain, an IL21R intracellular domain, an ICOS intracellular domain, a CD27 intracellular domain, an OX40 intracellular domain or a GITR intracellular domain or any combination thereof, and wherein the signal transduction domain comprises a CD3ζ protein or a portion thereof; and wherein the signal peptide and the cytoplasmic domain are not derived from the same protein.

In some aspects, the CD2 signal peptide comprises an amino acid sequence of at least 80%, at least 90%, at least 95%, at least 99% or 100% identical to SEQ ID NO: 5. In a preferred aspect, the CD2 signal peptide comprises an amino acid sequence of SEQ ID NO: 5.

The present disclosure also provides a non-naturally occurring chimeric stimulatory receptor (CSR) comprising (a) an ectodomain comprising a signal peptide and an activation component, wherein the signal peptide comprises a CD8a signal peptide, and wherein the activation component comprises a CD2 extracellular domain or a portion thereof to which an agonist binds; (b) a transmembrane domain, wherein the transmembrane domain comprises a CD2 transmembrane domain or a portion thereof, and (c) an endodomain comprising a cytoplasmic domain and a signal transduction domain, wherein the cytoplasmic domain is a CD2 intracellular domain, a CD28 intracellular domain, a 4-1BB intracellular domain, an IL17RA intracellular domain, an IL15RA intracellular domain, an IL21R intracellular domain, an ICOS intracellular domain, a CD27 intracellular domain, an OX40 intracellular domain or a GITR intracellular domain or any combination thereof, and wherein the signal transduction domain comprises a CD3ζ protein or a portion thereof, and wherein the signal peptide and the cytoplasmic domain are not derived from the same protein.

In some aspects, the CD8a signal peptide comprises an amino acid sequence of at least 80%, at least 90%, at least 95%, at least 99% or 100% identical to SEQ ID NO: 7. In a preferred aspect, the CD8a signal peptide comprises an amino acid sequence of SEQ ID NO: 7.

The present disclosure also provides a non-naturally occurring chimeric stimulatory receptor (CSR) wherein the activation component comprises a modification. In some aspects, the modification comprises a mutation or a truncation of the amino acid sequence of the CD2 extracellular domain or a portion thereof to which an agonist binds in comparison to the wildtype sequence of the CD2 extracellular domain or portion thereof. In some aspects, the non-naturally occurring CSR comprising a mutation or truncation of a CD2 extracellular domain or a portion thereof to which an agonist binds does not bind CD58. In some aspects, the CD2 extracellular domain or portion thereof comprising a mutation or truncation comprises an amino acid sequence of at least 80%, at least 85%, at least 90%, at least 95%, at least 99% or 100% identical to SEQ ID NO: 3. In a preferred aspect, the CD2 extracellular domain or portion thereof comprising the mutation or truncation comprises an amino acid sequence of SEQ ID NO: 3.

In some aspects, the CD2 transmembrane domain comprises an amino acid sequence of at least 80%, at least 90%, at least 95%, at least 99% or 100% identical to SEQ ID NO: 9. In a preferred aspect, the CD2 transmembrane domain or a portion thereof comprises an amino acid sequence of SEQ ID NO: 9.

In some aspects, the CD2 intracellular domain comprises an amino acid sequence of at least 80%, at least 90%, at least 95%, at least 99% or 100% identical to SEQ ID NO: 13. In a preferred aspect, the CD2 intracellular domain comprises an amino acid sequence of SEQ ID NO: 13. In some aspects, the CD28 intracellular domain comprises an amino acid sequence of at least 80%, at least 90%, at least 95%, at least 99% or 100% identical to SEQ ID NO: 15. In a preferred aspect, the CD28 intracellular domain comprises an amino acid sequence of SEQ ID NO: 15. In some aspects, the 4-1BB intracellular domain comprises an amino acid sequence of at least 80%, at least 90%, at least 95%, at least 99% or 100% identical to SEQ ID NO: 17. In a preferred aspect, the 4-1BB intracellular domain comprises an amino acid sequence of SEQ ID NO: 17. In some aspects, the IL17RA intracellular domain comprises an amino acid sequence of at least 80%, at least 90%, at least 95%, at least 99% or 100% identical to SEQ ID NO: 19. In a preferred aspect, the IL17RA intracellular domain comprises an amino acid sequence of SEQ ID NO: 19. In some aspects, the IL15RA intracellular domain comprises an amino acid sequence of at least 80%, at least 90%, at least 95%, at least 99% or 100% identical to SEQ ID NO: 21. In a preferred aspect, the IL15RA intracellular domain comprises an amino acid sequence of SEQ ID NO: 21. In some aspects, the IL21R intracellular domain comprises an amino acid sequence of at least 80%, at least 90%, at least 95%, at least 99% or 100% identical to SEQ ID NO: XX. In a preferred aspect, the IL21R intracellular domain comprises an amino acid sequence of SEQ ID NO: 23. In some aspects, the ICOS intracellular domain comprises an amino acid sequence of at least 80%, at least 90%, at least 95%, at least 99% or 100% identical to SEQ ID NO: the 25. In a preferred aspect, the ICOS intracellular domain comprises an amino acid sequence of SEQ ID NO: 25. In some aspects, the CD27 intracellular domain comprises an amino acid sequence of at least 80%, at least 90%, at least 95%, at least 99% or 100% identical to SEQ ID NO: the 27. In a preferred aspect, the CD27 intracellular domain comprises an amino acid sequence of SEQ ID NO: 27. In some aspects, the OX40 intracellular domain comprises an amino acid sequence of at least 80%, at least 90%, at least 95%, at least 99% or 100% identical to SEQ ID NO: the 29. In a preferred aspect, the OX40 intracellular domain comprises an amino acid sequence of SEQ ID NO: 29. In some aspects, the GITR intracellular domain comprises an amino acid sequence of at least 80%, at least 90%, at least 95%, at least 99% or 100% identical to SEQ ID NO: the 31. In a preferred aspect, the GITR intracellular domain comprises an amino acid sequence of SEQ ID NO: 31.

In some aspects, the signal transduction domain comprising a CD3ζ protein or a portion thereof comprises an amino acid sequence of at least 80%, at least 90%, at least 95%, at least 99% or 100% identical to SEQ ID NO: 11. In a preferred aspect, the signal transduction domain comprising a CD3ζ protein or a portion thereof comprises an amino acid sequence of SEQ ID NO: 11.

In some aspects, the non-naturally occurring CSR comprises an amino acid sequence at least 80%, at least 90%, at least 95%, at least 99% or 100% identical to SEQ ID NO: 39. In a preferred aspect, the non-naturally occurring CSR comprises an amino acid sequence of SEQ ID NO: 39. In some aspects, non-naturally occurring CSR comprises an amino acid sequence at least 80%, at least 90%, at least 95%, at least 99% or 100% identical to SEQ ID NO: 43. In a preferred aspect, the non-naturally occurring CSR comprises an amino acid sequence of SEQ ID NO: 43. In some aspects, the non-naturally occurring CSR comprises an amino acid sequence at least 80%, at least 90%, at least 95%, at least 99% or 100% identical to SEQ ID NO: 47. In a preferred aspect, the non-naturally occurring CSR comprises an amino acid sequence of SEQ ID NO: 47. In some aspects, the non-naturally occurring CSR comprises an amino acid sequence at least 80%, at least 90%, at least 95%, at least 99% or 100% identical to SEQ ID NO: 51. In a preferred aspect, the non-naturally occurring CSR comprises an amino acid sequence of SEQ ID NO: 51. In some aspects, the non-naturally occurring CSR comprises an amino acid sequence at least 80%, at least 90%, at least 95%, at least 99% or 100% identical to SEQ ID NO: 55. In a preferred aspect, the non-naturally occurring CSR comprises an amino acid sequence of SEQ ID NO: 55. In some aspects, the non-naturally occurring CSR comprises an amino acid sequence at least 80%, at least 90%, at least 95%, at least 99% or 100% identical to SEQ ID NO: 59. In a preferred aspect, the non-naturally occurring CSR comprises an amino acid sequence of SEQ ID NO: 59. In some aspects, the non-naturally occurring CSR comprises an amino acid sequence at least 80%, at least 90%, at least 95%, at least 99% or 100% identical to SEQ ID NO: 63. In a preferred aspect, the non-naturally occurring CSR comprises an amino acid sequence of SEQ ID NO: 63. In some aspects, the non-naturally occurring CSR comprises an amino acid sequence at least 80%, at least 90%, at least 95%, at least 99% or 100% identical to SEQ ID NO: 67. In a preferred aspect, the non-naturally occurring CSR comprises an amino acid sequence of SEQ ID NO: 67. In some aspects, the non-naturally occurring CSR comprises an amino acid sequence at least 80%, at least 90%, at least 95%, at least 99% or 100% identical to SEQ ID NO: 71. In a preferred aspect, the non-naturally occurring CSR comprises an amino acid sequence of SEQ ID NO: 71.

In some aspects, the non-naturally occurring CSR comprises an amino acid sequence at least 80%, at least 90%, at least 95%, at least 99% or 100% identical to SEQ ID NO: 37. In a preferred aspect, the non-naturally occurring CSR comprises an amino acid sequence of SEQ ID NO: 37. In some aspects, the non-naturally occurring CSR comprises an amino acid sequence at least 80%, at least 90%, at least 95%, at least 99% or 100% identical to SEQ ID NO: 41. In a preferred aspect, the non-naturally occurring CSR comprises an amino acid sequence of SEQ ID NO: 41. In some aspects, the non-naturally occurring CSR comprises an amino acid sequence at least 80%, at least 90%, at least 95%, at least 99% or 100% identical to SEQ ID NO: 45. In a preferred aspect, the non-naturally occurring CSR comprises an amino acid sequence of SEQ ID NO: 45. In some aspects, the non-naturally occurring CSR comprises an amino acid sequence at least 80%, at least 90%, at least 95%, at least 99% or 100% identical to SEQ ID NO: 49. In a preferred aspect, the non-naturally occurring CSR comprises an amino acid sequence of SEQ ID NO: 49. In some aspects, the non-naturally occurring CSR comprises an amino acid sequence at least 80%, at least 90%, at least 95%, at least 99% or 100% identical to SEQ ID NO: 53. In a preferred aspect, the non-naturally occurring CSR comprises an amino acid sequence of SEQ ID NO: 53. In some aspects, the non-naturally occurring CSR comprises an amino acid sequence at least 80%, at least 90%, at least 95%, at least 99% or 100% identical to SEQ ID NO: 57. In a preferred aspect, the non-naturally occurring CSR comprises an amino acid sequence of SEQ ID NO: 57. In some aspects, the non-naturally occurring CSR comprises an amino acid sequence at least 80%, at least 90%, at least 95%, at least 99% or 100% identical to SEQ ID NO: 61. In a preferred aspect, the non-naturally occurring CSR comprises an amino acid sequence of SEQ ID NO: 61. In some aspects, the non-naturally occurring CSR comprises an amino acid sequence at least 80%, at least 90%, at least 95%, at least 99% or 100% identical to SEQ ID NO: 65. In a preferred aspect, the non-naturally occurring CSR comprises an amino acid sequence of SEQ ID NO: 65. In some aspects, the non-naturally occurring CSR comprises an amino acid sequence at least 80%, at least 90%, at least 95%, at least 99% or 100% identical to SEQ ID NO: 69. In a preferred aspect, the non-naturally occurring CSR comprises an amino acid sequence of SEQ ID NO: 69. In some aspects, the non-naturally occurring CSR comprises an amino acid sequence at least 80%, at least 90%, at least 95%, at least 99% or 100% identical to SEQ ID NO: 73. In a preferred aspect, the non-naturally occurring CSR comprises an amino acid sequence of SEQ ID NO: 73.

The present disclosure provides a nucleic acid sequence encoding any CSR disclosed herein. The present disclosure provides a vector comprising a nucleic acid sequence encoding any CSR disclosed herein. The present disclosure provides a transposon comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

The present disclosure also provides a modified T lymphocyte (T-cell), comprising: (a) a modification of an endogenous sequence encoding a T-cell Receptor (TCR), wherein the modification reduces or eliminates a level of expression or activity of the TCR; and (b) any chimeric stimulatory receptor (CSR) disclosed herein. A modified T-cell disclosed herein can be an allogeneic cell or an autologous cell. In some preferred aspects, the modified cell is an allogeneic cell. In some preferred aspects, the modified cell is an allogeneic T-cell or a modified allogeneic CAR T-cell.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

The present disclosure provides a modified T lymphocyte (T-cell), comprising: (a) a modification of an endogenous sequence encoding a T-cell Receptor (TCR), wherein the modification reduces or eliminates a level of expression or activity of the TCR; and (b) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The modified T-cell can further comprise an inducible proapoptotic polypeptide. The modified T-cell can further comprise a modification of an endogenous sequence encoding Beta-2-Microglobulin (B2M), wherein the modification reduces or eliminates a level of expression or activity of a major histocompatibility complex (MHC) class I (MHC-I).

The modified T-cell can further comprise a non-naturally occurring polypeptide comprising an HLA class I histocompatibility antigen, alpha chain E (HLA-E) polypeptide. The non-naturally occurring polypeptide comprising a HLA-E polypeptide can further comprise a B2M signal peptide. The non-naturally occurring polypeptide comprising a HLA-E polypeptide can further comprise a B2M polypeptide. The non-naturally occurring polypeptide comprising an HLA-E polypeptide can further comprise a linker, wherein the linker is positioned between the B2M polypeptide and the HLA-E polypeptide. The non-naturally occurring polypeptide comprising an HLA-E polypeptide can further comprise a peptide and a B2M polypeptide. The non-naturally occurring polypeptide comprising an HLA-E can further comprise a first linker positioned between the B2M signal peptide and the peptide, and a second linker positioned between the B2M polypeptide and the peptide encoding the HLA-E.

The modified T-cell can further comprise a non-naturally occurring antigen receptor, a sequence encoding a therapeutic polypeptide, or a combination thereof. The non-naturally occurring antigen receptor can comprise a chimeric antigen receptor (CAR).

The CSR can be transiently expressed in the modified T-cell. The CSR can be stably expressed in the modified T-cell. The polypeptide comprising the HLA-E polypeptide can be transiently expressed in the modified T-cell. The polypeptide comprising the HLA-E polypeptide can be stably expressed in the modified T-cell. The inducible proapoptotic polypeptide can be transiently expressed in the modified T-cell. The inducible proapoptotic polypeptide can be stably expressed in the modified T-cell. The non-naturally occurring antigen receptor or a sequence encoding a therapeutic protein can be transiently expressed in the modified T-cell. The non-naturally occurring antigen receptor or a sequence encoding a therapeutic protein can be stably expressed in the modified T-cell.

The modified T-cell can be an autologous cell. The modified T-cell can be an allogeneic cell. The modified T-cell can be an early memory T cell, a stem cell-like T cell, a stem memory T cell (T_{SCM}), a central memory T cell (T_{CM}) or a stem cell-like T cell.

The present disclosure provides a composition comprising any modified T-cell disclosed herein. The present disclosure also provides a composition comprising a population of modified T lymphocytes (T-cells), wherein a plurality of the modified T-cells of the population comprise the CSR disclosed herein. The present disclosure also provides a composition comprising a population of T lymphocytes (T-cells), wherein a plurality of the T-cells of the population comprise the modified T-cell disclosed herein.

The present disclosure provides methods of treating a disease or disorder comprising administering to a subject in need thereof a therapeutically-effective amount of any composition disclosed herein; or a composition for use in the treatment of a disease or disorder. In one aspect, the composition is a modified T-cell or population of modified T-cells as disclosed herein. The present disclosure also a method of treating a disease or disorder comprising administering to a subject in need thereof a therapeutically-effective amount of a composition disclosed herein and at least one non-naturally occurring molecule that binds the CSR.

The present disclosure provides a method of producing a population of modified T-cells comprising, consisting essential of, or consisting of introducing into a plurality of primary human T-cells a composition comprising the CSR of the present disclosure or a sequence encoding the same to produce a plurality of modified T-cells under conditions that stably express the CSR within the plurality of modified T-cells and preserve desirable stem-like properties of the plurality of modified T-cells. The present disclosure provides a composition comprising a population of modified T-cells produced by the method. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprising the CSR expresses one or more cell-surface marker(s) of a stem memory T cell (T_{SCM}) or a T_{SCM}-like cell; and wherein the one or more cell-surface marker(s) comprise CD45RA and CD62L. some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses one or more cell-surface marker(s) of a central memory T cell (T_{CM}) or a T_{CM}-like cell; and wherein the one or more cell-surface marker(s) comprise CD45RO and CD62L. The composition can be for use in the treatment of a disease or disorder. The present disclosure also provides for use of a composition produced by the method for the treatment of a disease or disorder. The present disclosure further provides a method of treating a disease or disorder comprising administering to a subject in need thereof a therapeutically-effective amount of the composition produced by the method. The method of treating can further comprising administering an activator composition to the subject to activate the population of modified T-cells in vivo, to induce cell division of the population of modified T-cells in vivo, or a combination thereof.

The present disclosure provides a method of producing a population of modified T-cells comprising, consisting essential of, or consisting of introducing into a plurality of primary human T-cells a composition comprising the CSR of the present disclosure or a sequence encoding the same to produce a plurality of modified T-cells under conditions that transiently express the CSR within the plurality of modified T-cells and preserve desirable stem-like properties of the plurality of modified T-cells. The present disclosure provides a composition comprising a population of modified T-cells produced by the method. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprising the CSR expresses one or more cell-surface marker(s) of a stem memory T cell (T_{SCM}) or a T_{SCM}-like cell; and wherein the one or more cell-surface marker(s) comprise CD45RA and CD62L. some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses one or more cell-surface marker(s) of a central memory T cell (T_{CM}) or a T_{CM}-like cell; and wherein the one or more cell-surface marker(s) comprise CD45RO and CD62L. The composition can be for use in the treatment of a disease or disorder. The present disclosure also provides for use of a composition produced by the method for the treatment of a disease or disorder. The present disclosure further provides a method of treating a disease or disorder comprising administering to a subject in need thereof a therapeutically-effective amount of the composition produced by the method. In some aspects, the modified T-cells within the population of modified T-cells administered to the subject no longer express the CSR.

The present disclosure provides a method of expanding a population of modified T-cells comprising introducing into a plurality of primary human T-cells a composition comprising the CSR of the present disclosure or a sequence encoding the same to produce a plurality of modified T-cells under conditions that stably express the CSR within the plurality of modified T-cells and preserve desirable stem-like properties of the plurality of modified T-cells and contacting the cells with an activator composition to produce a plurality of activated modified T-cells, wherein expansion of the plurality of modified T-cells is at least two fold higher than the expansion of a plurality of wild-type T-cells not stably expressing the CSR under the same conditions. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprising the CSR expresses one or more cell-surface marker(s) of a stem memory T cell (T_{SCM}) or a T_{SCM}-like cell; and wherein the one or more cell-surface marker(s) comprise CD45RA and CD62L. some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses one or more cell-surface marker(s) of a central memory T cell (T_{CM}) or a T_{CM}-like cell; and wherein the one or more cell-surface marker(s) comprise CD45RO and CD62L. The present disclosure provides a composition comprising a population of modified T-cells expanded by the method. The composition can be for use in the treatment of a disease or disorder. The present disclosure also provides for use of a composition expanded by the method for the treatment of a disease or disorder. The present disclosure further provides a method of treating a disease or disorder comprising administering to a subject in need thereof a therapeutically-effective amount of the composition expanded by the method. The method of treating can further comprising administering an activator composition to the subject to activate the population of modified T-cells in vivo, to induce cell division of the population of modified T-cells in vivo, or a combination thereof.

The present disclosure provides a method of expanding a population of modified T-cells comprising introducing into a plurality of primary human T-cells a composition comprising the CSR of the present disclosure or a sequence encoding the same to produce a plurality of modified T-cells under conditions that transiently express the CSR within the plurality of modified T-cells and preserve desirable stem-like properties of the plurality of modified T-cells and contacting the cells with an activator composition to produce a plurality of activated modified T-cells, wherein expansion of the plurality of modified T-cells is at least two fold higher than the expansion of a plurality of wild-type T-cells not transiently expressing the CSR under the same conditions. The present disclosure provides a composition comprising a population of modified T-cells expanded by the method. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprising the CSR expresses one or more cell-surface marker(s) of a stem memory T cell (T_{SCM}) or a T_{SCM}-like cell; and wherein the one or more cell-surface marker(s) comprise CD45RA and CD62L. some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses one or more cell-surface marker(s) of a central memory T cell (T_{CM}) or a T_{CM}-like cell; and wherein the one or more cell-surface marker(s) comprise CD45RO and CD62L. The composition can be for use in the treatment of a disease or disorder. The present disclosure also provides for use of a composition expanded by the method for the treatment of a disease or disorder. The present disclosure further provides a method of treating a disease or disorder comprising administering to a subject in need thereof a therapeutically-effective amount of the composition expanded by the method. In some aspects, the modified T-cells within the population of modified T-cells administered to the subject no longer express the CSR.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.100, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing exemplary CSR CD2z-D111H mutants for enhanced manufacture of allogeneic or autologous CAR-T. CSR CD2z-D111H mutants can be delivered to allogeneic or autologous CAR-T cells during manufacturing to enhance cell growth and expansion, quality, survival, phenotype, function, subset composition, gene-editing efficiency, etc. These mutant CSRs can be delivered either transiently as encoded in mRNA or stably as encoded in a transposon.

FIG. 2 is a schematic diagram showing exemplary CSR CD2z-D111H mutants with CD8a signal peptide for enhanced manufacture of allogeneic or autologous CAR-T. CSR CD2z-D111H mutants can be delivered to allogeneic or autologous CAR-T cells during manufacturing to enhance cell growth and expansion, quality, survival, phenotype, function, subset composition, gene-editing efficiency, etc. These mutant CSRs can be delivered either transiently as encoded in mRNA or stably as encoded in a transposon.

FIG. 3 is a graph showing that the delivery of a CSR enhances CAR-T cell expansion during production. Pan T cells isolated from normal donor blood were genetically modified using the piggyBac® DNA modification system in combination with the Cas-CLOVER™ gene-editing system. Cells were electroporated in a single reaction with a transposon encoding at least a CAR and a selection gene, an mRNA encoding a CSR, an mRNA encoding the super piggyBac® transposase enzyme, an mRNA encoding Cas-CLOVER™, and multiple guide RNA (gRNA) targeting TCRb and b2M in order to knockout the TCR and MHCI (double-knockout; DKO). The cells were subsequently stimulated with agonist mAbs anti-CD2, anti-CD3 and anti-CD28, and were later selected for genetic modification over the course of a 14-day culture period. At the end of the initial culture period all T cells expressed the CAR, indicating successful selection for genetically-modified cells. In the samples expressing a CSR, a greater degree of expansion of the DKO cells was observed.

FIG. 4 is a schematic diagram showing the experimental protocol for assessing in vivo tumor control by CAR-T cells produced using different CSRs. Allogeneic CAR-T were produced using different boosters as described in FIGS. 1 and 3 and Tables 1 and 2. A murine xenograft model of multiple myeloma was utilized to assess in vivo anti-tumor efficacy of allo CAR-T cells produced with the 10 different boosters. Specifically, the RPMI-8226 cell line was injected subcutaneously (SC) into female NSG mice at a dose of 1×10⁷ cells (Day −7) and later treated with allo CAR-T cells by intravenous (IV) injection at a ‘stress’ dose (5×10⁶) on Day 0 when tumors were established (75-125 mm³ [target mean ˜100 mm³] by caliper measurement). The ‘stress’ dose was used for greater resolution in detecting possible functional differences in efficacy among the CAR-T cells produced with the different booster molecules.

FIG. 5 is a graph showing the tumor volume over time post-allo CAR-T cell treatment. In vivo tumor control was assessed according to the protocol shown in FIG. 4 and by using a ‘stress’ dose of CAR-T cells produced using different CSRs. Tumor volume assessment by caliper measurement for all animals as displayed as group averages with error bars as SEM (Standard Error of the Mean).

FIG. 6 is a graph showing the total T cells in blood over time post allo CAR-T cell treatment. In vivo tumor control was assessed according to the protocol shown in FIG. 4 and by using a ‘stress’ dose of CAR-T cells produced using different CSRs. Total T cells in blood is measured by TruCount staining for human CD45+ cells per μl (hCD45+/μL) for all animals as displayed as group averages with error bars as SEM.

FIG. 7 is a graph showing the peak T-cells (T cells Cmax) in blood. In vivo tumor control was assessed according to the protocol shown in FIG. 4 and by using a ‘stress’ dose of CAR-T cells produced using different CSRs. Peak levels of T cells in blood as measured by TruCount staining for human CD45+ (hCD45+) cells for all animals are displayed as group averages with error bars as SEM.

FIG. 8 is a graph showing the area under the curve of T cells (hCD45+) in blood. In vivo tumor control was assessed according to the protocol shown in FIG. 4 and by using a ‘stress’ dose of CAR-T cells produced using different CSRs. T cell AUC in blood as calculated from TruCount staining for human CD45+ cells for all animals as displayed as group averages with error bars as SEM.

FIG. 9 is a series of graphs showing the phenotype of CD8+ T cells in blood. In vivo tumor control was assessed according to the protocol shown in FIG. 4 and by using a ‘stress’ dose of CAR-T cells produced using different CSRs. Phenotype of CD8+ T cells in blood as measured by FACS staining for all animals as displayed as group averages with error bars as SEM at Days 14 and 35 post-CAR-T treatment. Cells were stained for expression of surface CD45RA, CD45RO, and CD62L to define TSCM, TCM, TEM, and TEFF cells; TSCM (CD45RA+CD45RO−CD62L+; blue), TCM (CD45RA−CD45RO+CD62L+; red), TEM (CD45RA−CD45RO+CD62L−; green), TEFF (CD45RA+CD45RO−CD62L−; purple).

All documents cited herein, including any cross referenced or related patent or application is hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### DETAILED DESCRIPTION OF THE INVENTION

The disclosure provides allogeneic cell compositions, methods of making and methods of using these compositions, which comprise non-naturally occurring structural improvements to restore responsiveness of allogeneic cells to environmental stimuli as well as reduce or prevent rejection by natural killer cell-mediated cytotoxicity.

Chimeric Stimulatory Receptors (CSRs) and Recombinant HLA-E Polypeptides

Adoptive cell compositions that are “universally” safe for administration to any patient requires a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated to prevent T-cell mediated GvH that could cause death to the subject. Thus, in a preferred aspect, the disclosure provides a pure TCR-negative allogeneic T-cell composition (e.g., each cell of the composition expresses at a level so low as to either be undetectable or non-existent).

Expression and/or function of MHC class I (MHC-I, specifically, HLA-A, HLA-B, and HLA-C) is reduced or eliminated to prevent HvG and, consequently, to improve engraftment of cells in a subject. Improved engraftment results in longer persistence of the cells, and, therefore, a larger therapeutic window for the subject. Specifically, expression and/or function of a structural element of MHC-I, Beta-2-Microglobulin (B2M), is reduced or eliminated.

The above strategies induce further challenges. T Cell Receptor (TCR) knockout (KO) in T cells results in loss of expression of CD3-zeta (CD3z or CD3ζ), which is part of the TCR complex. The loss of CD3ζ in TCR-KO T-cells dramatically reduces the ability of optimally activating and expanding these cells using standard stimulation/activation reagents, including, but not limited to, agonist anti-CD3 mAb. When the expression or function of any one component of the TCR complex is interrupted, all components of the complex are lost, including TCR-alpha (TCRα), TCR-beta (TCRβ), CD3-gamma (CD3γ), CD3-epsilon (CD3ε), CD3-delta (CD3δ), and CD3-zeta (CD3ζ). Both CD3ε and CD3ζ are required for T cell activation and expansion. Agonist anti-CD3 mAbs typically recognize CD3ε and possibly another protein within the complex which, in turn, signals to CD3ζ. CD3ζ provides the primary stimulus for T cell activation (along with a secondary co-stimulatory signal) for optimal activation and expansion. Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb. In fact, T cell expansion is reduced to only 20-40% of the normal level of expansion when stimulated using standard activation/stimulation reagents, including agonist anti-CD3 mAb.

Thus, the present disclosure provides a non-naturally occurring chimeric stimulatory receptor (CSR) comprising: (a) an ectodomain comprising a signal peptide and an activation component; (b) a transmembrane domain; and (c) an endodomain comprising a cytoplasmic domain and a signal transduction domain; and wherein the signal peptide and the cytoplasmic domain are not derived from the same protein.

The activation component can comprise a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds. The activation component can comprise a CD2 extracellular domain or a portion thereof to which an agonist binds.

The signal transduction domain can comprise one or more of a component of a human signal transduction domain, T-cell Receptor (TCR), a component of a TCR complex, a component of a TCR co-receptor, a component of a TCR co-stimulatory protein, a component of a TCR inhibitory protein, a cytokine receptor, and a chemokine receptor. The signal transduction domain can comprise a CD3 protein or a portion thereof. The CD3 protein can comprise a CD3ζ protein or a portion thereof.

The activation domain can be isolated or derived from a first protein. The signal peptide can be isolated or derived from a second protein. The transmembrane domain can ve isolated or derived from a third protein. The cytoplasmic domain can be isolated or derived from a fourth protein. The signaling domain can be isolated or derived from a fifth protein. The first protein and the second protein can be identical. The first protein and the third protein can be identical. The first protein and the fourth protein can not be identical. The first protein and the fifth protein can not be identical. The second protein and the third protein can be identical. The second protein and the fourth protein can not be identical. The second protein and the fifth protein can not be identical. The third protein and the fourth protein can not be identical. The third protein and the fifth protein can not be identical. The fourth protein and the fifth protein can not be identical.

In some aspects, the activation component does not bind a naturally-occurring molecule. In some aspects, the activation component binds a naturally-occurring molecule but the CSR does not transduce a signal upon binding of the activation component to a naturally-occurring molecule. In some aspects, the activation component binds to a non-naturally occurring molecule. In some aspects, the activation component does not bind a naturally-occurring molecule but binds a non-naturally occurring molecule. The CSR can selectively transduces a signal upon binding of the activation component to a non-naturally occurring molecule.

The present disclosure provides a nucleic acid sequence encoding any CSR disclosed herein. The present disclosure provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

A modified cell disclosed herein can be an allogeneic cell or an autologous cell. In some preferred aspects, the modified cell is an allogeneic cell. In some aspects, the modified cell is an autologous T-cell or a modified autologous CAR T-cell. In some preferred aspects, the modified cell is an allogeneic T-cell or a modified allogeneic CAR T-cell.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

The present disclosure provides a modified T lymphocyte (T-cell), comprising: (a) a modification of an endogenous sequence encoding a T-cell Receptor (TCR), wherein the modification reduces or eliminates a level of expression or activity of the TCR; and (b) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The modified T-cell can further comprise an inducible proapoptotic polypeptide. The modified T-cell can further comprise a modification of an endogenous sequence encoding Beta-2-Microglobulin (B2M), wherein the modification reduces or eliminates a level of expression or activity of a major histocompatibility complex (MHC) class I (MHC-I).

The modified T-cell can further comprise a non-naturally occurring polypeptide comprising an HLA class I histocompatibility antigen, alpha chain E (HLA-E) polypeptide. The non-naturally occurring polypeptide comprising a HLA-E polypeptide can further comprise a B2M signal peptide. The non-naturally occurring polypeptide comprising a HLA-E polypeptide can further comprise a B2M polypeptide. The non-naturally occurring polypeptide comprising an HLA-E polypeptide can further comprise a linker, wherein the linker is positioned between the B2M polypeptide and the HLA-E polypeptide. The non-naturally occurring polypeptide comprising an HLA-E polypeptide can further comprise a peptide and a B2M polypeptide. The non-naturally occurring polypeptide comprising an HLA-E can further comprise a first linker positioned between the B2M signal peptide and the peptide, and a second linker positioned between the B2M polypeptide and the peptide encoding the HLA-E.

The modified T-cell can further comprise a non-naturally occurring antigen receptor, a sequence encoding a therapeutic polypeptide, or a combination thereof. The non-naturally occurring antigen receptor can comprise a chimeric antigen receptor (CAR).

The CSR can be transiently expressed in the modified T-cell. The CSR can be stably expressed in the modified T-cell. The polypeptide comprising the HLA-E polypeptide can be transiently expressed in the modified T-cell. The polypeptide comprising the HLA-E polypeptide can be stably expressed in the modified T-cell. The inducible proapoptotic polypeptide can be transiently expressed in the modified T-cell. The inducible proapoptotic polypeptide can be stably expressed in the modified T-cell. The non-naturally occurring antigen receptor or a sequence encoding a therapeutic protein can be transiently expressed in the modified T-cell. The non-naturally occurring antigen receptor or a sequence encoding a therapeutic protein can be stably expressed in the modified T-cell.

Gene editing compositions, including but not limited to, RNA-guided fusion proteins comprising dCas9-Clo051, as described in detail herein, can be used to target and decrease or eliminate expression of an endogenous T-cell receptor. In preferred aspects, the gene editing compositions target and delete a gene, a portion of a gene, or a regulatory element of a gene (such as a promoter) encoding an endogenous T-cell receptor. Non-limiting examples of primers (including a T7 promoter, genome target sequence, and gRNA scaffold) for the generation of guide RNA (gRNA) templates for targeting and deleting TCR-alpha (TCR-α), targeting and deleting TCR-beta (TCR-β), and targeting and deleting beta-2-microglobulin (β2M) are disclosed in PCT Application No. PCT/US2019/049816.

Gene editing compositions, including but not limited to, RNA-guided fusion proteins comprising dCas9-Clo051, can be used to target and decrease or eliminate expression of an endogenous MHCI, MHCII, or MHC activator. In preferred aspects, the gene editing compositions target and delete a gene, a portion of a gene, or a regulatory element of a gene (such as a promoter) encoding one or more components of an endogenous MHCI, MHCII, or MHC activator. Non-limiting examples of guide RNAs (gRNAs) for targeting and deleting MHC activators are disclosed in PCT Application No. PCT/US2019/049816 (incorporated herein by reference in its entirety).

A detailed description of non-naturally occurring chimeric stimulatory receptors, genetic modifications of endogenous sequences encoding TCR-alpha (TCR-α), TCR-beta (TCR-β), and/or Beta-2-Microglobulin (β2M), and non-naturally occurring polypeptides comprising an HLA class I histocompatibility antigen, alpha chain E (HLA-E) polypeptide is disclosed in PCT Application No. PCT/US2019/049816 (incorporated herein by reference in its entirety).

Chimeric Stimulatory Receptors of the Disclosure

The present disclosure provides a chimeric stimulatory receptor (CSR) comprising an activation component comprising, consisting essentially of or consisting of a CD2 extracellular domain or a portion thereof to which an agonist binds. The CD2 extracellular domain or portion thereof to which an agonist binds comprises, consists essentially of or consists of an amino acid sequence at least 95%, 96%, 97%, 98%, 99% or 100% (or any percentage in between) identical to: KEITNALETWGALGQDINLDIPSFQMSDDIDDIKWEKTSDKKKIAQFRKEKETFKEKDTYKL FKNGTLKIKHLKTDDQDIYKVSIYDTKGKNVLEKIFDLKIQERVSKPKISWTCINTTLTCEV MNGTDPELNLYQDGKHLKLSQRVITHKWTTSLSAKFKCTAGNKVSKESSVEPVSCPEKGLD (SEQ ID NO: 1). In a preferred aspect, the CD2 extracellular domain or portion thereof to which an agonist binds comprises, consists essentially of, or consists of the amino acid sequence of SEQ ID NO: 1.

In some aspects, the CD2 extracellular domain or portion thereof to which an agonist binds is encoded by a polynucleotide comprising, consisting essentially of or consisting of a nucleic acid sequence at least 95%, 96%, 97%, 98%, 99% or 100% (or any percentage in between) identical to: AAAGAGATCACAAACGCCCTGGAAACCTGGGGAGCCCTCGGCCAGGATATTAACCTGGACAT CCCCAGCTTCCAGATGAGCGACGACATCGATGACATCAAGTGGGAGAAAACCAGCGACAAGA AGAAGATCGCCCAGTTCCGGAAAGAGAAAGAGACATTCAAAGAGAAGGACACCTACAAGCTG TTCAAGAACGGCACCCTGAAGATCAAGCACCTGAAAACCGACGACCAGGACATCTATAAGGT GTCCATCTACGACACCAAGGGCAAGAACGTGCTGGAAAAGATCTTCGACCTCAAGATCCAAG AGCGGGTGTCCAAGCCTAAGATCAGCTGGACCTGCATCAACACCACACTGACCTGCGAAGTG ATGAACGGCACAGACCCCGAGCTGAACCTGTACCAGGATGGCAAACACCTGAAGCTGAGCCA GCGCGTGATCACCCACAAGTGGACAACAAGCCTGAGCGCCAAGTTCAAGTGCACCGCCGGAA ACAAAGTGTCTAAAGAGTCCAGCGTCGAGCCCGTGTCTTGCCCTGAAAAAGGACTGGAC (SEQ ID NO: 2). In a preferred aspect, the CD2 extracellular domain or portion thereof to which an agonist binds is encoded by a polynucleotide comprising, consisting essentially of or consisting of a nucleic acid sequence of SEQ ID NO: 2.

The present disclosure provides a chimeric stimulatory receptor (CSR) comprising an ectodomain comprising an activation component comprising, consisting essentially of or consisting of a non-naturally occurring CD2 extracellular domain. In some aspects, the ectodomain of the CSR of the present disclosure can comprise a modification. The modification can comprise a mutation or a truncation in the amino acid sequence of the activation component in comparison to a wild type amino acid sequence of the activation component. The mutation or a truncation in the amino acid sequence of the activation component can comprise a mutation or truncation of a CD2 extracellular domain or a portion thereof to which an agonist binds. The mutated or truncated CD2 extracellular domain binds anti-CD2 activating agonists and anti-CD2 activating molecules but does not bind naturally occurring CD58. In some aspects, the mutation occurring in the CD2 extracellular domain that binds anti-CD2 activating agonists but does not bind CD58 is a D111H mutation. The CD2 extracellular domain with a D111H mutation comprises, consists essentially of or consists of an amino acid sequence at least 95%, 96%, 97%, 98%, 99% or 100% (or any percentage in between) identical to: KEITNALETWGALGQDINLDIPFQMSDDIDDIKWEKTSDKKKIAQFRKEKETFKEKDTYKL FKNGTLKIKHLKTDDQDIYKVSIYHTKGKNVLEKIFDLKIQERVSKPKISWTCINTTLTCEV MNGTDPELNLYQDGKHLKLSQRVITHKWTTSLSAKFKCTAGNKVSKESSVEPVSCPEKGLD (SEQ ID NO: 3). In a preferred aspect, the CD2 extracellular domain with a D111H mutation comprises, consists essentially of, or consists of the amino acid sequence of SEQ ID NO: 3.

In some aspects, the CD2 extracellular domain with a D111H mutation is encoded by a polynucleotide comprising, consisting essentially of or consisting of a nucleic acid sequence at least 95%, 96%, 97%, 98%, 99% or 100% (or any percentage in between) identical to: AAAGAAATCACGAATGCATTGGAAACCTGGGGAGCCCTCGGCCAGGATATTAACCTGGACAT CCCCAGCTTCCAGATGAGCGACGACATCGATGACATCAAGTGGGAGAAAACCAGCGACAAGA AGAAGATCGCCCAGTTCCGGAAAGAGAAAGAGACATTCAAAGAGAAGGACACCTACAAGCTG TTCAAGAACGGCACCCTGAAGATCAAGCACCTGAAAACCGACGACCAGGACATCTATAAGGT GTCCATCTACCACACCAAGGGCAAGAACGTGCTGGAAAAGATCTTCGACCTCAAGATCCAAG AGCGGGTGTCCAAGCCTAAGATCAGCTGGACCTGCATCAACACCACACTGACCTGCGAAGTG ATGAACGGCACAGACCCCGAGCTGAACCTGTACCAGGATGGCAAACACCTGAAGCTGAGCCA GCGCGTGATCACCCACAAGTGGACAACAAGCCTGAGCGCCAAGTTCAAGTGCACCGCCGGAA ACAAAGTGTCTAAAGAGTCCAGCGTCGAGCCCGTGTCTTGCCCTGAAAAAGGACTGGAC (SEQ ID NO: 4). In a preferred aspect, the CD2 extracellular domain with a D111H mutation is encoded by a polynucleotide comprising, consisting essentially of or consisting of a nucleic acid sequence of SEQ ID NO: 4.

The endodomain of a CSR of the present disclosure can further comprise, consist essentially of, or consist of a signal peptide. In some aspects, the signal peptide can comprise, consist essentially of, or consist of a CD2 signal peptide or a portion thereof. In some aspects, the signal peptide can comprise, consist essentially of, or consist of a CD8a signal peptide or a portion thereof.

In some aspect, the CD2 signal peptide comprises, consists essentially of or consists of an amino acid sequence at least 95%, 96%, 97%, 98%, 99% or 100% (or any percentage in between) identical to: MSFPCKFVASFLLIFNVSSKGAVS (SEQ ID NO: 5). In a preferred aspect, the CD2 signal peptide comprises, consists essentially of, or consists of the amino acid sequence of SEQ ID NO: 5.

In some aspects, the CD2 signal peptide is encoded by a polynucleotide comprising, consisting essentially of or consisting of a nucleic acid sequence at least 95%, 96%, 97%, 98%, 99% or 100% (or any percentage in between) identical to: ATGAGCTTCCCTTGCAAGTTCGTGGCCAGCTTCCTGCTGATCTTCAACGTGTCCTCTAAGGG CGCCGTGTCC (SEQ ID NO: 6). In a preferred aspect, the CD2 signal peptide is encoded by a polynucleotide comprising, consisting essentially of or consisting of a nucleic acid sequence of SEQ ID NO: 6.

In some aspect, the CD8a signal peptide comprises, consists essentially of or consists of an amino acid sequence at least 95%, 96%, 97%, 98%, 99% or 100% (or any percentage in between) identical to: MALPVTALLLPLALLLHAARP (SEQ ID NO: 7). In a preferred aspect, the CD8a signal peptide comprises, consists essentially of, or consists of the amino acid sequence of SEQ ID NO: 7.

In some aspects, the CD8a signal peptide is encoded by a polynucleotide comprising, consisting essentially of or consisting of a nucleic acid sequence at least 95%, 96%, 97%, 98%, 99% or 100% (or any percentage in between) identical to: ATGGCTCTGCCTGTGACAGCTCTGCTGCTGCCTCTGGCTCTGCTTCTGCATGCCGCCAGACC T (SEQ ID NO: 8). In a preferred aspect, the CD8a signal peptide is encoded by a polynucleotide comprising, consisting essentially of or consisting of a nucleic acid sequence of SEQ ID NO: 8.

The present disclosure provides a CSR comprising a transmembrane domain. In some aspects, the transmembrane domain can comprise, consist essentially or consist of a CD2 transmembrane domain or a portion thereof. In some aspects, the CD2 transmembrane domain or a portion thereof comprises, consists essentially of or consists of an amino acid sequence at least 95%, 96%, 97%, 98%, 99% or 100% (or any percentage in between) identical to: IYLIIGICGGGSLLMVFVALLVFYIT (SEQ ID NO: 9). In a preferred aspect, the CD2 transmembrane domain or a portion thereof comprises, consists essentially of, or consists of the amino acid sequence of SEQ ID NO: 9.

In some aspects, the CD2 transmembrane domain or a portion thereof is encoded by a polynucleotide comprising, consisting essentially of or consisting of a nucleic acid sequence at least 95%, 96%, 97%, 98%, 99% or 100% (or any percentage in between) identical to: ATCTACCTGATCATCGGCATCTGTGGCGGCGGATCCCTGCTGATGGTGTTTGTGGCTCTGCT GGTGTTCTACATCACC (SEQ ID NO: 10). In a preferred aspect, the CD2 transmembrane domain or a portion thereof is encoded by a polynucleotide comprising, consisting essentially of or consisting of a nucleic acid sequence of SEQ ID NO: 10.

This disclosure provides a CSR comprising an endodomain comprising at least one signal transduction domain. In some aspects, the signal transduction domain can comprise, consist essentially or consist of a CD3ζ intracellular domain or a portion thereof. In some aspects, the CD3ζ intracellular domain or a portion thereof comprises, consists essentially of or consists of an amino acid sequence at least 95%, 96%, 97%, 98%, 99% or 100% (or any percentage in between) identical to: RVKFSRSADAPAYKQGQNQLYNELNLGRREEYDVLDKRRGRDPEMGGKPRRKNPQEGLYNEL QKDKMAEAYSEIGMKGERRRGKGHDGLYQGLSTATKDTYDALHMQALPPR (SEQ ID NO: 11). In a preferred aspect, the CD3ζ intracellular domain or a portion thereof comprises, consists essentially of, or consists of the amino acid sequence of SEQ ID NO: 11.

In some aspects, the CD3ζ intracellular domain or a portion thereof is encoded by a polynucleotide comprising, consisting essentially of or consisting of a nucleic acid sequence at least 95%, 96%, 97%, 98%, 99% or 100% (or any percentage in between) identical to: AGAGTGAAATTCAGCCGCAGCGCCGATGCTCCTGCCTATAAGCAGGGACAGAACCAGCTGTA CAACGAGCTGAATCTGGGGCGCAGAGAAGAGTACGATGTGCTGGACAAGCGGAGAGGCAGAG ATCCTGAGATGGGCGGCAAGCCCAGACGGAAGAATCCTCAAGAGGGCCTGTATAATGAGCTG CAGAAAGACAAGATGGCCGAGGCCTACAGCGAGATCGGAATGAAGGGCGAGCGCAGAAGAGG CAAGGGACACGATGGACTGTATCAGGGCCTGAGCACCGCCACCAAGGATACCTATGATGCCC TGCACATGCAGGCCCTGCCTCCAAGA (SEQ ID NO: 12). In a preferred aspect, the CD3ζ intracellular domain or a portion thereof is encoded by a polynucleotide comprising, consisting essentially of or consisting of a nucleic acid sequence of SEQ ID NO: 12.

The endodomain of a CSR of the present disclosure can further comprise, consist essentially of, or consist of a cytoplasmic domain. In some aspects, the cytoplasmic domain can comprise, consist essentially or consist of a CD2 intracellular domain (ICD) or a portion thereof. In some aspects, the cytoplasmic domain can comprise, consist essentially or consist of a CD28 intracellular domain or a portion thereof. In some aspects, the cytoplasmic domain can comprise, consist essentially or consist of a 4-1BB intracellular domain or a portion thereof. In some aspects, the cytoplasmic domain can comprise, consist essentially or consist of a IL17RA intracellular domain or a portion thereof. In some aspects, the cytoplasmic domain can comprise, consist essentially or consist of a IL15RA intracellular domain or a portion thereof. In some aspects, the cytoplasmic domain can comprise, consist essentially or consist of a IL21R intracellular domain or a portion thereof. In some aspects, the cytoplasmic domain can comprise, consist essentially or consist of a ICOS intracellular domain or a portion thereof. In some aspects, the cytoplasmic domain can comprise, consist essentially or consist of a CD27 intracellular domain or a portion thereof. In some aspects, the cytoplasmic domain can comprise, consist essentially or consist of a OX40 intracellular domain or a portion thereof. In some aspects, the cytoplasmic domain can comprise, consist essentially or consist of a GITR intracellular domain or a portion thereof.

In some aspects, the CD2 intracellular domain or a portion thereof comprises, consists essentially of or consists of an amino acid sequence at least 95%, 96%, 97%, 98%, 99% or 100% (or any percentage in between) identical to: KRKKQRSRRNDEELETRAHRVATEERGRKPHQIPASTPQNPATSQHPPPPPGHRSQAPSHRP PPPGHRVQHQPQKRPPAPSGTQVHQQKGPPLPRPRVQPKPPHGAAENSLSPSSN (SEQ ID NO: 13). In a preferred aspect, the CD2 intracellular domain or a portion thereof comprises, consists essentially of, or consists of the amino acid sequence of SEQ ID NO: 13.

In some aspects, the CD2 intracellular domain or a portion thereof is encoded by a polynucleotide comprising, consisting essentially of or consisting of a nucleic acid sequence at least 95%, 96%, 97%, 98%, 99% or 100% (or any percentage in between) identical to: AAGCGGAAGAAGCAGCGGAGCAGACGGAACGACGAGGAACTGGAAACACGGGCCCATAGAGT GGCCACCGAGGAAAGAGGCAGAAAGCCCCACCAGATTCCAGCCAGCACACCCCAGAATCCTG CCACCTCTCAACACCCTCCACCTCCACCTGGACACAGATCTCAGGCCCCATCTCACAGACCT CCACCACCTGGTCATCGGGTGCAGCACCAGCCTCAGAAAAGACCTCCTGCTCCTAGCGGCAC ACAGGTGCACCAGCAAAAAGGACCTCCACTGCCTCGGCCTAGAGTGCAGCCTAAACCTCCTC ATGGCGCCGCTGAGAACAGCCTGTCTCCAAGCAGCAAC (SEQ ID NO: 14). In a preferred aspect, the CD2 intracellular domain or a portion thereof is encoded by a polynucleotide comprising, consisting essentially of or consisting of a nucleic acid sequence of SEQ ID NO: 14.

In some aspects, the CD28 intracellular domain or a portion thereof comprises, consists essentially of or consists of an amino acid sequence at least 95%, 96%, 97%, 98%, 99% or 100% (or any percentage in between) identical to: RSKRSRLLHSDYMNMTPRRPGPTRKHYQPYAPPRDFAAYRS (SEQ ID NO: 15). In a preferred aspect, the CD28 intracellular domain or a portion thereof comprises, consists essentially of, or consists of the amino acid sequence of SEQ ID NO: 15.

In some aspects, the CD28 intracellular domain or a portion thereof is encoded by a polynucleotide comprising, consisting essentially of or consisting of a nucleic acid sequence at least 95%, 96%, 97%, 98%, 99% or 100% (or any percentage in between) identical to: AGAAGCAAGCGGAGCCGGCTGCTGCACAGCGACTACATGAACATGACCCCTAGACGGCCCGG ACCTACCAGAAAGCACTACCAGCCTTACGCTCCTCCTAGAGACTTCGCCGCCTACCGGTCC (SEQ ID NO: 16). In a preferred aspect, the CD28 intracellular domain or a portion thereof is encoded by a polynucleotide comprising, consisting essentially of or consisting of a nucleic acid sequence of SEQ ID NO: 16.

In some aspects, the 4-1BB intracellular domain or a portion thereof comprises, consists essentially of or consists of an amino acid sequence at least 95%, 96%, 97%, 98%, 99% or 100% (or any percentage in between) identical to: KRGRKKLLYIFKQPFMRPVQTTQEEDGCSCRFPEEEEGGCEL (SEQ ID NO: 17). In a preferred aspect, the 4-1BB intracellular domain or a portion thereof comprises, consists essentially of, or consists of the amino acid sequence of SEQ ID NO: 17.

In some aspects, the 4-1BB intracellular domain or a portion thereof is encoded by a polynucleotide comprising, consisting essentially of or consisting of a nucleic acid sequence at least 95%, 96%, 97%, 98%, 99% or 100% (or any percentage in between) identical to: AAGCGGGGCAGAAAGAAGCTGCTGTACATCTTCAAGCAGCCCTTCATGCGGCCCGTGCAGAC CACACAAGAGGAAGATGGCTGCTCCTGCAGATTCCCCGAGGAAGAAGAAGGCGGCTGCGAGC TG (SEQ ID NO: 18). In a preferred aspect, the 4-1BB intracellular domain or a portion thereof is encoded by a polynucleotide comprising, consisting essentially of or consisting of a nucleic acid sequence of SEQ ID NO: 18.

In some aspects, the IL17RA intracellular domain or a portion thereof comprises, consists essentially of or consists of an amino acid sequence at least 95%, 96%, 97%, 98%, 99% or 100% (or any percentage in between) identical to: KKRIKPIVWPSLPDHKKTLEHLCKKPRKNLNVSFNPESFLDCQIHRVDDIQARDEVEGFLQD TFPQQLEESEKQRLGGDVQSPNCPSEDVVITPESFGRDSSLTCLAGNVSACDAPILSSSRSL DCRESGKNGPHVYQDLLLSLGTTNSTLPPPFSLQSGILTLNPVAQGQPILTSLGSNQEEAYV TMSSFYQNQ (SEQ ID NO: 19). In a preferred aspect, the IL17RA intracellular domain or a portion thereof comprises, consists essentially of, or consists of the amino acid sequence of SEQ ID NO: 19.

In some aspects, the IL17RA intracellular domain or a portion thereof is encoded by a polynucleotide comprising, consisting essentially of or consisting of a nucleic acid sequence at least 95%, 96%, 97%, 98%, 99% or 100% (or any percentage in between) identical to: AAGAAGCGGATCAAGCCCATCGTGTGGCCCAGCCTGCCTGACCACAAGAAAACCCTGGAACA CCTGTGCAAGAAGCCCCGGAAGAACCTGAATGTGTCCTTCAATCCCGAGAGCTTCCTGGACT GCCAGATCCACAGAGTGGACGACATCCAGGCCAGAGATGAGGTGGAAGGCTTTCTGCAGGAC ACCTTTCCACAGCAGCTGGAAGAGAGCGAGAAGCAGAGACTCGGCGGAGATGTGCAGAGCCC TAATTGCCCTAGCGAGGACGTGGTCATCACCCCTGAGAGCTTCGGCAGAGATAGCAGCCTGA CATGTCTGGCCGGCAATGTGTCCGCCTGTGATGCCCCTATCCTGAGCAGCAGCAGAAGCCTG GATTGCAGAGAGAGCGGAAAGAACGGCCCTCATGTGTATCAGGACCTGCTGCTGAGCCTGGG CACCACCAATTCTACACTGCCTCCACCATTCAGCCTGCAGAGCGGCATCCTGACACTGAACC CTGTTGCTCAGGGCCAGCCAATCCTGACAAGCCTGGGCTCCAATCAAGAAGAGGCCTACGTC ACCATGTCCAGCTTCTACCAGAACCAG (SEQ ID NO 20). In a preferred aspect, the IL17RA intracellular domain or a portion thereof is encoded by a polynucleotide comprising, consisting essentially of or consisting of a nucleic acid sequence of SEQ ID NO: 20.

In some aspects, the IL15RA intracellular domain or a portion thereof comprises, consists essentially of or consists of an amino acid sequence at least 95%, 96%, 97%, 98%, 99% or 100% (or any percentage in between) identical to: KSRQTPPLASVEMEAMEALPVTWGTSSRDEDLENCSHHL (SEQ ID NO: 21). In a preferred aspect, the IL15RA intracellular domain or a portion thereof comprises, consists essentially of, or consists of the amino acid sequence of SEQ ID NO: 21.

In some aspects, the IL15RA intracellular domain or a portion thereof is encoded by a polynucleotide comprising, consisting essentially of or consisting of a nucleic acid sequence at least 95%, 96%, 97%, 98%, 99% or 100% (or any percentage in between) identical to: AAGAGCCGGCAGACACCTCCTCTGGCCAGCGTGGAAATGGAAGCCATGGAAGCTCTGCCTGT GACCTGGGGCACAAGCAGCAGAGATGAGGACCTGGAAAACTGCAGCCACCACCTG (SEQ ID NO: 22). In a preferred aspect, the IL15RA intracellular domain or a portion thereof is encoded by a polynucleotide comprising, consisting essentially of or consisting of a nucleic acid sequence of SEQ ID NO: 22.

In some aspects, the IL21R intracellular domain or a portion thereof comprises, consists essentially of or consists of an amino acid sequence at least 95%, 96%, 97%, 98%, 99% or 100% (or any percentage in between) identical to: SLKTHPLWRLWKKIWAVPSPERFFMPLYKGCSGDFKKWVGAPFTGSSLELGPWSPEVPSTLE VYSCHPPRSPAKRLQLTELQEPAELVESDGVPKPSFWPTAQNSGGSAYSEERDRPYGLVSID TVTVLDAEGPCTWPCSCEDDGYPALDLDAGLEPSPGLEDPLLDAGTTVLSCGCVSAGSPGLG GPLGSLLDRLKPPLADGEDWAGGLPWGGRSPGGVSESEAGSPLAGLDMDTFDSGFVGSDCSS PVECDFTSPGDEGPPRSYLRQWVVIPPPLSSPGPQAS (SEQ ID NO: 23). In a preferred aspect, the IL21R intracellular domain or a portion thereof comprises, consists essentially of, or consists of the amino acid sequence of SEQ ID NO: 23.

In some aspects, the IL21R intracellular domain or a portion thereof is encoded by a polynucleotide comprising, consisting essentially of or consisting of a nucleic acid sequence at least 95%, 96%, 97%, 98%, 99% or 100% (or any percentage in between) identical to:

(SEQ ID NO: 24)
AGCCTGAAAACACACCCACTCTGGCGGCTGTGGAAGAAAATCTGGGCCG
TGCCATCTCCTGAGCGGTTCTTCATGCCTCTGTACAAGGGCTGCAGCGG
CGACTTCAAGAAATGGGTCGGAGCCCCTTTTACCGGCAGCTCTCTGGAA
CTTGGACCTTGGAGCCCTGAGGTGCCCAGCACACTGGAAGTGTACAGCT
GTCACCCTCCTAGAAGCCCCGCCAAGAGACTGCAGCTGACAGAGCTGCA
AGAGCCTGCCGAGCTGGTGGAATCTGATGGCGTGCCCAAGCCTAGCTTC
TGGCCCACAGCTCAGAATAGCGGCGGCTCTGCCTACAGCGAGGAAAGGG
ATAGACCTTACGGCCTGGTGTCTATCGACACCGTGACCGTGCTGGATGC
CGAGGGACCTTGTACATGGCCTTGCAGCTGCGAGGACGATGGCTACCCT
GCTCTGGATCTGGACGCAGGCCTTGAGCCTTCTCCAGGACTGGAAGATC
CTCTGCTGGACGCCGGAACAACCGTGCTGTCTTGTGGCTGTGTGTCTGC
CGGATCTCCTGGACTTGGAGGCCCTCTGGGAAGCCTGCTGGATAGACTG
AAACCTCCTCTGGCCGACGGCGAAGATTGGGCTGGTGGACTTCCTTGGG
GCGGAAGATCTCCAGGCGGAGTGTCTGAGTCTGAAGCCGGTTCTCCACT
GGCCGGCCTGGACATGGATACCTTCGATTCTGGCTTCGTGGGCAGCGAC
TGTAGCAGCCCTGTGGAATGCGACTTCACAAGCCCTGGCGACGAGGGCC
CACCTAGAAGCTATCTGAGACAGTGGGTCGTGATCCCTCCACCTCTGTC
TAGTCCTGGACCTCAGGCCAGC.

In a preferred aspect, the IL21R intracellular domain or a portion thereof is encoded by a polynucleotide comprising, consisting essentially of or consisting of a nucleic acid sequence of SEQ ID NO: 24.

In some aspects, the ICOS intracellular domain or a portion thereof comprises, consists essentially of or consists of an amino acid sequence at least 95%, 96%, 97%, 98%, 99% or 100% (or any percentage in between) identical to: CWLTKKKYSSSVHDPNGEYMFMRAVNTAKKSRLTDVTL (SEQ ID NO: 25). In a preferred aspect, the ICOS intracellular domain or a portion thereof comprises, consists essentially of, or consists of the amino acid sequence of SEQ ID NO: 25.

In some aspects, the ICOS intracellular domain or a portion thereof is encoded by a polynucleotide comprising, consisting essentially of or consisting of a nucleic acid sequence at least 95%, 96%, 97%, 98%, 99% or 100% (or any percentage in between) identical to: TGTTGGCTGACCAAGAAAAAGTACAGCAGCAGCGTGCACGACCCCAACGGCGAGTACATGTT CATGAGAGCCGTGAACACCGCCAAGAAGTCCAGACTGACCGACGTGACCCTG (SEQ ID NO: 26). In a preferred aspect, the ICOS intracellular domain or a portion thereof is encoded by a polynucleotide comprising, consisting essentially of or consisting of a nucleic acid sequence of SEQ ID NO 26.

In some aspects, the CD27 intracellular domain or a portion thereof comprises, consists essentially of or consists of an amino acid sequence at least 95%, 96%, 97%, 98%, 99% or 100% (or any percentage in between) identical to: QRRKYRSNKGESPVEPAEPCHYSCPREEEGSTIPIQEDYRKPEPACSP (SEQ ID NO: 27). In a preferred aspect, the CD27 intracellular domain or a portion thereof comprises, consists essentially of, or consists of the amino acid sequence of SEQ ID NO: 27.

In some aspects, the CD27 intracellular domain or a portion thereof is encoded by a polynucleotide comprising, consisting essentially of or consisting of a nucleic acid sequence at least 95%, 96%, 97%, 98%, 99% or 100% (or any percentage in between) identical to: CAGCGGCGGAAGTACAGAAGCAACAAGGGCGAGAGCCCCGTGGAACCTGCCGAGCCTTGTCA CTACAGCTGCCCCAGAGAGGAAGAGGGCAGCACAATCCCCATCCAAGAGGACTACAGAAAGC CCGAGCCTGCCTGCTCTCCC (SEQ ID NO: 28). In a preferred aspect, the CD27 intracellular domain or a portion thereof is encoded by a polynucleotide comprising, consisting essentially of or consisting of a nucleic acid sequence of SEQ ID NO: 28.

In some aspects, the OX40 intracellular domain or a portion thereof comprises, consists essentially of or consists of an amino acid sequence at least 95%, 96%, 97%, 98%, 99% or 100% (or any percentage in between) identical to: ALYLLRRDQRLPPDAHKPPGGGSFRTPIQEEQADAHSTLAKI (SEQ ID NO: 29). In a preferred aspect, the OX40 intracellular domain or a portion thereof comprises, consists essentially of, or consists of the amino acid sequence of SEQ ID NO: 29.

In some aspects, the OX40 intracellular domain or a portion thereof is encoded by a polynucleotide comprising, consisting essentially of or consisting of a nucleic acid sequence at least 95%, 96%, 97%, 98%, 99% or 100% (or any percentage in between) identical to: GCCCTGTACCTGCTGCGGCGGGATCAAAGATTGCCTCCTGACGCTCACAAGCCTCCAGGCGG AGGCAGCTTTAGAACCCCTATCCAAGAGGAACAGGCTGACGCCCACAGCACCCTGGCCAAGA TC (SEQ ID NO 30). In a preferred aspect, the OX40 intracellular domain or a portion thereof is encoded by a polynucleotide comprising, consisting essentially of or consisting of a nucleic acid sequence of SEQ ID NO 30.

In some aspects, the GITR intracellular domain or a portion thereof comprises, consists essentially of or consists of an amino acid sequence at least 95%, 96%, 97%, 98%, 99% or 100% (or any percentage in between) identical to: QLGLHIWQLRSQCMWPRETQLLLEVPPSTEDARSCQFPEEERGERSAEEKGRLGDLWV (SEQ ID NO: 31). In a preferred aspect, the GITR intracellular domain or a portion thereof comprises, consists essentially of, or consists of the amino acid sequence of SEQ ID NO: 31.

In some aspects, the GITR intracellular domain or a portion thereof is encoded by a polynucleotide comprising, consisting essentially of or consisting of a nucleic acid sequence at least 95%, 96%, 97%, 98%, 99% or 100% (or any percentage in between) identical to: CAGCTGGGACTGCACATCTGGCAGCTGAGAAGCCAGTGCATGTGGCCCAGAGAGACACAGCT GCTGCTGGAAGTGCCTCCTAGCACCGAGGATGCCAGAAGCTGTCAGTTCCCCGAGGAAGAGA GAGGCGAGAGATCCGCCGAGGAAAAAGGCAGACTGGGCGACCTGTGGGTCCGAGTG (SEQ ID NO: 32). In a preferred aspect, the GITR intracellular domain or a portion thereof is encoded by a polynucleotide comprising, consisting essentially of or consisting of a nucleic acid sequence of SEQ ID NO: 32.

An exemplary “CSR CD2z” polypeptide of the disclosure comprises, consists essentially or consists of the amino acid sequence of (CD2 Signal peptide, CD2 Extracellular Domain, CD2 Transmembrane domain, CD 2 Cytoplasmic Domain, CD3ζ Intracellular Domain):

(SEQ ID NO: 33)
MSFPCKFVASFLLIFNVSSKGAVSKEITNALETWGALGQDTNLDTPSF
QMSDDIDDIKWEKTSDKKKIAQFRKEKETFKEKDTYKLFKNGTLKIKH
LKTDDQDIYKVSIYHTKGKNVLEKIFDLKIQERVSKPKISWTCINTTL
TCEVMNGTDPELNLYQDGKHLKLSQRVITHKWTTSLSAKFKCTAGNKV
SKESSVEPVSCPEKGLDIYLIIGICGGGSLLMVFVALLVFYIT

[symbol]

[symbol]

[symbol]

[symbol]

[symbol]

[symbol]

RVKFSRSADAPAYKQGQNQLYNELNLGRREEYDVLDKRRGRDPEMGG
KPRRKNPQEGLYNELQKDKMAEAYSEIGMKGERRRGKGHDGLYQGLS
TATKDTYDALHMQALPPR
CD2 Signal peptide: SEQ ID NO: 5
CD2 Extracellular Domain: SEQ ID NO: 1
CD2 Transmembrane domain: SEQ ID NO: 9
CD2 Cytoplasmic Domain: SEQ ID NO: 13
CD3ζ Intracellular Domain: SEQ ID NO: 11

An exemplary polynucleotide sequence encoding a “CSR CD2z” polypeptide of the disclosure comprises, consists essentially of or consists of the nucleic acid sequence of (CD2 Signal peptide, CD2 Extracellular Domain, C̅D̅2̅ T̅r̅a̅n̅s̅m̅e̅m̅b̅r̅a̅n̅e̅ d̅o̅m̅a̅i̅n̅, CD2Cytoplasmic Domain, Intracellular Domain):

(SEQ ID NO: 34)
ATGAGCTTCCCTTGCAAGTTCGTGGCCAGCTTCCTGCTGATCTTCAACGT
GTCCTCTAAGGGCGCCGTGTCCAAAGAGATCACAAACGCCCTGGAAACCT
GGGGAGCCCTCGGCCAGGATATTAACCTGGACATCCCCAGCTTCCAGATG
AGCGACGACATCGATGACATCAAGTGGGAGAAAACCAGCGACAAGAAGAA
GATCGCCCAGTTCCGGAAAGAGAAAGAGACATTCAAAGAGAAGGACACCT
ACAAGCTGTTCAAGAACGGCACCCTGAAGATCAAGCACCTGAAAACCGAC
GACCAGGACATCTATAAGGTGTCCATCTACGACACCAAGGGCAAGAACGT
GCTGGAAAAGATCTTCGACCTCAAGATCCAAGAGCGGGTGTCCAAGCCTA
AGATCAGCTGGACCTGCATCAACACCACACTGACCTGCGAAGTGATGAAC
GGCACAGACCCCGAGCTGAACCTGTACCAGGATGGCAAACACCTGAAGCT
GAGCCAGCGCGTGATCACCCACAAGTGGACAACAAGCCTGAGCGCCAAGT
TCAAGTGCACCGCCGGAAACAAAGTGTCTAAAGAGTCCAGCGTCGAGCCC
GTGTCTTGCCCTGAAAAAGGACTGGACATCTACCTGATCATCGGCATCTG
TGGCGGCGGAAGCCTGCTGATGGTGTTTGTGGCTCTGCTGGTGTTCTACA
TCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GAGTGAAGTTCAGCCGCAGCGCCGATGCTCC
TGCCTATAAGCAGGGACAGAACCAGCTGTACAACGAGCTGAATCTGGGGCG
CAGAGAAGAGTACGATGTGCTGGACAAGCGGAGAGGCAGAGATCCTGAGAT
GGGCGGCAAGCCCAGACGGAAGAATCCTCAAGAGGGCCTGTATAATGAGCT
GCAGAAAGACAAGATGGCCGAGGCCTACAGCGAGATCGGAATGAAGGGCGA
GCGCAGAAGAGGCAAGGGACACGATGGACTGTATCAGGGCCTGAGCACCGC
CACCAAGGATACCTATGATGCCCTGCACATGCAGGCCCTGCCTCCAAGA
CD2 Signal peptide: SEQ ID NO: 6
CD2 Extracellular Domain: SEQ ID NO: 2
CD2 Transmembrane domain: SEQ ID NO: 10
CD2 Cytoplasmic Domain: SEQ ID NO: 14
CD3ζ Intracellular Domain: SEQ ID NO: 12

An exemplary “mutant CSR CD2z-D111HT” or “CD2.DH.z” or “CSR 01 CD2.DH.z” polypeptide of the disclosure comprises or consists of the amino acid sequence of (CD2 Signal peptide, CD2 Extracellular domain with D111H mutation within the CD2 Extracellular domain, CD2 Transmembrane domain, CD2 Cytoplasmic domain, CD3ζ Intracellular domain):

(SEQ ID NO: 35)
MSFPCKFVASFLLIFNVSSKGAVSKEITNALETWGALGQDINLDTPSFQM
SDDIDDIKWEKTSDKKKIAQFRKEKETFKEKDTYKLFKNGTLKIKHLKTD
DQDIYKVSTYHTKGKNVLEKIFDLKIQERVSKPKISWTCINTTLTCEVMN
GTDPELNLYQDGKHLKLSQRVITHKWTTSLSAKFKCTAGNKVSKESSVEP
VSCPEKGLDIYLIIGICGGGSLLMVFVALLVFYIT

[symbol]

[symbol]

[symbol]

[symbol]

[symbol]

[symbol]

RVKFSRSADAPAYKQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR
CD2 Signal peptide: SEQ ID NO: 5
CD2 Extracellular domain with D111H mutation
within the CD2 Extracellular domain: SEQ ID NO: 3
CD2 Transmembrane domain: SEQ ID NO: 9
CD2 Cytoplasmic domain: SEQ ID NO: 13
CD3ζ Intracellular domain: SEQ ID NO: 11

An exemplary polynucleotide sequence encoding “mutant CSR CD2z-D111H” or “CD2.DH.z” or “CSR 01 CD2.DH.z” polypeptide of the disclosure comprises or consists of the amino acid sequence of (CD2 Signal peptide, CD2 Extracellular domain with D111H mutation within the CD2 Extracellular domain, CD2 Transmembrane domain, CD2 Cytoplasmic domain, CD3ζ Intracellular domain):

(SEQ ID NO: 36)
ATGAGCTTCCCTTGCAAGTTCGTGGCCAGCTTCCTGCTGATCTTCAACGT
GTCCTCTAAGGGCGCCGTGTCCAAAGAGATCACAAACGCCCTGGAAACC
TGGGGAGCCCTCGGCCAGGATATTAACCTGGACATCCCCAGCTTCCAGA
TGAGCGACGACATCGATGACATCAAGTGGGAGAAAACCAGCGACAAGAA
GAAGATCGCCCAGTTCCGGAAAGAGAAAGAGACATTCAAAGAGAAGGAC
ACCTACAAGCTGTTCAAGAACGGCACCCTGAAGATCAAGCACCTGAAAA
CCGACGACCAGGACATCTATAAGGTGTCCATCTACCACACCAAGGGCAA
GAACGTGCTGGAAAAGATCTTCGACCTCAAGATCCAAGAGCGGGTGTCC
AAGCCTAAGATCAGCTGGACCTGCATCAACACCACACTGACCTGCGAAG
TGATGAACGGCACAGACCCCGAGCTGAACCTGTACCAGGATGGCAAACA
CCTGAAGCTGAGCCAGCGCGTGATCACCCACAAGTGGACAACAAGCCTG
AGCGCCAAGTTCAAGTGCACCGCCGGAAACAAAGTGTCTAAAGAGTCCA
GCGTCGAGCCCGTGTCTTGCCCTGAAAAAGGACTGGACATCTACCTGAT
CATCGGCATCTGTGGCGGCGGAAGCCTGCTGATGGTGTTTGTGGCTCTG
CTGGTGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

A
GAGTGAAGTTCAGCCGCAGCGCCGATGCTCCTGCCTATAAGCAGGGACA
GAACCAGCTGTACAACGAGCTGATCTGGGGCGCAGAGAAGAGTACGATG
TGCTGGACAAGCGGAGAGGCAGAGATCCTGAGATGGGCGGCAAGCCCAG
ACGGAAGAATCCTCAAGAGGGCCTGTATAATGAGCTGCAGAAAGACAAG
ATGGCCGAGGCCTACAGCGAGATCGGAATGAAGGGCGAGCGCAGAAGAG
GCAAGGGACACGATGGACTGTATCAGGGCCTGAGCACCGCCACCAAGGA
TACCTATGATGCCCTGCACATGCAGGCCCTGCCTCCAAGA
CD2 Signal peptide: SEQ ID NO: 6
CD2 Extracellular domain with D111H mutation
within the CD2 Extracellular domain: SEQ ID NO: 4
CD2 Transmembrane domain: SEQ ID NO: 10
CD2 Cytoplasmic domain: SEQ ID NO: 14
CD3ζ Intracellular domain: SEQ ID NO: 12

An exemplary “mutant CSR 01b CD2.8.DH.z” or “CD2.8.DH.z” polypeptide of the disclosure comprises or consists of the amino acid sequence of (CD8a Signal peptide, CD2 Extracellular domain with D111H mutation within the CD2 Extracellular domain, CD2 Transmembrane domain, CD2 Cytoplasmic domain, CD3ζ Intracellular domain):

(SEQ ID NO: 37)
MALPVTALLLPLALLLHAARPKEITNALETWGALGQDINLDTPSFQMSD
DIDDIKWEKTSDKKKIAQFRKEKETFKEKDTYKLFKNGTLKIKHLKTDD
QDIYKVSIYHTKGKNVLEKIFDLKIQERVSKPKISWTCINTTLTCEVMN
GTDPELNLYQDGKHLKLSQRVITHKWTTSLSAKFKCTAGNKVSKESSVE
PVSCPEKGLDIYLIIGICGGGSLLMVFVALLVFYIT

[symbol]

[symbol]

[symbol]

[symbol]

[symbol]

[symbol]

RVKFSRSADA
PAYKQGQNQLYNELNLGRREEYDVLDKRRGRDPEMGGKPRRKNPQEGLY
NELQKDKMAEAYSETGMKGERRRGKGHDGLYQGLSTATKDTYDALHMQA
LPPR
CD8a Signal peptide: SEQ ID NO: 7
CD2 Extracellular domain with D111H mutation
within the CD2 Extracellular domain: SEQ ID NO: 3
CD2 Transmembrane domain: SEQ ID NO: 9
CD2 Intracellular domain: SEQ ID NO: 13
CD3ζ Intracellular domain: SEQ ID NO: 11

An exemplary polynucleotide sequence encoding “mutant CSR 01b CD2.8.DH.z” or “CD2.8.DH.z” polypeptide of the disclosure comprises or consists of the nucleic acid sequence of (CD8a Signal peptide, CD2 Extracellular domain with D111H mutation within the CD2 Extracellular domain, CD2 Transmembrane domain, CD2 Cytoplasmic domain, CD3ζ Intracellular domain):

(SEQ ID NO: 38)
ATGGCTCTGCCTGTGACAGCTCTGCTGCTGCCTCTGGCTCTGCTTCTGC
ATGCCGCCAGACCTAAAGAAATCACGAATGCATTGGAAACCTGGGGAGC
CCTCGGCCAGGATATTAACCTGGACATCCCCAGCTTCCAGATGAGCGAC
GACATCGATGACATCAAGTGGGAGAAAACCAGCGACAAGAAGAAGATCG
CCCAGTTCCGGAAAGAGAAAGAGACATTCAAAGAGAAGGACACCTACAA
GCTGTTCAAGAACGGCACCCTGAAGATCAAGCACCTGAAAACCGACGAC
CAGGACATCTATAAGGTGTCCATCTACCACACCAAGGGCAAGAACGTGC
TGGAAAAGATCTTCGACCTCAAGATCCAAGAGCGGGTGTCCAAGCCTAA
GATCAGCTGGACCTGCATCAACACCACACTGACCTGCGAAGTGATGAAC
GGCACAGACCCCGAGCTGAACCTGTACCAGGATGGCAAACACCTGAAGC
TGAGCCAGCGCGTGATCACCCACAAGTGGACAACAAGCCTGAGCGCCAA
GTTCAAGTGCACCGCCGGAAACAAAGTGTCTAAAGAGTCCAGCGTCGAG
CCCGTGTCTTGCCCTGAAAAAGGACTGGACATCTACCTGATCATCGGCA
TCTGTGGCGGCGGATCCCTGCTGATGGTGTTTGTGGCTCTGCTGGTGTT
CTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAATTCAGCCG
CAGCGCCGATGCTCCTGCCTATAAGCAGGGACAGAACCAGCTGTACAAC
GAGCTGAATCTGGGGCGCAGAGAAGAGTACGATGTGCTGGACAAGCGGA
GAGGCAGAGATCCTGAGATGGGCGGCAAGCCCAGACGGAAGAATCCTCA
AGAGGGCCTGTATAATGAGCTGCAGAAAGACAAGATGGCCGAGGCCTAC
AGCGAGATCGGAATGAAGGGCGAGCGCAGAAGAGGCAAGGGACACGATG
GACTGTATCAGGGCCTGAGCACCGCCACCAAGGATACCTATGATGCCCT
GCACATGCAGGCCCTGCCTCCAAGA
CD8a Signal peptide: SEQ ID NO: 8
CD2 Extracellular domain with D111H mutation
within the CD2 Extracellular domain: SEQ ID NO: 4
CD2 Transmembrane domain: SEQ ID NO: 10
CD2 Intracellular domain: SEQ ID NO: 14
CD3ζ Intracellular domain: SEQ ID NO: 12

An exemplary “mutant CSR 02 CD2.DH.28z” or “CD2.DH.28.z” polypeptide of the disclosure comprises or consists of the amino acid sequence of (CD2 Signal peptide, CD2 Extracellular domain with D111H mutation within the CD2 Extracellular domain, CD2 Transmembrane domain, CD2 Cytoplasmic domain, CD3ζ Intracellular domain):

(SEQ ID NO: 39)
MSFPCKFVASFLLIFNVSSKGAVSKEITNALETWGALGQDINLDIPSFQ
MSDDIDDIKWEKTSDKKKIAQFRKEKETFKEKDTYKLFKNGTLKIKHLK
TDDQDIYKVSIYHTKGKNVLEKIFDLKIQERVSKPKISWTCINTTLTCE
VMNGTDPELNLYQDGKHLKLSQRVITHKWTTSLSAKFKCTAGNKVSKES
SVEPVSCPEKGLDIYLIIGICGGGSLLMVFVALLVFYIT [symbol] [symbol] [symbol]
RVKFSRSA
DAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHM
QALPPR
CD2 Signal peptide: SEQ ID NO: 5
CD2 Extracellular domain with D111H mutation
within the CD2 Extracellular domain: SEQ ID NO: 3
CD2 Transmembrane domain: SEQ ID NO: 9
CD28 Intracellular domain: SEQ ID NO: 15
CD3ζ Intracellular domain: SEQ ID NO: 11

An exemplary polynucleotide sequence encoding a “mutant CSR 02 CD2.DH.28z” or “CD2.DH.28.z” polypeptide of the disclosure comprises or consists of the nucleic acid sequence of (CD2 Signal peptide, CD2 Extracellular domain with D111H mutation within the CD2 Extracellular domain, CD2 Transmembrane domain, CD2 Cytoplasmic domain, CD3ζ Intracellular domain):

(SEQ ID NO: 40)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

AGAGTGAAATTCAGCCGCAGCGCCGATGCTCCTGCCTATA
AGCAGGGACAGAACCAGCTGTACAACGAGCTGAATCTGGGGCGCAGAGAAGAGTACGATGTG
CTGGACAAGCGGAGAGGCAGAGATCCTGAGATGGGCGGCAAGCCCAGACGGAAGAATCCTCA
AGAGGGCCTGTATAATGAGCTGCAGAAAGACAAGATGGCCGAGGCCTACAGCGAGATCGGAA
TGAAGGGCGAGCGCAGAAGAGGCAAGGGACACGATGGACTGTATCAGGGCCTGAGCACCGCC
ACCAAGGATACCTATGATGCCCTGCACATGCAGGCCCTGCCTCCAAGA
CD2 Signal peptide: SEQ ID NO: 6
CD2 Extracellular domain with D111H mutation within
the CD2 Extracellular domain: SEQ
ID NO: 4
CD2 Transmembrane domain: SEQ ID NO: 10
CD28 Intracellular domain: SEQ ID NO: 16
CD3ζ Intracellular domain: SEQ ID NO: 12

An exemplary “mutant CSR 02b CD2.8.DH.28z” or “CD2.8.DH.28z” polypeptide of the disclosure comprises or consists of the amino acid sequence of (CD8a Signal peptide, CD2 Extracellular domain with D111H mutation within the CD2 Extracellular domain, CD2 Transmembrane domain, CD28 Intracellular domain, CD3ζ Intracellular domain):

(SEQ ID NO: 41)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol] [symbol]
RVKFSRSADAPAYKQGQNQLYNELNLGRREEYDVLDK
RRGRDPEMGGKPRRKNPQEGLYNELQKDKMAEAYSEIGMKGERRRGKGHDGLYQGLSTATKD
TYDALHMQALPPR
CD8a Signal peptide: SEQ ID NO: 7
CD2 Extracellular domain with D111H mutation within
the CD2 Extracellular domain: SEQ
ID NO: 3
CD2 Transmembrane domain: SEQ ID NO: 9
CD28 Intracellular domain: SEQ ID NO: 15
CD3ζ Intracellular domain: SEQ ID NO: 11

An exemplary polynucleotide acid sequence encoding a “mutant CSR 02b CD2.8.DH.28z” or “CD2.8.DH.28z” polypeptide of the disclosure comprises or consists of the nucleic acid sequence of (CD8a Signal peptide, CD2 Extracellular domain with D111H mutation within the CD2 Extracellular domain, CD2 Transmembrane domain, CD28 Intracellular domain, CD3ζ Intracellular domain):

(SEQ ID NO: 42)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

AGAGTGAAATTCAGCCGCAGCGCCGATGCTCCTGCCTATAAGCAGGGAC
AGAACCAGCTGTACAACGAGCTGAATCTGGGGCGCAGAGAAGAGTACGATGTGCTGGACAAG
CGGAGAGGCAGAGATCCTGAGATGGGCGGCAAGCCCAGACGGAAGAATCCTCAAGAGGGCCT
GTATAATGAGCTGCAGAAAGACAAGATGGCCGAGGCCTACAGCGAGATCGGAATGAAGGGCG
AGCGCAGAAGAGGCAAGGGACACGATGGACTGTATCAGGGCCTGAGCACCGCCACCAAGGAT
ACCTATGATGCCCTGCACATGCAGGCCCTGCCTCCAAGA
CD8a Signal peptide: SEQ ID NO: 8
CD2 Extracellular domain with D111H mutation within
the CD2 Extracellular domain: SEQ ID NO: 4
CD2 Transmembrane domain: SEQ ID NO: 10
CD28 Intracellular domain: SEQ ID NO: 16
CD3ζ Intracellular domain: SEQ ID NO: 12

An exemplary “mutant CSR 03 CD2.DH.BBz” or “CD2.DH.BBz” polypeptide of the disclosure comprises or consists of the amino acid sequence of (CD2 Signal peptide, CD2 Extracellular domain with D111H mutation within the CD2 Extracellular domain, CD2 Transmembrane domain, 4-1BB Intracellular domain, CD3ζ Intracellular domain):

(SEQ ID NO: 43)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQNQLYNELNLGRREEYD
VLDKRRGRDPEMGGKPRRKNPQEGLYNELQKDKMAEAYSEIGMKGERRRGKGHDGLYQGLST
ATKDTYDALHMQALPPR
CD2 Signal peptide: SEQ ID NO: 5
CD2 Extracellular domain with D111H mutation within
the CD2 Extracellular domain: SEQ
ID NO: 3
CD2 Transmembrane domain: SEQ ID NO: 9
4-1BB Intracellular domain: SEQ ID NO: 17
CD3ζ Intracellular domain: SEQ ID NO: 11

An exemplary polynucleotide sequence encoding a “mutant CSR 03 CD2.DH.BBz” or “CD2.DH.BBz” polypeptide of the disclosure comprises or consists of the nucleic acid sequence of (CD2 Signal peptide, CD2 Extracellular domain with D111H mutation within the CD2 Extracellular domain, CD2 Transmembrane domain, 4-1BB Intracellular domain, CD3ζ Intracellular domain):

(SEQ ID NO: 44)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

AGAGTGAAATTCAGCCGCAGCGCCGATGCTCCTGCCT
ATAAGCAGGGACAGAACCAGCTGTACAACGAGCTGATCTGGGGCGCAGAGAAGAGTACGAT
GTGCTGGACAAGCGGAGAGGCAGAGATCCTGAGATGGGCGGCAAGCCCAGACGGAAGAATCC
TCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTATCAGGGCCTGAGCACC
GCCACCAAGGATACCTATGATGCCCTGCACATGCAGGCCCTGCCTCCAAGA
CD2 Signal peptide: SEQ ID NO: 6
CD2 Extracellular domain with D111H mutation within
the CD2 Extracellular domain: SEQ
ID NO: 4
CD2 Transmembrane domain: SEQ ID NO: 10
4-1BB Intracellular domain: SEQ ID NO: 18
CD3ζ Intracellular domain: SEQ ID NO: 12

An exemplary “mutant CSR 03 CD2.8.DH.BBz” or “CD2.8.DH.BBz” polypeptide of the disclosure comprises or consists of the amino acid sequence of (CD8a Signal peptide, CD2 Extracellular domain with D111H mutation within the CD2 Extracellular domain, CD2 Transmembrane domain, 4-1BB Intracellular domain, CD3ζ Intracellular domain):

(SEQ ID NO: 45)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol] [symbol]
RVKFSRSADAPAYKQGQNQLYNELNLGRREEYDVLD
KRRGRDPEMGGKPRRKNPQEGLYNELQKDKMAEAYSEIGMKGERRRGKGHDGLYQGLSTATK
DTYDALHMQALPPR
CD8a Signal peptide: SEQ ID NO: 7
CD2 Extracellular domain with D111H mutation within
the CD2 Extracellular domain: SEQ
ID NO: 3
CD2 Transmembrane domain: SEQ ID NO: 9
4-1BB Intracellular domain: SEQ ID NO: 17
CD3ζ Intracellular domain: SEQ ID NO: 11

An exemplary polynucleotide acid sequence encoding a “mutant CSR 03b CD2.8.DH.BBz” or “CD2.8.DH.BBz” polypeptide of the disclosure comprises or consists of the nucleic acid sequence of (CD8a Signal peptide, CD2 Extracellular domain with D111H mutation within the CD2 Extracellular domain, CD2 Transmembrane domain, 4-1BB Intracellular domain, CD3ζ Intracellular domain):

(SEQ ID NO: 46)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
TGTTCTACATCACC

[symbol]

[symbol]

[symbol]

[symbol]

[symbol]

[symbol]

[symbol]

AGAGTGAAATTCAGCCGCAGCGCCGATGCTCCTGCCTATAAGCAGG
GACAGAACCAGCTGTACAACGAGCTGAATCTGGGGCGCAGAGAAGAGTACGATGTGCTGGAC
AAGCGGAGAGGCAGAGATCCTGAGATGGGCGGCAAGCCCAGACGGAAGAATCCTCAAGAGGG
CCTGTATAATGAGCTGCAGAAAGACAAGATGGCCGAGGCCTACAGCGAGATCGGAATGAAGG
GCGAGCGCAGAAGAGGCAAGGGACACGATGGACTGTATCAGGGCCTGAGCACCGCCACCAAG
GATACCTATGATGCCCTGCACATGCAGGCCCTGCCTCCAAGA
CD8a Signal peptide: SEQ ID NO: 8
CD2 Extracellular domain with D111H mutation within
the CD2 Extracellular domain: SEQ
ID NO: 4
CD2 Transmembrane domain: SEQ ID NO: 10
4-1BB Intracellular domain: SEQ ID NO: 18
CD3ζ Intracellular domain: SEQ ID NO: 12

An exemplary “mutant CSR 04 CD2.DH.7z” or “CD2.DH.7z” polypeptide of the disclosure comprises or consists of the amino acid sequence of (CD2 Signal peptide, CD2 Extracellular domain with D111H mutation within the CD2 Extracellular domain, CD2 Transmembrane domain, IL7RA Intracellular domain, CD3ζ Intracellular domain):

(SEQ ID NO: 47)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

RVKF
SRSADAPAYKQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
CD2 Signal peptide: SEQ ID NO: 5
CD2 Extracellular domain with D111H mutation within
the CD2 Extracellular domain: SEQ ID NO: 3
CD2 Transmembrane domain: SEQ ID NO: 9
IL7RA Intracellular domain: SEQ ID NO: 19
CD3ζ Intracellular domain: SEQ ID NO: 11

An exemplary polynucleotide acid sequence encoding a “mutant CSR 04 CD2.DH.7z” or “CD2.DH.7z” polypeptide of the disclosure comprises or consists of the nucleic acid sequence of (CD2 Signal peptide, CD2 Extracellular domain with D111H mutation within the CD2 Extracellular domain, CD2 Transmembrane domain, IL7RA Intracellular domain, CD3ζ Intracellular domain):

(SEQ ID NO: 48)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAATTC
AGCCGCAGCGCCGATGCTCCTGCCTATAAGCAGGGACAGAACCAGCTGTACAACGAGCTGAA
TCTGGGGCGCAGAGAAGAGTACGATGTGCTGGACAAGCGGAGAGGCAGAGATCCTGAGATGG
GCGGCAAGCCCAGACGGAAGAATCCTCAAGAGGGCCTGTATAATGAGCTGCAGAAAGACAAG
ATGGCCGAGGCCTACAGCGAGATCGGAATGAAGGGCGAGCGCAGAAGAGGCAAGGGACACGA
TGGACTGTATCAGGGCCTGAGCACCGCCACCAAGGATACCTATGATGCCCTGCACATGCAGG
CCCTGCCTCCAAGA
CD2 Signal peptide: SEQ ID NO: 6
CD2 Extracellular domain with D111H mutation within
the CD2 Extracellular domain: SEQ
ID NO: 4
CD2 Transmembrane domain: SEQ ID NO: 10
IL7RA Intracellular domain: SEQ ID NO: 20
CD3ζ Intracellular domain: SEQ ID NO: 12

An exemplary “mutant CSR 04b CD2.8.DH.7z” or “CD2.8.DH.7z” polypeptide of the disclosure comprises or consists of the amino acid sequence of (CD8a Signal peptide, CD2 Extracellular domain with D111H mutation within the CD2 Extracellular domain, CD2 Transmembrane domain, IL7RA Intracellular domain, CD3ζ Intracellular domain):

(SEQ ID NO: 49)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVERVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

RVKFSRS
ADAPAYKQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
CD8a Signal peptide: SEQ ID NO: 7
CD2 Extracellular domain with D111H mutation within
the CD2 Extracellular domain: SEQ ID NO: 3
CD2 Transmembrane domain: SEQ ID NO: 9
IL7RA Intracellular domain: SEQ ID NO: 19
CD3ζ Intracellular domain: SEQ ID NO: 11

An exemplary polynucleotide acid sequence encoding a “mutant CSR 04b CD2.8.DH.7z” or “CD2.8.DH.7z” polypeptide of the disclosure comprises or consists of the nucleic acid sequence of (CD8a Signal peptide, CD2 Extracellular domain with D111H mutation within the CD2 Extracellular domain, CD2 Transmembrane domain, IL7RA Intracellular domain, CD3ζ Intracellular domain):

(SEQ ID NO: 50)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAATTCAGCCGCAGC
GCCGATGCTCCTGCCTATAAGCAGGGACAGAACCAGCTGTACAACGAGCTGAATCTGGGGCG
CAGAGAAGAGTACGATGTGCTGGACAAGCGGAGAGGCAGAGATCCTGAGATGGGCGGCAAGC
CCAGACGGAAGAATCCTCAAGAGGGCCTGTATAATGAGCTGCAGAAAGACAAGATGGCCGAG
GCCTACAGCGAGATCGGAATGAAGGGCGAGCGCAGAAGAGGCAAGGGACACGATGGACTGTA
TCAGGGCCTGAGCACCGCCACCAAGGATACCTATGATGCCCTGCACATGCAGGCCCTGCCTC
CAAGA
CD8a Signal peptide: SEQ ID NO: 8
CD2 Extracellular domain with D111H mutation within
the CD2 Extracellular domain: SEQ
ID NO: 4
CD2 Transmembrane domain: SEQ ID NO: 10
IL7RA Intracellular domain: SEQ ID NO: 20
CD3ζ Intracellular domain: SEQ ID NO: 12

An exemplary “mutant CSR 05 CD2.DH.15z” or “CD2.DH.15z” polypeptide of the disclosure comprises or consists of the amino acid sequence of (CD2 Signal peptide, CD2 Extracellular domain with D111H mutation within the CD2 Extracellular domain, CD2 Transmembrane domain, IL15RA Intracellular domain, CD3ζ Intracellular domain):

(SEQ ID NO: 51)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQNQLYNELNLGRREEYDVLD
KRRGRDPEMGGKPRRKNPQEGLYNELQKDKMAEAYSEIGMKGERRRGKGHDGLYQGLSTATK
DTYDALHMQALPPR
CD2 Signal peptide: SEQ ID NO: 5
CD2 Extracellular domain with D111H mutation within
the CD2 Extracellular domain: SEQ ID NO: 3
CD2 Transmembrane domain: SEQ ID NO: 9
IL15RA Intracellular domain: SEQ ID NO: 21
CD3ζ Intracellular domain: SEQ ID NO: 11

An exemplary polynucleotide acid sequence encoding a “mutant CSR 05 CD2.DH.15z” or “CD2.DH.15z” polypeptide of the disclosure comprises or consists of the nucleic acid sequence of (CD2 Signal peptide, CD2 Extracellular domain with D111H mutation within the CD2 Extracellular domain, CD2 Transmembrane domain, IL15RA Intracellular domain, CD3ζ Intracellular domain):

(SEQ ID NO: 52)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

AGAGTGAAATTCAGCCGCAGCGCCGATGCTCCTGCCTATAAGCAGG
GACAGAACCAGCTGTACAACGAGCTGAATCTGGGGCGCAGAGAAGAGTACGATGTGCTGGAC
AAGCGGAGAGGCAGAGATCCTGAGATGGGCGGCAAGCCCAGACGGAAGAATCCTCAAGAGGG
CCTGTATAATGAGCTGCAGAAAGACAAGATGGCCGAGGCCTACAGCGAGATCGGAATGAAGG
GCGAGCGCAGAAGAGGCAAGGGACACGATGGACTGTATCAGGGCCTGAGCACCGCCACCAAG
GATACCTATGATGCCCTGCACATGCAGGCCCTGCCTCCAAGA
CD2 Signal peptide: SEQ ID NO: 6
CD2 Extracellular domain with D111H mutation within
the CD2 Extracellular domain: SEQ ID NO: 4
CD2 Transmembrane domain: SEQ ID NO: 10
IL15RA Intracellular domain: SEQ ID NO: 22
CD3ζ Intracellular domain: SEQ ID NO: 12

An exemplary “mutant CSR 05b CD2.8.DH.15z” or “CD2.8.DH.15z” polypeptide of the disclosure comprises or consists of the amino acid sequence of (CD8a Signal peptide, CD2 Extracellular domain with D111H mutation within the CD2 Extracellular domain, CD2 Transmembrane domain, IL15RA Intracellular domain, CD3ζ Intracellular domain):

(SEQ ID NO: 53)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol]
RVKFSRSADAPAYKQGQNQLYNELNLGRREEYDVLDKRR
GRDPEMGGKPRRKNPQEGLYNELQKDKMAEAYSEIGMKGERRRGKGHDGLYQGLSTATKDTY
DALHMQALPPR
CD8a Signal peptide: SEQ ID NO: 7
CD2 Extracellular domain with D111H mutation within
the CD2 Extracellular domain: SEQ ID NO: 3
CD2 Transmembrane domain: SEQ ID NO: 9
IL15RA Intracellular domain: SEQ ID NO: 21
CD3ζ Intracellular domain: SEQ ID NO: 11

An exemplary polynucleotide acid sequence encoding a “mutant CSR 05b CD2.8.DH.15z” or “CD2.8.DH.15z” polypeptide of the disclosure comprises or consists of the nucleic acid sequence of (CD8a Signal peptide, CD2 Extracellular domain with D111H mutation within the CD2 Extracellular domain, CD2 Transmembrane domain, IL15RA Intracellular domain, CD3ζ Intracellular domain):

(SEQ ID NO: 54)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

AGAGTGAAATTCAGCCGCAGCGCCGATGCTCCTGCCTATAAGCAGGGACAGAACC
AGCTGTACAACGAGCTGAATCTGGGGCGCAGAGAAGAGTACGATGTGCTGGACAAGCGGAGA
GGCAGAGATCCTGAGATGGGCGGCAAGCCCAGACGGAAGAATCCTCAAGAGGGCCTGTATAA
TGAGCTGCAGAAAGACAAGATGGCCGAGGCCTACAGCGAGATCGGAATGAAGGGCGAGCGCA
GAAGAGGCAAGGGACACGATGGACTGTATCAGGGCCTGAGCACCGCCACCAAGGATACCTAT
GATGCCCTGCACATGCAGGCCCTGCCTCCAAGA
CD8a Signal peptide: SEQ ID NO: 8
CD2 Extracellular domain with D111H mutation within
the CD2 Extracellular domain: SEQ
ID NO: 4
CD2 Transmembrane domain: SEQ ID NO: 10
IL15RA Intracellular domain: SEQ ID NO: 22
CD3ζ Intracellular domain: SEQ ID NO: 12

An exemplary “mutant CSR 06 CD2.DH.21z” or “CD2.DH.21z” polypeptide of the disclosure comprises or consists of the amino acid sequence of (CD2 Signal peptide, CD2 Extracellular domain with D111H mutation within the CD2 Extracellular domain, CD2 Transmembrane domain, IL21RA Intracellular domain, CD3ζ Intracellular domain):

(SEQ ID NO: 55)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

RVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDT
YDALHMQALPPR
CD2 Signal peptide: SEQ ID NO: 5
CD2 Extracellular domain with D111H mutation within the CD2 Extracellular domain:
SEQ ID NO: 3
CD2 Transmembrane domain: SEQ ID NO: 9
IL21R Intracellular domain: SEQ ID NO: 23
CD3ζ Intracellular domain: SEQ ID NO: 11

An exemplary polynucleotide acid sequence encoding “mutant CSR 06 CD2.DH.21z” or “CD2.DH.21z” polypeptide of the disclosure comprises or consists of the nucleic acid sequence of (CD2 Signal peptide, CD2 Extracellular domain with D111H mutation within the CD2 Extracellular domain, CD2 Transmembrane domain, IL21R Intracellular domain, CD3 Intracellular domain):

(SEQ ID NO: 56)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAATTCAGCCGCAGCGCCGATGCTCCTGCCTATAAGCAGGGACAGA
ACCAGCTGTACAACGAGCTGAATCTGGGGCGCAGAGAAGAGTACGATGTGCTGGACAAGCGG
AGAGGCAGAGATCCTGAGATGGGCGGCAAGCCCAGACGGAAGAATCCTCAAGAGGGCCTGTA
TAATGAGCTGCAGAAAGACAAGATGGCCGAGGCCTACAGCGAGATCGGAATGAAGGGCGAGC
GCAGAAGAGGCAAGGGACACGATGGACTGTATCAGGGCCTGAGCACCGCCACCAAGGATACC
TATGATGCCCTGCACATGCAGGCCCTGCCTCCAAGA
CD2 Signal peptide: SEQ ID NO: 6
CD2 Extracellular domain with D111H mutation within the CD2 Extracellular domain:
SEQ ID NO: 4
CD2 Transmembrane domain: SEQ ID NO: 10
IL21R Intracellular domain: SEQ ID NO: 24
CDζ Intracellular domain: SEQ ID NO: 12

An exemplary “mutant CSR 06b CD2.8.DH.21z” or “CD2.8.DH.21z” polypeptide of the disclosure comprises or consists of the amino acid sequence of (CD8a Signal peptide, CD2 Extracellular domain with D111H mutation within the CD2 Extracellular domain, CD2 Transmembrane domain, IL21R Intracellular domain, CD3ζ Intracellular domain):

(SEQ ID NO: 57)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT

[symbol]

[symbol]

[symbol]

[symbol]

[symbol]

[symbol]

[symbol]

[symbol]

[symbol]

[symbol]

[symbol]

[symbol]

[symbol]

[symbol]

RVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDTYDA
LHMQALPPR
CD8a Signal peptide: SEQ ID NO: 7
CD2 Extracellular domain with D111H mutation within the CD2 Extracellular domain:
SEQ ID NO: 3
CD2 Transmembrane domain: SEQ ID NO: 9
IL21R Intracellular domain: SEQ ID NO: 23
CDζ Intracellular domain: SEQ ID NO: 11

An exemplary polynucleotide acid sequence encoding “mutant CSR 06b CD2.8.DH.21z” or “CD2.8.DH.21z” polypeptide of the disclosure comprises or consists of the nucleic acid sequence of (CD8a Signal peptide, CD2 Extracellular domain with D111H mutation within the CD2 Extracellular domain, CD2 Transmembrane domain, IL21R Intracellular domain, CD3ζ Intracellular domain):

(SEQ ID NO: 58)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AGAGTGAAATTCAGCCGCAGCGCCGATGCTCCTGCCTATAAGCAGGGACAGAACCAGCTGT
ACAACGAGCTGATCTGGGGCGCAGAGAAGAGTACGATGTGCTGGACAAGCGGAGAGGCAGA
GATCCTGAGATGGGCGGCAAGCCCAGACGGAAGAATCCTCAAGAGGGCCTGTATAATGAGCT
GCAGAAAGACAAGATGGCCGAGGCCTACAGCGAGATCGGAATGAAGGGCGAGCGCAGAAGAG
GCAAGGGACACGATGGACTGTATCAGGGCCTGAGCACCGCCACCAAGGATACCTATGATGCC
CTGCACATGCAGGCCCTGCCTCCAAGA
CD8a Signal peptide: SEQ ID NO: 8
CD2 Extracellular domain with D111H mutation within the CD2 Extracellular domain:
SEQ ID NO: 4
CD2 Transmembrane domain: SEQ ID NO: 10
IL21R Intracellular domain: SEQ ID NO: 24
CDζ Intracellular domain: SEQ ID NO: 12

An exemplary “mutant CSR 07 CD2.DH.Iz” or “CD2.DH.Iz” polypeptide of the disclosure comprises or consists of the amino acid sequence of (CD2 Signal peptide, CD2 Extracellular domain with D111H mutation within the CD2 Extracellular domain, CD2 Transmembrane domain, ICOS Intracellular domain, CD3ζ Intracellular domain):

(SEQ ID NO: 59)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQNQLYNELNLGRREEYDVLDK
RRGRDPEMGGKPRRKNPQEGLYNELQKDKMAEAYSEIGMKGERRRGKGHDGLYQGLSTATKD
TYDALHMQALPPR
CD2 Signal peptide: SEQ ID NO: 5
CD2 Extracellular domain with D111H mutation within the CD2 Extracellular domain:
SEQ ID NO: 3
CD2 Transmembrane domain: SEQ ID NO: 9
ICOS Intracellular domain: SEQ ID NO: 25
CD3ζ Intracellular domain: SEQ ID NO: 11

An exemplary polynucleotide acid sequence encoding a “mutant CSR 07 CD2.DH.Iz” or “CD2.DH.Iz” polypeptide of the disclosure comprises or consists of the nucleic acid sequence of (CD2 Signal peptide, CD2 Extracellular domain with D111H mutation within the CD2 Extracellular domain, CD2 Transmembrane domain, ICOS Intracellular domain, CD3ζ Intracellular domain):

(SEQ ID NO: 60)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

AGAGTGAAATTCAGCCGCAGCGCCGATGCTCCTGCCTATAAGCAGGGAC
AGAACCAGCTGTACAACGAGCTGAATCTGGGGCGCAGAGAAGAGTACGATGTGCTGGACAAG
CGGAGAGGCAGAGATCCTGAGATGGGCGGCAAGCCCAGACGGAAGAATCCTCAAGAGGGCCT
GTATAATGAGCTGCAGAAAGACAAGATGGCCGAGGCCTACAGCGAGATCGGAATGAAGGGCG
AGCGCAGAAGAGGCAAGGGACACGATGGACTGTATCAGGGCCTGAGCACCGCCACCAAGGAT
ACCTATGATGCCCTGCACATGCAGGCCCTGCCTCCAAGA
CD2 Signal peptide: SEQ ID NO: 6
CD2 Extracellular domain with D111H mutation within the CD2 Extracellular domain:
SEQ ID NO: 4
CD2 Transmembrane domain: SEQ ID NO: 10
ICOS Intracellular domain: SEQ ID NO: 26
CD3ζ Intracellular domain: SEQ ID NO: 12

An exemplary “mutant CSR 07b CD2.8.DH.Iz” or “CD2.8.DH.Iz” polypeptide of the disclosure comprises or consists of the amino acid sequence of (CD8a Signal peptide, CD2 Extracellular domain with D111H mutation within the CD2 Extracellular domain, CD2 Transmembrane domain, ICOS Intracellular domain, CD3ζ Intracellular domain):

(SEQ ID NO: 61)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol]
RVKFSRSADAPAYKQGQNQLYNELNLGRREEYDVLDKRRG
RDPEMGGKPRRKNPQEGLYNELQKDKMAEAYSEIGMKGERRRGKGHDGLYQGLSTATKDTYD
ALHMQALPPR
CD8a Signal peptide: SEQ ID NO: 7
CD2 Extracellular domain with D111H mutation within the CD2 Extracellular domain:
SEQ ID NO: 3
CD2 Transmembrane domain: SEQ ID NO: 9
ICOS Intracellular domain: SEQ ID NO: 25
CD3ζ Intracellular domain: SEQ ID NO: 11

An exemplary polynucleotide acid sequence encoding a “mutant CSR 07b CD2.8.DH.Iz” or “CD2.8.DH.Iz” polypeptide of the disclosure comprises or consists of the nucleic acid sequence of (CD8a Signal peptide, CD2 Extracellular domain with D111H mutation within the CD2 Extracellular domain, CD2 Transmembrane domain, ICOS Intracellular domain, CD3ζ Intracellular domain):

(SEQ ID NO: 62)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

AGAGTGAAATTCAGCCGCAGCGCCGATGCTCCTGCCTATAAGCAGGGACAGAACCAGC
TGTACAACGAGCTGATCTGGGGCGCAGAGAAGAGTACGATGTGCTGGACAAGCGGAGAGGC
AGAGATCCTGAGATGGGCGGCAAGCCCAGACGGAAGAATCCTCAAGAGGGCCTGTATAATGA
GCTGCAGAAAGACAAGATGGCCGAGGCCTACAGCGAGATCGGAATGAAGGGCGAGCGCAGAA
GAGGCAAGGGACACGATGGACTGTATCAGGGCCTGAGCACCGCCACCAAGGATACCTATGAT
GCCCTGCACATGCAGGCCCTGCCTCCAAGA
CD8a Signal peptide: SEQ ID NO: 8
CD2 Extracellular domain with D111H mutation within
the CD2 Extracellular domain: SEQ ID NO: 4
CD2 Transmembrane domain: SEQ ID NO: 10
ICOS Intracellular domain: SEQ ID NO: 26
CD3ζ Intracellular domain: SEQ ID NO: 12

An exemplary “mutant CSR 08 CD2.DH.27z” or “CD2.DH.27z” polypeptide of the disclosure comprises or consists of the amino acid sequence of (CD2 Signal peptide, CD2 Extracellular domain with D111H mutation within the CD2 Extracellular domain, CD2 Transmembrane domain, CD27 Intracellular domain, CD3ζ Intracellular domain):

(SEQ ID NO: 63)
MSFPCKFVASFLLIFNVSSKGAVSKEITNALETWGA
LGQDINLDIPSFQMSDDIDDIKWEKTSDKKKIAQF
RKEKETFKEKDTYKLFKNGTLKIKHLKTDDQDIYK
VSIYHTKGKNVLEKIFDLKIQERVSKPKISWTCIN
TTLTCEVMNGTDPELNLYQDGKHLKLSQRVITHKW
TTSLSAKFKCTAGNKVSKESSVEPVSCPEKGLDIY
LIIGICGGGSLLMVFVALLVFYIT

[symbol]

[symbol]

[symbol]

RVKFSRSADAPAYKQGQNQLYNELNLGRREEYD
VLDKRRGRDPEMGGKPRRKNPQEGLYNELQKDKMA
EAYSEIGMKGERRRGKGHDGL
YQGLSTATKDTYDALHMQALPPR
CD2 Signal peptide: SEQ ID NO: 5
CD2 Extracellular domain with D111H
mutation within
the CD2 Extracellular domain: SEQ ID NO: 3
CD2 Transmembrane domain: SEQ ID NO: 9
CD27 Intracellular domain: SEQ ID NO: 27
CD3ζ Intracellular domain: SEQ ID NO: 11

An exemplary polynucleotide acid sequence encoding a “mutant CSR 08 CD2.DH.27z” or “CD2.DH.27z” polypeptide of the disclosure comprises or consists of the nucleic acid sequence of (CD2 Signal peptide, CD2 Extracellular domain with D111H mutation within the CD2 Extracellular domain, CD2 Transmembrane domain, CD27 Intracellular domain, CD3ζ Intracellular domain):

(SEQ ID NO: 64)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

AGAGTGAAATTCAGCCGCA
GCGCCGATGCTCCTGCCTATAAGCAGGGACAGAACCAGCTGTACAACGAGCTGAATCTGGGG
CGCAGAGAAGAGTACGATGTGCTGGACAAGCGGAGAGGCAGAGATCCTGAGATGGGCGGCAA
GCCCAGACGGAAGAATCCTCAAGAGGGCCTGTATAATGAGCTGCAGAAAGACAAGATGGCCG
AGGCCTACAGCGAGATCGGAATGAAGGGCGAGCGCAGAAGAGGCAAGGGACACGATGGACTG
TATCAGGGCCTGAGCACCGCCACCAAGGATACCTATGATGCCCTGCACATGCAGGCCCTGCC
TCCAAGA
CD2 Signal peptide: SEQ ID NO: 6
CD2 Extracellular domain with D111H mutation within
the CD2 Extracellular domain: SEQ ID NO: 4
CD2 Transmembrane domain: SEQ ID NO: 10
CD27 Intracellular domain: SEQ ID NO:
CD3ζ Intracellular domain: SEQ ID NO:

An exemplary “mutant CSR 08b CD2.8.DH.27z” or “CD2.8.DH.27z” polypeptide of the disclosure comprises or consists of the amino acid sequence of (CD8a Signal peptide, CD2 Extracellular domain with D111H mutation within the CD2 Extracellular domain, CD2 Transmembrane domain, CD27 Intracellular domain, CD3ζ Intracellular domain):

(SEQ ID NO: 65)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol] [symbol]
RVKFSRSADAPAYKQGQNQLYNELNLGRRE
EYDVLDKRRGRDPEMGGKPRRKNPQEGLYNELQKDKMAEAYSEIGMKGERRRGKGHDGLYQG
LSTATKDTYDALHMQALPPR
CD8a Signal peptide: SEQ ID NO: 7
CD2 Extracellular domain with D111H mutation within
the CD2 Extracellular domain: SEQ ID NO: 3
CD2 Transmembrane domain: SEQ ID NO: 9
CD27 Intracellular domain: SEQ ID NO: 27
CD3ζ Intracellular domain: SEQ ID NO: 11

An exemplary polynucleotide acid sequence encoding a “mutant CSR 08b CD2.8.DH.27z” or “CD2.8.DH.27z” polypeptide of the disclosure comprises or consists of the nucleic acid sequence of (CD8a Signal peptide, CD2 Extracellular domain with D111H mutation within the CD2 Extracellular domain, CD2 Transmembrane domain, CD27 Intracellular domain, CD3ζ Intracellular domain):

(SEQ ID NO: 66)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

AGAGTGAAATTCAGCCGCAGCGCCGATG
CTCCTGCCTATAAGCAGGGACAGAACCAGCTGTACAACGAGCTGAATCTGGGGCGCAGAGAA
GAGTACGATGTGCTGGACAAGCGGAGAGGCAGAGATCCTGAGATGGGCGGCAAGCCCAGACG
GAAGAATCCTCAAGAGGGCCTGTATAATGAGCTGCAGAAAGACAAGATGGCCGAGGCCTACA
GCGAGATCGGAATGAAGGGCGAGCGCAGAAGAGGCAAGGGACACGATGGACTGTATCAGGGC
CTGAGCACCGCCACCAAGGATACCTATGATGCCCTGCACATGCAGGCCCTGCCTCCAAGA
CD8a Signal peptide: SEQ ID NO: 8
CD2 Extracellular domain with D111H mutation
within the CD2 Extracellular domain: SEQ ID NO: 4
CD2 Transmembrane domain: SEQ ID NO: 10
CD27 Intracellular domain: SEQ ID NO: 28
CD3 Intracellular domain: SEQ ID NO: 12

An exemplary “mutant CSR 09 CD2.DH.Oxz” or “CD2.DH.Oxz” polypeptide of the disclosure comprises or consists of the amino acid sequence of (CD2 Signal peptide, CD2 Extracellular domain with D111H mutation within the CD2 Extracellular domain, CD2 Transmembrane domain, OX40 Intracellular domain, CD3ζ Intracellular domain):

(SEQ ID NO: 67)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQNQLYNELNLGRREEYD
VLDKRRGRDPEMGGKPRRKNPQEGLYNELQKDKMAEAYSEIGMKGERRRGKGHDGLYQGLST
ATKDTYDALHMQALPPR
CD2 Signal peptide: SEQ ID NO: 5
CD2 Extracellular domain with D111H mutation within
the CD2 Extracellular domain: SEQ ID NO: 3
CD2 Transmembrane domain: SEQ ID NO: 9
OX40 Intracellular domain: SEQ ID NO: 29
CD3ζ Intracellular domain: SEQ ID NO: 11

An exemplary polynucleotide acid sequence encoding a “mutant CSR 09 CD2.DH.Oxz” or “CD2.DH.Oxz” polypeptide of the disclosure comprises or consists of the nucleic acid sequence of (CD2 Signal peptide, CD2 Extracellular domain with D111H mutation within the CD2 Extracellular domain, CD2 Transmembrane domain, OX40 Intracellular domain, CD3ζ Intracellular domain):

(SEQ ID NO: 68)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACC

[symbol]

[symbol]

[symbol]

[symbol]

[symbol]

[symbol]

[symbol]

AGAGTGAAATTCAGCCGCAGCGCCGATGCTCCTGCCT
ATAAGCAGGGACAGAACCAGCTGTACAACGAGCTGAATCTGGGGCGCAGAGAAGAGTACGAT
GTGCTGGACAAGCGGAGAGGCAGAGATCCTGAGATGGGCGGCAAGCCCAGACGGAAGAATCC
TCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTATCAGGGCCTGAGCACC
GCCACCAAGGATACCTATGATGCCCTGCACATGCAGGCCCTGCCTCCAAGA
CD2 Signal peptide:
SEQ ID NO: 6
CD2 Extracellular domain with D111H mutation within
the CD2 Extracellular domain:
SEQ ID NO: 4
CD2 Transmembrane domain:
SEQ ID NO: 10
OX40 Intracellular domain:
SEQ ID NO: 30
CD3ζ Intracellular domain:
SEQ ID NO: 12

An exemplary “mutant CSR 09b CD2.8.DH.Oxz” or “CD2.8.DH.Oxz” polypeptide of the disclosure comprises or consists of the amino acid sequence of (CD8a Signal peptide, CD2 Extracellular domain with D111H mutation within the CD2 Extracellular domain, CD2 Transmembrane domain, OX40 Intracellular domain, CD3ζ Intracellular domain):

(SEQ ID NO: 69)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol] [symbol]
RVKFSRSADAPAYKQGQNQLYNELNLGRREEYDVLD
KRRGRDPEMGGKPRRKNPQEGLYNELQKDKMAEAYSEIGMKGERRRGKGHDGLYQGLSTATK
DTYDALHMQALPPR
CD8a Signal peptide: SEQ ID NO: 7
CD2 Extracellular domain with D111H mutation within
the CD2 Extracellular domain: SEQ ID NO: 3
CD2 Transmembrane domain: SEQ ID NO: 9
OX40 Intracellular domain: SEQ ID NO: 29
CD3ζ Intracellular domain: SEQ ID NO: 11

An exemplary polynucleotide acid sequence encoding a “mutant CSR 09b CD2.8.DH.Oxz” or “CD2.8.DH.Oxz” polypeptide of the disclosure comprises or consists of the nucleic acid sequence of (CD8a Signal peptide, CD2 Extracellular domain with D111H mutation within the CD2 Extracellular domain, CD2 Transmembrane domain, OX40 Intracellular domain, CD3ζ Intracellular domain):

(SEQ ID NO: 70)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACC

[symbol]

[symbol]

[symbol]

[symbol]

[symbol]

[symbol]

[symbol]

AGAGTGAAATTCAGCCGCAGCGCCGATGCTCCTGCCTATAAGCAGG
GACAGAACCAGCTGTACAACGAGCTGAATCTGGGGCGCAGAGAAGAGTACGATGTGCTGGAC
AAGCGGAGAGGCAGAGATCCTGAGATGGGCGGCAAGCCCAGACGGAAGAATCCTCAAGAGGG
CCTGTATAATGAGCTGCAGAAAGACAAGATGGCCGAGGCCTACAGCGAGATCGGAATGAAGG
GCGAGCGCAGAAGAGGCAAGGGACACGATGGACTGTATCAGGGCCTGAGCACCGCCACCAAG
GATACCTATGATGCCCTGCACATGCAGGCCCTGCCTCCAAGA
CD8a Signal peptide: SEQ ID NO: 8
CD2 Extracellular domain with D111H mutation
within the CD2 Extracellular domain: SEQ ID NO: 4
CD2 Transmembrane domain: SEQ ID NO: 10
OX40 Intracellular domain: SEQ ID NO: 30
CD3ζ Intracellular domain: SEQ ID NO: 12

An exemplary “mutant CSR 10 CD2.DH.Gz” or “CD2.DH.Gz” polypeptide of the disclosure comprises or consists of the amino acid sequence of (CD2 Signal peptide, CD2 Extracellular domain with D111H mutation within the CD2 Extracellular domain, CD2 Transmembrane domain, GITR Intracellular domain, CD3ζ Intracellular domain):

(SEQ ID NO: 71)
MSFPCKFVASFLLIFNVSSKGAVSKEITNALETWGALGQDINLDIPSFQMSDDIDDIKWEKT
SDKKKIAQFRKEKETFKEKDTYKLFKNGTLKIKHLKTDDQDIYKVSIYHTKGKNVLEKIFDL
KIQERVSKPKISWTCINTTLTCEVMNGTDPELNLYQDGKHLKLSQRVITHKWTTSLSAKFKC
TAGNKVSKESSVEPVSCPEKGLDIYLIIGICGGGSLLMVFVALLVFYIT [symbol] [symbol] [symbol]
RVKFSRSADAPAYKQGQ
NQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
CD2 Signal peptide: SEQ ID NO: 5
CD2 Extracellular domain with D111H mutation within
the CD2 Extracellular domain: SEQ ID NO: 3
CD2 Transmembrane domain: SEQ ID NO: 9
GITR Intracellular domain: SEQ ID NO: 31
CD3ζ Intracellular domain: SEQ ID NO: 11

An exemplary polynucleotide acid sequence encoding a “mutant CSR 10 CD2.DH.Gz” or “CD2.DH.Gz” polypeptide of the disclosure comprises or consists of the nucleic acid sequence of (CD2 Signal peptide, CD2 Extracellular domain with D111H mutation within the CD2 Extracellular domain, CD2 Transmembrane domain, GITR Intracellular domain, CD3ζ Intracellular domain):

(SEQ ID NO: 72)
ATGAGCTTCCCTTGCAAGTTCGTGGCCAGCTTCCTGCTGATCTTCAACGTGTCCTCTAAGGG
CGCCGTGTCCAAAGAAATCACGAATGCATTGGAAACCTGGGGAGCCCTCGGCCAGGATATTA
ACCTGGACATCCCCAGCTTCCAGATGAGCGACGACATCGATGACATCAAGTGGGAGAAAACC
AGCGACAAGAAGAAGATCGCCCAGTTCCGGAAAGAGAAAGAGACATTCAAAGAGAAGGACAC
CTACAAGCTGTTCAAGAACGGCACCCTGAAGATCAAGCACCTGAAAACCGACGACCAGGACA
TCTATAAGGTGTCCATCTACCACACCAAGGGCAAGAACGTGCTGGAAAAGATCTTCGACCTC
AAGATCCAAGAGCGGGTGTCCAAGCCTAAGATCAGCTGGACCTGCATCAACACCACACTGAC
CTGCGAAGTGATGAACGGCACAGACCCCGAGCTGAACCTGTACCAGGATGGCAAACACCTGA
AGCTGAGCCAGCGCGTGATCACCCACAAGTGGACAACAAGCCTGAGCGCCAAGTTCAAGTGC
ACCGCCGGAAACAAAGTGTCTAAAGAGTCCAGCGTCGAGCCCGTGTCTTGCCCTGAAAAAGG
ACTGGACATCTACCTGATCATCGGCATCTGTGGCGGCGGATCCCTGCTGATGGTGTTTGTGG
CTCTGCTGGTGTTCTACATCACA

[symbol]

[symbol]

[symbol]

[symbol]

[symbol]

[symbol]

[symbol]

[symbol]

[symbol]

AAATTCAGCCGCAGCGCCGATGCTCCTGCCTATAAGCAGGGACAG
AACCAGCTGTACAACGAGCTGAATCTGGGGCGCAGAGAAGAGTACGATGTGCTGGACAAGCG
GAGAGGCAGAGATCCTGAGATGGGCGGCAAGCCCAGACGGAAGAATCCTCAAGAGGGCCTGT
ATAATGAGCTGCAGAAAGACAAGATGGCCGAGGCCTACAGCGAGATCGGAATGAAGGGCGAG
CGCAGAAGAGGCAAGGGACACGATGGACTGTATCAGGGCCTGAGCACCGCCACCAAGGATAC
CTATGATGCCCTGCACATGCAGGCCCTGCCTCCAAGA
CD2 Signal peptide: SEQ ID NO: 6
CD2 Extracellular domain with D111H mutation within
the CD2 Extracellular domain: SEQ ID NO: 4
CD2 Transmembrane domain: SEQ ID NO: 10
GITR Intracellular domain: SEQ ID NO: 32
CD3ζ Intracellular domain: SEQ ID NO: 12

An exemplary “mutant CSR 10b CD2.8.DH.Gz” or “CD2.8.DH.Gz” polypeptide of the disclosure comprises or consists of the amino acid sequence of (CD8a Signal peptide, CD2 Extracellular domain with D111H mutation within the CD2 Extracellular domain, CD2 Transmembrane domain, GITR Intracellular domain, CD3ζ Intracellular domain):

(SEQ ID NO: 73)
MALPVTALLLPLALLLHAARPKEITNALETWGALGQDINLDIPSFQMSDDIDDIKWEKTSDK
KKIAQFRKEKETFKEKDTYKLFKNGTLKIKHLKTDDQDIYKVSIYHTKGKNVLEKIFDLKIQ
ERVSKPKISWTCINTTLTCEVMNGTDPELNLYQDGKHLKLSQRVITHKWTTSLSAKFKCTAG
NKVSKESSVEPVSCPEKGLDIYLIIGICGGGSLLMVFVALLVFYIT [symbol] [symbol] [symbol]
RVKFSRSADAPAYKQGQNQL
YNELNLGRREEYDVLDKRRGRDPEMGGKPRRKNPQEGLYNELQKDKMAEAYSEIGMKGERRR
GKGHDGLYQGLSTATKDTYDALHMQALPPR
CD8a Signal peptide: SEQ ID NO: 7
CD2 Extracellular domain with D111H mutation within
the CD2 Extracellular domain: SEQ
ID NO: 3
CD2 Transmembrane domain: SEQ ID NO: 9
GITR Intracellular domain: SEQ ID NO: 31
CD3ζ Intracellular domain: SEQ ID NO: 11

An exemplary polynucleotide acid sequence encoding a “mutant CSR 10b CD2.8.DH.Gz” or “CD2.8.DH.Gz” polypeptide of the disclosure comprises or consists of the nucleic acid sequence of (CD8a Signal peptide, CD2 Extracellular domain with D111H mutation within the CD2 Extracellular domain, CD2 Transmembrane domain, GITR Intracellular domain, CD3ζ Intracellular domain):

(SEQ ID NO: 74)
ATGGCTCTGCCTGTGACAGCTCTGCTGCTGCCTCTGGCTCTGCTTCTGCATGCCGCCAGACC
TAAAGAAATCACGAATGCATTGGAAACCTGGGGAGCCCTCGGCCAGGATATTAACCTGGACA
TCCCCAGCTTCCAGATGAGCGACGACATCGATGACATCAAGTGGGAGAAAACCAGCGACAAG
AAGAAGATCGCCCAGTTCCGGAAAGAGAAAGAGACATTCAAAGAGAAGGACACCTACAAGCT
GTTCAAGAACGGCACCCTGAAGATCAAGCACCTGAAAACCGACGACCAGGACATCTATAAGG
TGTCCATCTACCACACCAAGGGCAAGAACGTGCTGGAAAAGATCTTCGACCTCAAGATCCAA
GAGCGGGTGTCCAAGCCTAAGATCAGCTGGACCTGCATCAACACCACACTGACCTGCGAAGT
GATGAACGGCACAGACCCCGAGCTGAACCTGTACCAGGATGGCAAACACCTGAAGCTGAGCC
AGCGCGTGATCACCCACAAGTGGACAACAAGCCTGAGCGCCAAGTTCAAGTGCACCGCCGGA
AACAAAGTGTCTAAAGAGTCCAGCGTCGAGCCCGTGTCTTGCCCTGAAAAAGGACTGGACAT
CTACCTGATCATCGGCATCTGTGGCGGCGGATCCCTGCTGATGGTGTTTGTGGCTCTGCTGG
TGTTCTACATCACA

[symbol]

[symbol]

[symbol]

[symbol]

[symbol]

[symbol]

[symbol]

[symbol]

[symbol]

AAATTCAGCCGCAGCGCCGATGCTCCTGCCTATAAGCAGGGACAGAACCAGCTG
TACAACGAGCTGAATCTGGGGCGCAGAGAAGAGTACGATGTGCTGGACAAGCGGAGAGGCAG
AGATCCTGAGATGGGCGGCAAGCCCAGACGGAAGAATCCTCAAGAGGGCCTGTATAATGAGC
TGCAGAAAGACAAGATGGCCGAGGCCTACAGCGAGATCGGAATGAAGGGCGAGCGCAGAAGA
GGCAAGGGACACGATGGACTGTATCAGGGCCTGAGCACCGCCACCAAGGATACCTATGATGC
CCTGCACATGCAGGCCCTGCCTCCAAGA
CD8a Signal peptide: SEQ ID NO: 8
CD2 Extracellular domain with D111H mutation within
the CD2 Extracellular domain: SEQ
ID NO: 4
CD2 Transmembrane domain: SEQ ID NO: 10
GITR Intracellular domain: SEQ ID NO: 32
CD3ζ Intracellular domain: SEQ ID NO: 12

A composition of the present disclosure (e.g., a CSR) binds anti-CD2 activating agonists and anti-CD2 activating molecules but does not bind naturally occurring CD58.

Compositions comprising a CSR of the present disclosure can be incorporated into a cell delivery composition (e.g., transposon or vector) as described in detail herein, and, optionally, can be incorporated into a cell.

Cells and Modified Cells of the Disclosure

Cells and modified cells of the disclosure can be mammalian cells. Preferably, the cells and modified cells are human cells. Cells and modified cells of the disclosure can be immune cells. The immune cells of the disclosure can comprise lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts.

The immune precursor cells can comprise any cells which can differentiate into one or more types of immune cells. The immune precursor cells can comprise multipotent stem cells that can self-renew and develop into immune cells. The immune precursor cells can comprise hematopoietic stem cells (HSCs) or descendants thereof. The immune precursor cells can comprise precursor cells that can develop into immune cells. The immune precursor cells can comprise hematopoietic progenitor cells (HPCs).

Hematopoietic stem cells (HSCs) are multipotent, self-renewing cells. All differentiated blood cells from the lymphoid and myeloid lineages arise from HSCs. HSCs can be found in adult bone marrow, peripheral blood, mobilized peripheral blood, peritoneal dialysis effluent and umbilical cord blood.

HSCs can be isolated or derived from a primary or cultured stem cell. HSCs can be isolated or derived from an embryonic stem cell, a multipotent stem cell, a pluripotent stem cell, an adult stem cell, or an induced pluripotent stem cell (iPSC).

Immune precursor cells can comprise an HSC or an HSC descendent cell. Non-limiting examples of HSC descendent cells include multipotent stem cells, lymphoid progenitor cells, natural killer (NK) cells, T lymphocyte cells (T-cells), B lymphocyte cells (B-cells), myeloid progenitor cells, neutrophils, basophils, eosinophils, monocytes and macrophages.

HSCs produced by the disclosed methods can retain features of “primitive” stem cells that, while isolated or derived from an adult stem cell and while committed to a single lineage, share characteristics of embryonic stem cells. For example, the “primitive” HSCs produced by the disclosed methods retain their “stemness” following division and do not differentiate. Consequently, as an adoptive cell therapy, the “primitive” HSCs produced by the disclosed methods not only replenish their numbers, but expand in vivo. “Primitive” HSCs produced by disclosed the methods can be therapeutically-effective when administered as a single dose.

Primitive HSCs can be CD34+. Primitive HSCs can be CD34+ and CD38−. Primitive HSCs can be CD34+, CD38− and CD90+. Primitive HSCs can be CD34+, CD38−, CD90+ and CD45RA−. Primitive HSCs can be CD34+, CD38−, CD90+, CD45RA−, and CD49f+. Primitive HSCs can be CD34+, CD38−, CD90+, CD45RA−, and CD49f+.

Primitive HSCs, HSCs, and/or HSC descendent cells can be modified according to the disclosed methods to express an exogenous sequence (e.g., a chimeric antigen receptor or therapeutic protein). Modified primitive HSCs, modified HSCs, and/or modified HSC descendent cells can be forward differentiated to produce a modified immune cell including, but not limited to, a modified T cell, a modified natural killer cell and/or a modified B-cell.

The modified immune or immune precursor cells can be NK cells. The NK cells can be cytotoxic lymphocytes that differentiate from lymphoid progenitor cells. Modified NK cells can be derived from modified hematopoietic stem and progenitor cells (HSPCs) or modified HSCs. In some aspects, non-activated NK cells are derived from CD3-depleted leukapheresis (containing CD14/CD19/CD56+ cells).

The modified immune or immune precursor cells can be B cells. B cells are a type of lymphocyte that express B cell receptors on the cell surface. B cell receptors bind to specific antigens. Modified B cells can be derived from modified hematopoietic stem and progenitor cells (HSPCs) or modified HSCs.

Modified T cells of the disclosure may be derived from modified hematopoietic stem and progenitor cells (HSPCs) or modified HSCs. Unlike traditional biologics and chemotherapeutics, the disclosed modified-T cells the capacity to rapidly reproduce upon antigen recognition, thereby potentially obviating the need for repeat treatments. To achieve this, in some embodiments, modified-T cells not only drive an initial response, but also persist in the patient as a stable population of viable memory T cells to prevent potential relapses. Alternatively, in some aspects, when it is not desired, the modified-T cells do not persist in the patient.

Intensive efforts have been focused on the development of antigen receptor molecules that do not cause T cell exhaustion through antigen-independent (tonic) signaling, as well as of a modified-T cell product containing early memory T cells, especially stem cell memory (T_{SCM}) or stem cell-like T cells. Stem cell-like modified-T cells of the disclosure exhibit the greatest capacity for self-renewal and multipotent capacity to derive central memory (T_{CM}) T cells or T_{CM} like cells, effector memory (T_{EM}) and effector T cells (T_{E}), thereby producing better tumor eradication and long-term modified-T cell engraftment. A linear pathway of differentiation may be responsible for generating these cells: Naïve T cells (T_{N})>T_{SCM}>T_{CM}>T_{EM}>T_{E}>T_{TE}, whereby T_{N} is the parent precursor cell that directly gives rise to T_{SCM}, which then, in turn, directly gives rise to T_{CM}, etc. Compositions of T cells of the disclosure can comprise one or more of each parental T cell subset with T_{SCM} cells being the most abundant (e.g., T_{SCM}>T_{CM}>T_{EM}>T_{E}>T_{TE}).

The immune cell precursor can be differentiated into or is capable of differentiating into an early memory T cell, a stem cell like T-cell, a Naïve T cells (T_{N}), a T_{SCM}, a T_{CM}, a T_{EM}, a T_{E}, or a T_{TE}. The immune cell precursor can be a primitive HSC, an HSC, or a HSC descendent cell of the disclosure. The immune cell can be an early memory T cell, a stem cell like T-cell, a Naïve T cells (T_{N}), a T_{SCM}, a T_{CM}, a T_{EM}, a T_{E}, or a T_{TE}.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of a plurality of modified T cells in the population expresses one or more cell-surface marker(s) of an early memory T cell. The population of modified early memory T cells comprises a plurality of modified stem cell-like T cells. The population of modified early memory T cells comprises a plurality of modified T_{SCM} cells. The population of modified early memory T cells comprises a plurality of modified T_{CM} cells.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a stem cell-like T cell. The population of modified stem cell-like T cells comprises a plurality of modified T_{SCM} cells. The population of modified stem cell-like T cells comprises a plurality of modified T_{CM} cells.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2RP. The cell-surface markers can comprise one or more of CD45RA, CD95, IL-2RP, CCR7, and CD62L.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the plurality of modified T cells in the population expresses one or more cell-surface marker(s) of a central memory T cell (T_{CM}) or a T_{CM}-like cell; and wherein the one or more cell-surface marker(s) comprise CD45RO and CD62L. The cell-surface markers can comprise one or more of CD45RO, CD95, IL-2Rβ, CCR7, and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a naïve T cell (T_{N}). The cell-surface markers can comprise one or more of CD45RA, CCR7 and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of an effector T-cell (modified T_{EFF}). The cell-surface markers can comprise one or more of CD45RA, CD95, and IL-2Rβ.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells of the population expresses one or more cell-surface marker(s) of a stem cell-like T cell, a stem memory T cell (T_{SCM}) or a central memory T cell (T_{CM}).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 or wherein at least about 70% to about 99%, about 75% to about 95% or about 85% to about 95% of the population of modified cells express one or more cell-surface marker(s) comprising CD34 (e.g., comprise the cell-surface marker phenotype CD34+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and do not express one or more cell-surface marker(s) comprising CD38, or wherein at least about 45% to about 90%, about 50% to about 80% or about 65% to about 75% of the population of modified cells express one or more cell-surface marker(s) comprising CD34 and do not express one or more cell-surface marker(s) comprising CD38 (e.g., comprise the cell-surface marker phenotype CD34+ and CD38-).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38, or wherein at least about 0.2% to about 40%, about 0.2% to about 30%, about 0.2% to about 2% or 0.5% to about 1.5% of the population of modified cells express one or more cell-surface marker(s) comprising CD34 and CD90 and do not express one or more cell-surface marker(s) comprising CD38 (e.g., comprise the cell-surface marker phenotype CD34+, CD38− and CD90+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38 and CD45RA, or wherein at least about 0.2% to about 40%, about 0.2% to about 30%, about 0.2% to about 2% or 0.5% to about 1.5% of the population of modified cells express one or more cell-surface marker(s) comprising CD34 and CD90 and do not express one or more cell-surface marker(s) comprising CD38 and CD45RA (e.g., comprise the cell-surface marker phenotype CD34+, CD38−, CD90+, CD45RA−).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34, CD90 and CD49f and do not express one or more cell-surface marker(s) comprising CD38 and CD45RA, or wherein at least about 0.02% to about 30%, about 0.02% to about 2%, about 0.04% to about 2% or about 0.04% to about 1% of the population of modified cells express one or more cell-surface marker(s) comprising CD34, CD90 and CD49f and do not express one or more cell-surface marker(s) comprising CD38 and CD45RA (e.g., comprise the cell-surface marker phenotype CD34+, CD38-, CD90+, CD45RA- and CD49f+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD45RA, or wherein at least about 0.2% to about 5%, about 0.2% to about 3% or about 0.4% to about 3% of the population of modified cells express one or more cell-surface marker(s) comprising CD34 and CD90 and do not express one or more cell-surface marker(s) comprising CD45RA (e.g., comprise the cell-surface marker phenotype CD34+, CD90+ and CD45RA−).

Compositions and methods of producing and/or expanding the immune cells or immune precursor cells (e.g., the disclosed modified T-cells) and buffers for maintaining or enhancing a level of cell viability and/or a stem-like phenotype of the immune cells or immune precursor cells (e.g., the disclosed modified T-cells) are disclosed elsewhere herein and are disclosed in more detail in U.S. Pat. No. 10,329,543 and PCT Publication No. WO 2019/173636.

Cells and modified cells of the disclosure can be somatic cells. Cells and modified cells of the disclosure can be differentiated cells. Cells and modified cells of the disclosure can be autologous cells or allogenic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

Methods of Expressing a Chimeric Antigen Receptor

The disclosure provides methods of expressing a CAR on the surface of a cell. The method comprises (a) obtaining a cell population; (b) contacting the cell population to a composition comprising a CAR or a sequence encoding the CAR, under conditions sufficient to transfer the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface.

In some aspects, the cell population can comprise leukocytes and/or CD4+ and CD8+ leukocytes. The cell population can comprise CD4+ and CD8+ leukocytes in an optimized ratio. The optimized ratio of CD4+ to CD8+ leukocytes does not naturally occur in vivo. The cell population can comprise a tumor cell.

In some aspects, the conditions sufficient to transfer the CAR or the sequence encoding the CAR, transposon, or vector across a cell membrane of at least one cell in the cell population comprises at least one of an application of one or more pulses of electricity at a specified voltage, a buffer, and one or more supplemental factor(s). In some aspects, the conditions suitable for integration of the sequence encoding the CAR comprise at least one of a buffer and one or more supplemental factor(s).

The buffer can comprise PBS, HBSS, OptiMEM, BTXpress, Amaxa Nucleofector, Human T cell nucleofection buffer or any combination thereof. The one or more supplemental factor(s) can comprise (a) a recombinant human cytokine, a chemokine, an interleukin or any combination thereof; (b) a salt, a mineral, a metabolite or any combination thereof; (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof can comprise IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L or any combination thereof. The salt, the mineral, the metabolite or any combination thereof can comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, antibiotics, pH adjusters, Earle's Salts, 2-Mercaptoethanol, Human transferrin, Recombinant human insulin, Human serum albumin, Nucleofector PLUS Supplement, KCL, MgCl₂, Na₂HPO₄, NAH₂PO₄, Sodium lactobionate, Mannitol, Sodium succinate, Sodium Chloride, CINa, Glucose, Ca(NO₃)₂, Tris/HCl, K₂HPO₄, KH₂PO₄, Polyethylenimine, Poly-ethylene-glycol, Poloxamer 188, Poloxamer 181, Poloxamer 407, Poly-vinylpyrrolidone, Pop313, Crown-5, or any combination thereof. The cell medium can comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof. The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3β) (e.g. TWS119), or any combination thereof. Examples of such inhibitors can include Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

The expansion and selection steps can occur concurrently or sequentially. The expansion can occur prior to selection. The expansion can occur following selection, and, optionally, a further (i.e. second) selection can occur following expansion. Concurrent expansion and selection can be simultaneous. The expansion and/or selection steps can proceed for a period of 10 to 14 days, inclusive of the endpoints.

The expansion can comprise contacting at least one cell of the modified cell population with an antigen to stimulate the at least one cell through the CAR, thereby generating an expanded cell population. The antigen can be presented on the surface of a substrate. The substrate can have any form, including, but not limited to a surface, a well, a bead or a plurality thereof, and a matrix. The substrate can further comprise a paramagetic or magnetic component. The antigen can be presented on the surface of a substrate, wherein the substrate is a magnetic bead, and wherein a magnet can be used to remove or separate the magnetic beads from the modified and expanded cell population. The antigen can be presented on the surface of a cell or an artificial antigen presenting cell. Artificial antigen presenting cells can include, but are not limited to, tumor cells and stem cells.

In some aspects wherein the transposon or vector comprises a selection gene, the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

The disclosure provides a composition comprising the modified, expanded and selected cell population of the methods described herein.

A more detailed description of methods for expressing a CAR on the surface of a cell is disclosed in PCT Publication No. WO 2019/049816 and PCT/US2019/049816.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The composition can modify gene expression by decreasing gene expression. The composition can modify gene expression by transiently modifying gene expression (e.g., for the duration of binding of the ligand to the exogenous receptor). The composition can modify gene expression acutely (e.g., the ligand reversibly binds to the exogenous receptor). The composition can modify gene expression chronically (e.g., the ligand irreversibly binds to the exogenous receptor).

The exogenous receptor can comprise an endogenous receptor with respect to the genomic sequence of the cell. Exemplary receptors include, but are not limited to, intracellular receptors, cell-surface receptors, transmembrane receptors, ligand-gated ion channels, and G-protein coupled receptors.

The exogenous receptor can comprise a non-naturally occurring receptor. The non-naturally occurring receptor can be a synthetic, modified, recombinant, mutant or chimeric receptor. The non-naturally occurring receptor can comprise one or more sequences isolated or derived from a T-cell receptor (TCR). The non-naturally occurring receptor can comprise one or more sequences isolated or derived from a scaffold protein. In some aspects, including those wherein the non-naturally occurring receptor does not comprise a transmembrane domain, the non-naturally occurring receptor interacts with a second transmembrane, membrane-bound and/or an intracellular receptor that, following contact with the non-naturally occurring receptor, transduces an intracellular signal. The non-naturally occurring receptor can comprise a transmembrane domain. The non-naturally occurring receptor can interact with an intracellular receptor that transduces an intracellular signal. The non-naturally occurring receptor can comprise an intracellular signaling domain. The non-naturally occurring receptor can be a chimeric ligand receptor (CLR). The CLR can be a chimeric antigen receptor (CAR).

The sequence encoding the inducible promoter of comprises a sequence encoding an NFκB promoter, a sequence encoding an interferon (IFN) promoter or a sequence encoding an interleukin-2 promoter. In some aspects, the IFN promoter is an IFNγ promoter. The inducible promoter can be isolated or derived from the promoter of a cytokine or a chemokine. The cytokine or chemokine can comprise IL2, IL3, IL4, IL5, IL6, IL10, IL12, IL13, IL17A/F, IL21, IL22, IL23, transforming growth factor beta (TGFβ), colony stimulating factor 2 (GM-CSF), interferon gamma (IFNγ), Tumor necrosis factor alpha (TNFα), LTα, perforin, Granzyme C (Gzmc), Granzyme B (Gzmb), C-C motif chemokine ligand 5 (CCL5), C-C motif chemokine ligand 4 (Ccl4), C-C motif chemokine ligand 3 (Ccl3), X-C motif chemokine ligand 1 (Xcl1) or LIF interleukin 6 family cytokine (Lif).

The inducible promoter can be isolated or derived from the promoter of a gene comprising a surface protein involved in cell differentiation, activation, exhaustion and function. In some aspects, the gene comprises CD69, CD71, CTLA4, PD-1, TIGIT, LAG3, TIM-3, GITR, MHCII, COX-2, FASL or 4-1BB.

The inducible promoter can be isolated or derived from the promoter of a gene involved in CD metabolism and differentiation. The inducible promoter can be isolated or derived from the promoter of Nr4a1, Nr4a3, Tnfrsf9 (4-1BB), Sema7a, Zfp36l2, Gadd45b, Dusp5, Dusp6 and Neto2.

In some aspects, the inducible transgene construct comprises or drives expression of a signaling component downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of which are disclosed in PCT Publication No. WO 2019/173636 and PCT Application No. PCT/US2019/049816.

Armored Cells

The modified cells of disclosure (e.g., CAR T-cells) can be further modified to enhance their therapeutic potential. Alternatively, or in addition, the modified cells may be further modified to render them less sensitive to immunologic and/or metabolic checkpoints. Modifications of this type “armor” the cells, which, following the modification, may be referred to here as “armored” cells (e.g., armored T-cells). Armored cells may be produced by, for example, blocking and/or diluting specific checkpoint signals delivered to the cells (e.g., checkpoint inhibition) naturally, within the tumor immunosuppressive microenvironment.

An armored cell of the disclosure can be derived from any cell, for example, a T cell, a NK cell, a hematopoietic progenitor cell, a peripheral blood (PB) derived T cell (including a T cell isolated or derived from G-CSF-mobilized peripheral blood), or an umbilical cord blood (UCB) derived T cell. An armored cell (e.g., armored T-cell) can comprise one or more of a chimeric ligand receptor (CLR comprising a protein scaffold, an antibody, an ScFv, or an antibody mimetic)/chimeric antigen receptor (CAR comprising a protein scaffold, an antibody, an ScFv, or an antibody mimetic), a CARTyrin (a CAR comprising a Centyrin), and/or a VCAR (a CAR comprising a camelid VHH or a single domain VH). An armored cell (e.g., armored T-cell) can comprise an inducible proapoptotic polypeptide as disclosed herein. An armored cell (e.g., armored T-cell) can comprise an exogenous sequence. The exogenous sequence can comprise a sequence encoding a therapeutic protein. Exemplary therapeutic proteins may be nuclear, cytoplasmic, intracellular, transmembrane, cell-surface bound, or secreted proteins. Exemplary therapeutic proteins expressed by the armored cell (e.g., armored T-cell) may modify an activity of the armored cell or may modify an activity of a second cell. An armored cell (e.g., armored T-cell) can comprise a selection gene or a selection marker. An armored cell (e.g., armored T-cell) can comprise a synthetic gene expression cassette (also referred to herein as an inducible transgene construct).

The modified cells of disclosure (e.g., CAR T-cells) can be further modified to silence or reduce expression one or more gene(s) encoding receptor(s) of inhibitory checkpoint signals to produce an armored cell (e.g., armored CAR T-cell). Receptors of inhibitory checkpoint signals are expressed on the cell surface or within the cytoplasm of a cell. Silencing or reducing expressing of the gene encoding the receptor of the inhibitory checkpoint signal results a loss of protein expression of the inhibitory checkpoint receptors on the surface or within the cytoplasm of an armored cell. Thus, armored cells having silenced or reduced expression of one or more genes encoding an inhibitory checkpoint receptor is resistant, non-receptive or insensitive to checkpoint signals. The resistance or decreased sensitivity of the armored cell to inhibitory checkpoint signals enhances the therapeutic potential of the armored cell in the presence of these inhibitory checkpoint signals. Non-limiting examples of inhibitory checkpoint signals (and proteins that induce immunosuppression) are disclosed in PCT Publication No. WO 2019/173636. Preferred examples of inhibitory checkpoint signals that may be silenced include, but are not limited to, PD-I and TGFβRII.

The modified cells of disclosure (e.g., CAR T-cells) can be further modified to silence or reduce expression of one or more gene(s) encoding intracellular proteins involved in checkpoint signaling to produce an armored cell (e.g., armored CAR T-cell). The activity of the modified cells may be enhanced by targeting any intracellular signaling protein involved in a checkpoint signaling pathway, thereby achieving checkpoint inhibition or interference to one or more checkpoint pathways. Non-limiting examples of intracellular signaling proteins involved in checkpoint signaling are disclosed in PCT Publication No. WO 2019/173636.

The modified cells of disclosure (e.g., CAR T-cells) can be further modified to silence or reduce expression of one or more gene(s) encoding a transcription factor that hinders the efficacy of a therapy to produce an armored cell (e.g., armored CAR T-cell). The activity of modified cells may be enhanced or modulated by silencing or reducing expression (or repressing a function) of a transcription factor that hinders the efficacy of a therapy. Non-limiting examples of transcription factors that may be modified to silence or reduce expression or to repress a function thereof include, but are not limited to, the exemplary transcription factors are disclosed in PCT Publication No. WO 2019/173636.

The modified cells of disclosure (e.g., CAR T-cells) can be further modified to silence or reduce expression of one or more gene(s) encoding a cell death or cell apoptosis receptor to produce an armored cell (e.g., armored CAR T-cell). Interaction of a death receptor and its endogenous ligand results in the initiation of apoptosis. Disruption of an expression, an activity, or an interaction of a cell death and/or cell apoptosis receptor and/or ligand render a modified cell less receptive to death signals, consequently, making the armored cell more efficacious in a tumor environment. Non-limiting examples of cell death and/or cell apoptosis receptors and ligands are disclosed in PCT Publication No. WO 2019/173636. A preferred example of cell death receptor which may be modified is Fas (CD95).

The modified cells of disclosure (e.g., CAR T-cells) can be further modified to silence or reduce expression of one or more gene(s) encoding a metabolic sensing protein to produce an armored cell (e.g., armored CAR T-cell). Disruption to the metabolic sensing of the immunosuppressive tumor microenvironment (characterized by low levels of oxygen, pH, glucose and other molecules) by a modified cell leads to extended retention of T-cell function and, consequently, more tumor cells killed per cell. Non-limiting examples of metabolic sensing genes and proteins are disclosed in PCT Publication No. WO 2019/173636. A preferred example, HIF1a and VHL play a role in T-cell function while in a hypoxic environment. An armored T-cell may have silenced or reduced expression of one or more genes encoding HIF1a or VHL.

The modified cells of disclosure (e.g., CAR T-cells) can be further modified to silence or reduce expression of one or more gene(s) encoding proteins that that confer sensitivity to a cancer therapy, including a monoclonal antibody, to produce an armored cell (e.g., armored CAR T-cell). Thus, an armored cell can function and may demonstrate superior function or efficacy whilst in the presence of a cancer therapy (e.g., a chemotherapy, a monoclonal antibody therapy, or another anti-tumor treatment). Non-limiting examples of proteins involved in conferring sensitivity to a cancer therapy are disclosed in PCT Publication No. WO 2019/173636.

The modified cells of disclosure (e.g., CAR T-cells) can be further modified to silence or reduce expression of one or more gene(s) encoding a growth advantage factor to produce an armored cell (e.g., armored CAR T-cell). Silencing or reducing expression of an oncogene can confer a growth advantage for the cell. For example, silencing or reducing expression (e.g., disrupting expression) of a TET2 gene during a CAR T-cell manufacturing process results in the generation of an armored CAR T-cell with a significant capacity for expansion and subsequent eradication of a tumor when compared to a non-armored CAR T-cell lacking this capacity for expansion. This strategy may be coupled to a safety switch (e.g., an iC9 safety switch described herein), which permits the targeted disruption of an armored CAR T-cell in the event of an adverse reaction from a subject or uncontrolled growth of the armored CAR T-cell. Non-limiting examples of growth advantage factors are disclosed in PCT Publication No. WO 2019/173636.

The modified cells of disclosure (e.g., CAR T-cells) can be further modified to express a modified/chimeric checkpoint receptor to produce an armored T-cell of the disclosure.

The modified/chimeric checkpoint receptor can comprise a null receptor, decoy receptor or dominant negative receptor. A null receptor, decoy receptor or dominant negative receptor can be modified/chimeric receptor/protein. A null receptor, decoy receptor or dominant negative receptor can be truncated for expression of the intracellular signaling domain. Alternatively, or in addition, a null receptor, decoy receptor or dominant negative receptor can be mutated within an intracellular signaling domain at one or more amino acid positions that are determinative or required for effective signaling. Truncation or mutation of null receptor, decoy receptor or dominant negative receptor can result in loss of the receptor's capacity to convey or transduce a checkpoint signal to the cell or within the cell.

For example, a dilution or a blockage of an immunosuppressive checkpoint signal from a PD-L1 receptor expressed on the surface of a tumor cell may be achieved by expressing a modified/chimeric PD-1 null receptor on the surface of an armored cell (e.g., armored CAR T-cell), which effectively competes with the endogenous (non-modified) PD-1 receptors also expressed on the surface of the armored cell to reduce or inhibit the transduction of the immunosuppressive checkpoint signal through endogenous PD-I receptors of the armored cell. In this non-limiting example, competition between the two different receptors for binding to PD-L1 expressed on the tumor cell reduces or diminishes a level of effective checkpoint signaling, thereby enhancing a therapeutic potential of the armored cell expressing the PD-1 null receptor.

The modified/chimeric checkpoint receptor can comprise a null receptor, decoy receptor or dominant negative receptor that is a transmembrane receptor, a membrane-associated or membrane-linked receptor/protein or an intracellular receptor/protein. Exemplary null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636.

The modified/chimeric checkpoint receptor can comprise a switch receptor. Exemplary switch receptors comprise a modified/chimeric receptor/protein wherein a native or wild type intracellular signaling domain is switched or replaced with a different intracellular signaling domain that is either non-native to the protein and/or not a wild-type domain. For example, replacement of an inhibitory signaling domain with a stimulatory signaling domain would switch an immunosuppressive signal into an immunostimulatory signal. Alternatively, replacement of an inhibitory signaling domain with a different inhibitory domain can reduce or enhance the level of inhibitory signaling. Expression or overexpression, of a switch receptor can result in the dilution and/or blockage of a cognate checkpoint signal via competition with an endogenous wild-type checkpoint receptor (not a switch receptor) for binding to the cognate checkpoint receptor expressed within the immunosuppressive tumor microenvironment. Armored cells (e.g., armored CAR T-cells) can comprise a sequence encoding a switch receptor, leading to the expression of one or more switch receptors, and consequently, altering an activity of an armored cell. Armored cells (e.g., armored CAR T-cells) can express a switch receptor that targets an intracellularly expressed protein downstream of a checkpoint receptor, a transcription factor, a cytokine receptor, a death receptor, a metabolic sensing molecule, a cancer therapy, an oncogene, and/or a tumor suppressor protein or gene.

Exemplary switch receptors can comprise or can be derived from a protein including, but are not limited to, the signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene.

The modified cells of disclosure (e.g., CAR T-cells) can be further modified to express a CLR/CAR that mediates conditional gene expression to produce an armored T-cell. The combination of the CLR/CAR and the condition gene expression system in the nucleus of the armored T-cell constitutes a synthetic gene expression system that is conditionally activated upon binding of cognate ligand(s) with CLR or cognate antigen(s) with CAR. This system may help to ‘armor’ or enhance therapeutic potential of modified T-cells by reducing or limiting synthetic gene expression at the site of ligand or antigen binding, at or within the tumor environment for example.

Gene Editing Compositions and Methods

A modified cell be produced by introducing a transgene into the cell. The introducing step may comprise delivery of a nucleic acid sequence, a transgene, and/or a genomic editing construct via a non-transposition delivery system.

Introducing a nucleic acid sequence, transgene and/or a genomic editing construct into a cell ex vivo, in vivo, in vitro or in situ can comprise one or more of topical delivery, adsorption, absorption, electroporation, spin-fection, co-culture, transfection, mechanical delivery, sonic delivery, vibrational delivery, magnetofection or by nanoparticle-mediated delivery. Introducing a nucleic acid sequence, a transgene and/or a genomic editing construct into a cell ex vivo, in vivo, in vitro or in situ can comprise liposomal transfection, calcium phosphate transfection, fugene transfection, and dendrimer-mediated transfection. Introducing a nucleic acid sequence, a transgene, and/or a genomic editing construct into a cell ex vivo, in vivo, in vitro or in situ by mechanical transfection can comprise cell squeezing, cell bombardment, or gene gun techniques. Introducing a nucleic acid sequence, transgene and/or a genomic editing construct into a cell ex vivo, in vivo, in vitro or in situ by nanoparticle-mediated transfection can comprise liposomal delivery, delivery by micelles, and delivery by polymerosomes.

Introducing a nucleic acid sequence, transgene and/or a genomic editing construct into a cell ex vivo, in vivo, in vitro or in situ can comprise a non-viral vector. The non-viral vector can comprise a nucleic acid. The non-viral vector can comprise plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). The non-viral vector can comprise a transposon as described herein.

Introducing a nucleic acid sequence, transgene and/or a genomic editing construct into a cell ex vivo, in vivo, in vitro or in situ can comprise a viral vector. The viral vector can be a non-integrating non-chromosomal vector. Non-limiting examples of non-integrating non-chromosomal vectors include adeno-associated virus (AAV), adenovirus, and herpes viruses. The viral vector can be an integrating chromosomal vector. Non-limiting examples of integrating chromosomal vectors include adeno-associated vectors (AAV), Lentiviruses, and gamma-retroviruses.

Introducing a nucleic acid sequence, transgene and/or a genomic editing construct into a cell ex vivo, in vivo, in vitro or in situ can comprise a combination of vectors. Non-limiting examples of vector combinations include viral and non-viral vectors, a plurality of non-viral vectors, or a plurality of viral vectors. Non-limiting examples of vector combinations include a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

Genome modification can comprise introducing a nucleic acid sequence, transgene and/or a genomic editing construct into a cell ex vivo, in vivo, in vitro or in situ to stably integrate a nucleic acid sequence. The stable chromosomal integration can be a random integration, a site-specific integration, or a biased integration. The site-specific integration can be non-assisted or assisted. The assisted site-specific integration is co-delivered with a site-directed nuclease. The site-directed nuclease comprises a transgene with 5′ and 3′ nucleotide sequence extensions that contain a percentage homology to upstream and downstream regions of the site of genomic integration. The transgene with homologous nucleotide extensions enable genomic integration by homologous recombination, microhomology-mediated end joining, or nonhomologous end-joining. The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PDI, any immunosuppressive gene, and genes involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

Enzymes can be used to create strand breaks in the host genome to facilitate delivery or integration of the transgene. Enzymes can create single-strand breaks or double-strand breaks. Non-limiting examples of break-inducing enzymes include transposases, integrases, endonucleases, CRISPR-Cas9, transcription activator-like effector nucleases (TALEN), zinc finger nucleases (ZFN), Cas-CLOVER™, and CPF1. Break-inducing enzymes can be delivered to the cell encoded in DNA, encoded in mRNA, as a protein, or as a nucleoprotein complex with a guide RNA (gRNA).

The site-specific transgene integration can be controlled by a vector-mediated integration site bias. Vector-mediated integration site bias can controlled by the chosen lentiviral vector or by the chosen gamma-retroviral vector.

The site-specific transgene integration site can be a non-stable chromosomal insertion. The integrated transgene can be become silenced, removed, excised, or further modified. The genome modification can be a non-stable integration of a transgene. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In one aspect, the transient non-chromosomal insertion of a transgene does not integrate into a chromosome and the modified genetic material is not replicated during cell division.

The genome modification can be a semi-stable or persistent non-chromosomal integration of a transgene. A DNA vector encodes a Scaffold/matrix attachment region (S-MAR) module that binds to nuclear matrix proteins for episomal retention of a non-viral vector allowing for autonomous replication in the nucleus of dividing cells.

The genome modification can be a non-stable chromosomal integration of a transgene. The integrated transgene can become silenced, removed, excised, or further modified.

The modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. The modification to the genome by transgene insertion can occur via CRISPR-Cas9, TALEN, ZFNs, Cas-CLOVER™, and cpfl.

In gene editing systems that involve inserting new or existing nucleotides/nucleic acids, insertion tools (e.g., DNA template vectors, transposable elements (transposons or retrotransposons) must be delivered to the cell in addition to the cutting enzyme (e.g., a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. An example recombinase that may be used as a cutting enzyme is the CRE recombinase. Non-limiting examples of integrases that may be used in insertion tools include viral based enzymes taken from any of a number of viruses including AAV, gamma retrovirus, and lentivirus. Examples transposons/retrotransposons that may be used in insertion tools are described in more detail herein.

A cell with an ex vivo, in vivo, in vitro or in situ genomic modification can be a germline cell or a somatic cell. The modified cell can be a human, non-human, mammalian, rat, mouse, or dog cell. The modified cell can be differentiated, undifferentiated, or immortalized. The modified undifferentiated cell can be a stem cell. The modified undifferentiated cell can be an induced pluripotent stem cell. The modified cell can be an immune cell. The modified cell can be a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, from whole blood, from leukapheresis, or from an immortalized cell line. A detailed description for isolating cells from a leukapheresis product or blood is disclosed in in PCT Publication No. WO 2019/173636 and PCT/US2019/049816.

The present disclosure provides a gene editing composition and/or a cell comprising the gene editing composition. The gene editing composition can comprise a sequence encoding a DNA binding domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. The gene editing composition can further comprise a guide sequence. The guide sequence comprises an RNA sequence.

The disclosure provides compositions comprising a small, Cas9 (Cas9) operatively-linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease. A Staphylococcus aureus Cas9 with an active catalytic site comprises the amino acid sequence of SEQ ID NO: 79.

The disclosure provides compositions comprising an inactivated, small, Cas9 (dSaCas9) operatively-linked to an effector. The disclosure provides a fusion protein comprising, consisting essentially of or consisting of a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease. A dSaCas9 comprises the amino acid sequence of SEQ ID NO: 80, which includes a D10A and a N580A mutation to inactivate the catalytic site.

The disclosure provides compositions comprising an inactivated Cas9 (dCas9) operatively-linked to an effector. The disclosure provides a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptoccocus pyogenes. The dCas9 can comprise a dCas9 with substitutions at amino acid positions 10 and 840, which inactivate the catalytic site. In some aspects, these substitutions are D10A and H840A. The dCas9 can comprise the amino acid sequence of SEQ ID NO: 81 or SEQ ID NO: 82.

An exemplary Clo051 nuclease domain comprises, consists essentially of or consists of, the amino acid sequence of SEQ ID NO: 83.

An exemplary dCas9-Clo051 (Cas-CLOVER) fusion protein can comprise, consist essentially of, or consist of, the amino acid sequence of SEQ ID NO: 84. The exemplary dCas9-Clo051 fusion protein can be encoded by a polynucleotide which comprises, consists essentially of, or consists of, the nucleic acid sequence of SEQ ID NO: 85. The nucleic acid encoding the dCas9-Clo051 fusion protein can be DNA or RNA.

An exemplary dCas9-Clo051 (Cas-CLOVER) fusion protein can comprise, consist essentially of, or consist of, the amino acid sequence of SEQ ID NO: 86. The exemplary dCas9-Clo051 fusion protein can be encoded by a polynucleotide which comprises, consists essentially of, or consists of, the nucleic acid sequence of SEQ ID NO: 87. The nucleic acid encoding the dCas9-Clo051 fusion protein can be DNA or RNA.

A cell comprising the gene editing composition can express the gene editing composition stably or transiently. Preferably, the gene editing composition is expressed transiently. The guide RNA can comprise a sequence complementary to a target sequence within a genomic DNA sequence. The target sequence within a genomic DNA sequence can be a target sequence within a safe harbor site of a genomic DNA sequence.

Gene editing compositions, including Cas-CLOVER, and methods of using these compositions for gene editing are described in detail in U.S. Patent Publication Nos. 2017/0107541, 2017/0114149, 2018/0187185 and U.S. Pat. No. 10,415,024.

Gene editing tools can also be delivered to cells using one or more poly(histidine)-based micelles. Poly(histidine) (e.g., poly(L-histidine)), is a pH-sensitive polymer due to the imidazole ring providing an electron lone pair on the unsaturated nitrogen. That is, poly(histidine) has amphoteric properties through protonation-deprotonation. In particular, at certain pHs, poly(histidine)-containing triblock copolymers may assemble into a micelle with positively charged poly(histidine) units on the surface, thereby enabling complexing with the negatively-charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA is enabled by delivery of a nuclease using the poly(histidine)-based micelles. Without wishing to be bound by a particular theory, it is believed that believed that in the micelles that are formed by the various triblock copolymers, the hydrophobic blocks aggregate to form a core, leaving the hydrophilic blocks and poly(histidine) blocks on the ends to form one or more surrounding layer.

In an aspect, the disclosure provides triblock copolymers made of a hydrophilic block, a hydrophobic block, and a charged block. In some aspects, the hydrophilic block may be poly(ethylene oxide) (PEO), and the charged block may be poly(L-histidine). An example triblock copolymer that can be used is a PEO-b-PLA-b-PHIS, with variable numbers of repeating units in each block varying by design.

Diblock copolymers that can be used as intermediates for making triblock copolymers can have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(F-caprolactone) (PCL), and poly (trimethylene carbonate) (PTMC). Polymeric micelles comprised of 100% PEGylated surfaces possess improved in vitro chemical stability, augmented in vivo bioavailablity, and prolonged blood circulatory half-lives.

Polymeric vesicles, polymersomes and poly(Histidine)-based micelles, including those that comprise triblock copolymers, and methods of making the same, are described in further detail in U.S. Pat. Nos. 7,217,427; 7,868,512; 6,835,394; 8,808,748; 10,456,452; U.S. Publication Nos. 2014/0363496; 2017/0000743; and 2019/0255191; and PCT Publication No. WO 2019/126589.

Transposon and Vector Compositions

The present disclosure provides compositions and methods for delivering an antibody (e.g., scFv) or a CAR (e.g., comprising an scFv) to a cell or a population of cells. Non-limiting examples of compositions for delivery of a composition of the disclosure to a cell or a population of cells include a transposon or a vector. Thus, the present disclosure provides a transposon comprising an antibody (e.g., scFv) or a CAR (e.g., comprising an scFv) or a vector comprising an antibody (e.g., scFv) or a CAR (e.g., comprising an scFv).

A transposon comprising a CAR of the disclosure or a vector comprising a CAR of the disclosure can further comprise a sequence encoding an inducible proapoptotic polypeptide. Alternatively, or in addition, one transposon or one vector can comprise a CAR of the disclosure and a second transposon or second vector can comprise a sequence encoding an inducible proapoptotic polypeptide of the disclosure. Inducible proapoptotic polypeptides are described in more detail herein.

A transposon comprising a CAR of the disclosure or a vector comprising a CAR of the disclosure can further comprise a sequence encoding a chimeric stimulatory receptor (CSR). Alternatively, or in addition, one transposon or one vector can comprise a CAR of the disclosure and a second transposon or a second vector can comprise a sequence encoding a CSR of the disclosure. Chimeric stimulatory receptors are described in more detail herein.

A transposon comprising a CAR of the disclosure or a vector comprising a CAR of the disclosure can further comprise a sequence encoding a recombinant HLA-E polypeptide. Alternatively, or in addition, one transposon or one vector can comprise a CAR of the disclosure and a second transposon or a second vector can comprise a sequence encoding a recombinant HLA-E polypeptide. Recombinant HLA-E polypeptide are described in more detail herein.

A transposon comprising a CAR of the disclosure or a vector comprising a CAR of the disclosure can further comprise a selection gene. The selection gene can encode a gene product essential for cell viability and survival. The selection gene can encode a gene product essential for cell viability and survival when challenged by selective cell culture conditions. Selective cell culture conditions may comprise a compound harmful to cell viability or survival and wherein the gene product confers resistance to the compound. Non-limiting examples of selection genes include neo (conferring resistance to neomycin), DHFR (encoding Dihydrofolate Reductase and conferring resistance to Methotrexate), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCF, RAD51C (encoding RAD51 Paralog C), GCS (encoding glucosylceramide synthase), NKX2.2 (encoding NK2 Homeobox 2), or any combination thereof.

In a preferred aspect, the selection gene encodes a DHFR mutein enzyme. The DHFR mutein enzyme comprises, consists essentially of, or consists of the amino acid sequence of SEQ ID NO: 88. The DHFR mutein enzyme is encoded by a polynucleotide comprising, consisting essential of, or consisting of the nucleic acid sequence of SEQ ID NO: 89. The amino acid sequence of the DHFR mutein enzyme can further comprise a mutation at one or more of positions 80, 113, or 153. The amino acid sequence of the DHFR mutein enzyme can comprise one or more of a substitution of a Phenylalanine (F) or a Leucine (L) at position 80, a substitution of a Leucine (L) or a Valine (V) at position 113, and a substitution of a Valine (V) or an Aspartic Acid (D) at position 153.

A transposon comprising a CAR of the disclosure or a vector comprising a CAR of the disclosure can further comprise at least one self-cleaving peptide. For example, a self-cleaving peptide can be located between a CAR (e.g., comprising an scFv) and an inducible proapoptotic polypeptide; or, a self-cleaving peptide can be located between a CAR (e.g., comprising an scFv) and protein encoded by a selection gene.

A transposon comprising a CAR of the disclosure or a vector comprising a CAR of the disclosure can further comprise at least two self-cleaving peptides. For example, a first self-cleaving peptide is located upstream or immediately upstream of a CAR and a second self-cleaving peptide is located downstream or immediately downstream of a CAR; or, the first self-cleaving peptide and the second self-cleaving peptide flank a CAR. For example, a first self-cleaving peptide is located upstream or immediately upstream of an inducible proapoptotic polypeptide and a second self-cleaving peptide is located downstream or immediately downstream of an inducible proapoptotic polypeptide; or, the first self-cleaving peptide and the second self-cleaving peptide flank an inducible proapoptotic polypeptide. For example, a first self-cleaving peptide is located upstream or immediately upstream of protein encoded by a selection gene and a second self-cleaving peptide is located downstream or immediately downstream of a protein encoded by a selection gene; or, the first self-cleaving peptide and the second self-cleaving peptide flank a protein encoded by a selection gene.

Non-limiting examples of self-cleaving peptides include a T2A peptide, GSG-T2A peptide, an E2A peptide, a GSG-E2A peptide, an F2A peptide, a GSG-F2A peptide, a P2A peptide, or a GSG-P2A peptide. A T2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 90. A GSG-T2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 91. A GSG-T2A polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 92. A E2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 93. A GSG-E2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 94. A F2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 95. A GSG-F2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 96. A P2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 97. A GSG-P2A peptide comprises, consists essential of, or consists of, the amino acid sequence at least 75%, 80%, 85%, 90%, 95%, 96%, 97%, 98%, 99% or 100% (or any percentage in between) identical to SEQ ID NO: 98.

Transposition Systems

The present disclosure provides a transposon comprising a protein scaffold as disclosed herein or the present disclosure provides a transposon comprising an antibody (e.g., scFv) or a CAR (e.g., comprising an scFv) as disclosed herein. In a preferred aspect, the transposon is a plasmid DNA transposon comprising a nucleotide sequence encoding a scFv or CAR (e.g., comprising an scFv) as disclosed herein flanked by two cis-regulatory insulator elements. The present disclosure also provides a composition comprising a transposon. In a preferred aspect, the composition comprising the transposon further comprises a plasmid comprising a nucleotide sequence encoding a transposase. The nucleotide sequence encoding the transposase may be a DNA sequence or an RNA sequence. Preferably, the sequence encoding the transposase is an mRNA sequence.

A transposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB) transposase. The sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequence for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 75%, 80%, 85%, 90%, 95%, 96%, 97%, 98%, 99% or 100% (or any percentage in between) identical to SEQ ID NO: 99.

The PB or PBL transposase can comprise or consist of an amino acid sequence having an amino acid substitution at two or more, at three or more or at each of positions 30, 165, 282, or 538 of the sequence of SEQ ID NO: 99. The transposase can be a SPB transposase that comprises or consists of the amino acid sequence of the sequence of SEQ ID NO: 99 wherein the amino acid substitution at position 30 can be a substitution of a valine (V) for an isoleucine (I), the amino acid substitution at position 165 can be a substitution of a serine (S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 75%, 80%, 85%, 90%, 95%, 96%, 97%, 98%, 99% or 100% (or any percentage in between) identical to SEQ ID NO: 100.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 99 or SEQ ID NO: 100 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, the PB or PBL transposase is integration deficient. An integration deficient PB or PBL transposase is a transposase that can excise its corresponding transposon, but that integrates the excised transposon at a lower frequency than a corresponding wild type transposase. Examples of integration deficient PB or PBL transposases are disclosed in U.S. Pat. Nos. 6,218,185; 6,962,810, 8,399,643 and WO 2019/173636. A list of integration deficient amino acid substitutions is disclosed in U.S. Pat. No. 10,041,077.

In some aspects, the PB or PBL transposase is fused to a nuclear localization signal. Examples of PB or PBL transposases fused to a nuclear localization signal are disclosed in U.S. Pat. Nos. 6,218,185; 6,962,810, 8,399,643 and WO 2019/173636.

A transposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100X) transposase. In a preferred aspect, the Sleeping Beauty transposase comprises or consists of an amino acid sequence at least 75%, 80%, 85%, 90%, 95%, 96%, 97%, 98%, 99% or 100% (or any percentage in between) identical to SEQ ID NO: 101. In a preferred aspect, hyperactive Sleeping Beauty (SB100X) transposase comprises or consists of an amino acid sequence at least 75%, 80%, 85%, 90%, 95%, 96%, 97%, 98%, 99% or 100% (or any percentage in between) identical to SEQ ID NO: 102.

A transposon of the present disclosure can be a Helraiser transposon. An exemplary Helraiser transposon includes Helibat1, which comprises or consists of a nucleic acid sequence at least 75%, 80%, 85%, 90%, 95%, 96%, 97%, 98%, 99% or 100% (or any percentage in between) identical to SEQ ID NO: 103. In some aspects, when the transposon is a Helraiser transposon, the transposase is a Helitron transposase (for example, as disclosed in WO 2019/173636). In a preferred aspect, Helitron transposase comprises or consists of an amino acid sequence at least 75%, 80%, 85%, 90%, 95%, 96%, 97%, 98%, 99% or 100% (or any percentage in between) identical to SEQ ID NO: 104.

A transposon of the present disclosure can be a Tol2 transposon. An exemplary Tol2 transposon, including inverted repeats, subterminal sequences and the Tol2 transposase, comprises or consists of a nucleic acid sequence at least 75%, 80%, 85%, 90%, 95%, 96%, 97%, 98%, 99% or 100% (or any percentage in between) identical to SEQ ID NO: 105. In some aspects, when the transposon is a Tol2 transposon, the transposase is a Tol2 transposase (for example, as disclosed in WO 2019/173636). In a preferred aspect, Tol2 transposase comprises or consists of an amino acid sequence at least 75%, 80%, 85%, 90%, 95%, 96%, 97%, 98%, 99% or 100% (or any percentage in between) identical to SEQ ID NO: 106.

A transposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. In a preferred aspect, a TcBuster transposase comprises or consists of an amino acid sequence at least 75%, 80%, 85%, 90%, 95%, 96%, 97%, 98%, 99% or 100% (or any percentage in between) identical to SEQ ID NO: 107. The polynucleotide encoding a TcBuster transposase can comprise or consist of a naturally occurring nucleic acid sequence or a non-naturally occurring nucleic acid sequence. In a preferred aspect, a TcBuster transposase is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 108.

In some aspects, a mutant TcBuster transposase comprises one or more sequence variations when compared to a wild type TcBuster transposase as described in more detail in PCT Publication No. WO 2019/173636 and PCT/US2019/049816.

The transposon can be a nanotransposon. A nanotransposon can comprise, consist essential of, or consist of (a) a sequence encoding a transposon insert, comprising a sequence encoding a first inverted terminal repeat (ITR), a sequence encoding a second inverted terminal repeat (ITR), and an intra-ITR sequence; (b) a sequence encoding a backbone, wherein the sequence encoding the backbone comprises a sequence encoding an origin of replication having between 1 and 450 nucleotides, inclusive of the endpoints, and a sequence encoding a selectable marker having between 1 and 200 nucleotides, inclusive of the endpoints, and (c) an inter-ITR sequence. In some aspects, the inter-ITR sequence of (c) comprises the sequence of (b). In some aspects, the intra-ITR sequence of (a) comprises the sequence of (b).

The sequence encoding the backbone can comprise between 1 and 600 nucleotides, inclusive of the endpoints. In some aspects, the sequence encoding the backbone consists of between 1 and 50 nucleotides, between 50 and 100 nucleotides, between 100 and 150 nucleotides, between 150 and 200 nucleotides, between 200 and 250 nucleotides, between 250 and 300 nucleotides, between 300 and 350 nucleotides, between 350 and 400 nucleotides, between 400 and 450 nucleotides, between 450 and 500 nucleotides, between 500 and 550 nucleotides, between 550 and 600 nucleotides, each range inclusive of the endpoints.

The inter-ITR sequence can comprise between 1 and 1000 nucleotides, inclusive of the endpoints. In some aspects, the inter-ITR sequence consists of between 1 and 50 nucleotides, between 50 and 100 nucleotides, between 100 and 150 nucleotides, between 150 and 200 nucleotides, between 200 and 250 nucleotides, between 250 and 300 nucleotides, between 300 and 350 nucleotides, between 350 and 400 nucleotides, between 400 and 450 nucleotides, between 450 and 500 nucleotides, between 500 and 550 nucleotides, between 550 and 600 nucleotides, between 600 and 650 nucleotides, between 650 and 700 nucleotides, between 700 and 750 nucleotides, between 750 and 800 nucleotides, between 800 and 850 nucleotides, between 850 and 900 nucleotides, between 900 and 950 nucleotides, or between 950 and 1000 nucleotides, each range inclusive of the endpoints.

The nanotransposon can be a short nanotransposon (SNT) wherein the inter-ITR sequence comprises between 1 and 200 nucleotides, inclusive of the endpoints. The inter-ITR sequence can consist of between 1 and 10 nucleotides, between 10 and 20 nucleotides, between 20 and 30 nucleotides, between 30 and 40 nucleotides, between 40 and 50 nucleotides, between 50 and 60 nucleotides, between 60 and 70 nucleotides, between 70 and 80 nucleotides, between 80 and 90 nucleotides, or between 90 and 100 nucleotides, each range inclusive of the endpoints.

The selectable marker having between 1 and 200 nucleotides, inclusive of the endpoints, can comprise a sequence encoding a sucrose-selectable marker. The sequence encoding a sucrose-selectable marker can comprise a sequence encoding an RNA-OUT sequence. The sequence encoding an RNA-OUT sequence can comprise or consist of 137 base pairs (bp). The selectable marker having between 1 and 200 nucleotides, inclusive of the endpoints, can comprise a sequence encoding a fluorescent marker. The selectable marker having between 1 and 200 nucleotides, inclusive of the endpoints, can comprise a sequence encoding a cell surface marker.

The sequence encoding an origin of replication having between 1 and 450 nucleotides, inclusive of the endpoints, can comprise a sequence encoding a mini origin of replication. In some aspects, the sequence encoding an origin of replication having between 1 and 450 nucleotides, inclusive of the endpoints, comprises a sequence encoding an R6K origin of replication. The R6K origin of replication can comprise an R6K gamma origin of replication. The R6K origin of replication can comprise an R6K mini origin of replication. The R6K origin of replication can comprise an R6K gamma mini origin of replication. The R6K gamma mini origin of replication can comprise or consist of 281 base pairs (bp).

In some aspects of the nanotransposon, the sequence encoding the backbone does not comprise a recombination site, an excision site, a ligation site or a combination thereof. In some aspects, neither the nanotransposon nor the sequence encoding the backbone comprises a product of a recombination site, an excision site, a ligation site or a combination thereof. In some aspects, neither the nanotransposon nor the sequence encoding the backbone is derived from a recombination site, an excision site, a ligation site or a combination thereof.

In some aspects of the nanotransposon, a recombination site comprises a sequence resulting from a recombination event. In some aspects, a recombination site comprises a sequence that is a product of a recombination event. In some aspects, the recombination event comprises an activity of a recombinase (e.g., a recombinase site).

In some aspects of the nanotransposon, the sequence encoding the backbone does not further comprise a sequence encoding foreign DNA.

In some aspects of the nanotransposon, the inter-ITR sequence does not comprise a recombination site, an excision site, a ligation site or a combination thereof. In some aspects, the inter-ITR sequence does not comprise a product of a recombination event, an excision event, a ligation event or a combination thereof. In some aspects, the inter-ITR sequence is not derived from a recombination event, an excision event, a ligation event or a combination thereof. In some aspects, the inter-ITR sequence comprises a sequence encoding foreign DNA. In some aspects, the intra-ITR sequence comprises at least one sequence encoding an insulator and a sequence encoding a promoter capable of expressing an exogenous sequence in a mammalian cell. The mammalian cell can be a human cell. In some aspects, the intra-ITR sequence comprises a first sequence encoding an insulator, a sequence encoding a promoter capable of expressing an exogenous sequence in a mammalian cell and a second sequence encoding an insulator. In some aspects, the intra-ITR sequence comprises a first sequence encoding an insulator, a sequence encoding a promoter capable of expressing an exogenous sequence in a mammalian cell, a polyadenosine (polyA) sequence and a second sequence encoding an insulator. In some aspects, the intra-ITR sequence comprises a first sequence encoding an insulator, a sequence encoding a promoter capable of expressing an exogenous sequence in a mammalian cell, at least one exogenous sequence, a polyadenosine (polyA) sequence and a second sequence encoding an insulator.

Nanotransposons are described in more detail in PCT/US2019/067758.

Vector Systems

A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV). Exemplary adeno-associated viruses and recombinant adeno-associated viruses comprise two or more inverted terminal repeat (ITR) sequences located in cis next to a sequence encoding an scFv or a CAR of the disclosure. Exemplary adeno-associated viruses and recombinant adeno-associated viruses include, but are not limited to all serotypes (e.g., AAV1, AAV2, AAV3, AAV4, AAV5, AAV6, AAV7, AAV8, and AAV9). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03.

A vector of the present disclose can be a nanoparticle. Non-limiting examples of nanoparticle vectors include nucleic acids (e.g., RNA, DNA, synthetic nucleotides, modified nucleotides or any combination thereof), amino acids (L-amino acids, D-amino acids, synthetic amino acids, modified amino acids, or any combination thereof), polymers (e.g., polymersomes), micelles, lipids (e.g., liposomes), organic molecules (e.g., carbon atoms, sheets, fibers, tubes), inorganic molecules (e.g., calcium phosphate or gold) or any combination thereof. A nanoparticle vector can be passively or actively transported across a cell membrane.

The cell delivery compositions (e.g., transposons, vectors) disclosed herein can comprise a nucleic acid encoding a therapeutic protein or therapeutic agent. Examples of therapeutic proteins include those disclosed in PCT Publication No. WO 2019/173636 and PCT/US2019/049816.

Inducible Proapontotic Polypeptides

The inducible proapoptotic polypeptides disclosed herein are superior to existing inducible polypeptides because the inducible proapoptotic polypeptides of the disclosure are far less immunogenic. The inducible proapoptotic polypeptides are recombinant polypeptides, and, therefore, non-naturally occurring. Further, the sequences that are recombined to produce inducible proapoptotic polypeptides that do not comprise non-human sequences that the host human immune system could recognize as “non-self” and, consequently, induce an immune response in the subject receiving the inducible proapoptotic polypeptide, a cell comprising the inducible proapoptotic polypeptide or a composition comprising the inducible proapoptotic polypeptide or the cell comprising the inducible proapoptotic polypeptide.

The disclosure provides inducible proapoptotic polypeptides comprising a ligand binding region, a linker, and a proapoptotic peptide, wherein the inducible proapoptotic polypeptide does not comprise a non-human sequence. In certain aspects, the non-human sequence comprises a restriction site. In certain aspects, the ligand binding region can be a multimeric ligand binding region. In certain aspects, the proapoptotic peptide is a caspase polypeptide. Non-limiting examples of caspase polypeptides include caspase 1, caspase 2, caspase 3, caspase 4, caspase 5, caspase 6, caspase 7, caspase 8, caspase 9, caspase 10, caspase 11, caspase 12, and caspase 14. Preferably, the caspase polypeptide is a caspase 9 polypeptide. The caspase 9 polypeptide can be a truncated caspase 9 polypeptide. Inducible proapoptotic polypeptides can be non-naturally occurring. When the caspase is caspase 9 or a truncated caspase 9, the inducible proapoptotic polypeptides can also be referred to as an “iC9 safety switch”.

An inducible caspase polypeptide can comprise (a) a ligand binding region, (b) a linker, and (c) a caspase polypeptide, wherein the inducible proapoptotic polypeptide does not comprise a non-human sequence. In certain aspects, an inducible caspase polypeptide comprises (a) a ligand binding region, (b) a linker, and (c) a truncated caspase 9 polypeptide, wherein the inducible proapoptotic polypeptide does not comprise a non-human sequence.

The ligand binding region can comprise a FK506 binding protein 12 (FKBP12) polypeptide. The amino acid sequence of the ligand binding region that comprises a FK506 binding protein 12 (FKBP12) polypeptide can comprise a modification at position 36 of the sequence. The modification can be a substitution of valine (V) for phenylalanine (F) at position 36 (F36V). The FKBP12 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 109. The FKBP12 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 110.

The linker region can comprise, consist essential of, or consist of, the amino acid sequence at least 75%, 80%, 85%, 90%, 95%, 96%, 97%, 98%, 99% or 100% (or any percentage in between) identical to SEQ ID NO: 111 or the linker region can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 112. In some aspects, the nucleic acid sequence encoding the linker does not comprise a restriction site.

The truncated caspase 9 polypeptide can comprise an amino acid sequence that does not comprise an arginine (R) at position 87 of the sequence. Alternatively, or in addition, the truncated caspase 9 polypeptide can comprise an amino acid sequence that does not comprise an alanine (A) at position 282 the sequence. The truncated caspase 9 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 113 or the truncated caspase 9 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 114.

In certain aspects when the polypeptide comprises a truncated caspase 9 polypeptide, the inducible proapoptotic polypeptide comprises, consists essential of, or consists of, the amino acid sequence at least 75%, 80%, 85%, 90%, 95%, 96%, 97%, 98%, 99% or 100% (or any percentage in between) identical to SEQ ID NO: 115 or the inducible proapoptotic polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 116.

Inducible proapoptotic polypeptides can be expressed in a cell under the transcriptional regulation of any promoter known in the art that is capable of initiating and/or regulating the expression of an inducible proapoptotic polypeptide in that cell.

Activation of inducible proapoptotic polypeptides can be accomplished through, for example, chemically induced dimerization (CID) mediated by an induction agent to produce a conditionally controlled protein or polypeptide. Proapoptotic polypeptides not only inducible, but the induction of these polypeptides is also reversible, due to the degradation of the labile dimerizing agent or administration of a monomeric competitive inhibitor.

In certain aspects when the ligand binding region comprises a FKBP12 polypeptide having a substitution of valine (V) for phenylalanine (F) at position 36 (F36V), the induction agent can comprise AP1903, a synthetic drug (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2R*]]]]]-(9Cl) CAS Registry Number: 195514-63-7; Molecular Formula: C78H98N4O20; Molecular Weight: 1411.65)); AP20187 (CAS Registry Number: 195514-80-8 and Molecular Formula: C82H107N5O20) or an AP20187 analog, such as, for example, AP1510. As used herein, the induction agents AP20187, AP1903 and AP1510 can be used interchangeably.

Inducible proapoptotic peptides and methods of inducing these peptides are described in detail in U.S. Patent Publication No. WO 2019/0225667 and PCT Publication No. WO 2018/068022.

Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 m, preferably, about 1 m to about 5 m, and, most preferably, about 2 m to about 3 m. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In one aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

Methods of Using the Compositions of the Disclosure

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Preferably, the malignant disease is cancer. Non-limiting examples of a malignant disease or disorder include leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In preferred aspects, the treatment of a malignant disease or disorder comprises adoptive cell therapy. For example, in one aspect, the disclosure provides modified cells that express at least one disclosed antibody (e.g., scFv) and/or CAR comprising an antibody (e.g., scFv) that have been selected and/or expanded for administration to a subject in need thereof. Modified cells can be formulated for storage at any temperature including room temperature and body temperature. Modified cells can be formulated for cryopreservation and subsequent thawing. Modified cells can be formulated in a pharmaceutically acceptable carrier for direct administration to a subject from sterile packaging. Modified cells can be formulated in a pharmaceutically acceptable carrier with an indicator of cell viability and/or CAR expression level to ensure a minimal level of cell function and CAR expression. Modified cells can be formulated in a pharmaceutically acceptable carrier at a prescribed density with one or more reagents to inhibit further expansion and/or prevent cell death.

Any can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In one aspect, the administration is systemic. Systemic administration can be any means known in the art and described in detail herein. Preferably, systemic administration is by an intravenous injection or an intravenous infusion. In one aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In one example, the disclosure provides a method of treating cancer in a subject in need thereof, comprising administering to the subject a composition comprising an antibody (e.g., scFv) or a CAR comprising an antibody (e.g., scFv) the antibody or CAR specifically binds to an antigen on a tumor cell. In aspects where the composition comprises a modified cell or cell population, the cell or cell population may be autologous or allogeneic.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

Antibody/scFv Production, Screening and Purification

At least one antibody (e.g., monoclonal antibody, a chimeric antibody, a single domain antibody, a VHH, a VH, a single chain variable fragment (scFv), an antigen-binding fragment (Fab) or a Fab fragment) of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from an scFv can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, an scFv can be engineered with retention of high affinity for the antigen and other favorable biological properties. To achieve this goal, the scaffold proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate scFv to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

Screening of an scFv for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed nucleotide or peptide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. WO 91/17271, WO 91/18980, WO 91/19818, and WO 93/08278.

Other systems for generating libraries of peptides have aspects of both in vitro chemical synthesis and recombinant methods. See, PCT Patent Publication Nos. WO 92/05258, WO 92/14843, and WO 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge Antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra.

An scFv of the disclosure can bind human or other mammalian proteins with a wide range of affinities (KD). In a preferred aspect, at least one scFv of the present disclosure can optionally bind to a target protein with high affinity, for example, with a KD equal to or less than about 10⁻⁷ M, such as but not limited to, 0.1-9.9 (or any range or value therein)×10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of a scFv for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein). The measured affinity of a particular scFv-antigen interaction can vary if measured under different conditions (e.g., salt concentration, pH). Thus, measurements of affinity and other antigen-binding parameters (e.g., KD, Kon, Koff) are preferably made with standardized solutions of protein scaffold and antigen, and a standardized buffer, such as the buffer described herein.

Competitive assays can be performed with the scFv of the disclosure in order to determine what proteins, antibodies, and other antagonists compete for binding to a target protein with the scFv of the present disclosure and/or share the epitope region. These assays as readily known to those of ordinary skill in the art evaluate competition between antagonists or ligands for a limited number of binding sites on a protein. The protein and/or antibody is immobilized or insolubilized before or after the competition and the sample bound to the target protein is separated from the unbound sample, for example, by decanting (where the protein/antibody was pre-insolubilized) or by centrifuging (where the protein/antibody was precipitated after the competitive reaction). Also, the competitive binding may be determined by whether function is altered by the binding or lack of binding of the scFv to the target protein, e.g., whether the scFv molecule inhibits or potentiates the enzymatic activity of, for example, a label. ELISA and other functional assays may be used, as well known in the art.

Nucleic Acid Molecules

Nucleic acid molecules of the disclosure encoding an scFv can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified portion of at least one scFv; nucleic acid molecules comprising the coding sequence for a protein scaffold or loop region that binds to the target protein; and nucleic acid molecules which comprise a nucleotide sequence substantially different from those described above but which, due to the degeneracy of the genetic code, still encode the protein scaffold as described herein and/or as known in the art. Of course, the genetic code is well known in the art. Thus, it would be routine for one skilled in the art to generate such degenerate nucleic acid variants that code for a specific scFv of the present disclosure. See, e.g., Ausubel, et al., supra, and such nucleic acid variants are included in the present disclosure.

As indicated herein, nucleic acid molecules of the disclosure which comprise a nucleic acid encoding a scFv can include, but are not limited to, those encoding the amino acid sequence of a scFv fragment, by itself, the coding sequence for the entire protein scaffold or a portion thereof; the coding sequence for a scFv, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a protein scaffold can be fused to a marker sequence, such as a sequence encoding a peptide that facilitates purification of the fused protein scaffold comprising a protein scaffold fragment or portion.

Polynucleotides Selectively Hybridizing to a Polynucleotide as Described Herein

The disclosure provides isolated nucleic acids that hybridize under selective hybridization conditions to a polynucleotide disclosed herein. Thus, the polynucleotides can be used for isolating, detecting, and/or quantifying nucleic acids comprising such polynucleotides. For example, polynucleotides of the present disclosure can be used to identify, isolate, or amplify partial or full-length clones in a deposited library. The polynucleotides can be genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of a protein scaffold encoded by the polynucleotides described herein. The polynucleotides embrace nucleic acid sequences that can be employed for selective hybridization to a polynucleotide encoding a protein scaffold of the present disclosure. See, e.g., Ausubel, supra; Colligan, supra, each entirely incorporated herein by reference.

Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise nucleotide sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

Expression Vectors and Host Cells

The disclosure also relates to vectors that include isolated nucleic acid molecules of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DIFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

scFv Purification

An scFv can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

An scFv of the disclosure include purified products, products of chemical synthetic procedures, and products produced by recombinant techniques from a prokaryotic or eukaryotic host, including, for example, E. coli, yeast, higher plant, insect and mammalian cells. Depending upon the host employed in a recombinant production procedure, the protein scaffold of the disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A protein scaffold of the disclosure can include one or more amino acid substitutions, deletions or additions, from spontaneous or mutations and/or human manipulation, as specified herein. Amino acids in a protein scaffold of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for protein scaffold binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active protein scaffold of the disclosure. Biologically active protein scaffolds have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to protein scaffolds and fragments, as described herein, which are modified by the covalent attachment of an organic moiety. Such modification can produce a protein scaffold fragment with improved pharmacokinetic properties (e.g., increased in vivo serum half-life). The organic moiety can be a linear or branched hydrophilic polymeric group, fatty acid group, or fatty acid ester group. In particular aspect, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified protein scaffolds and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and dicarboxylic acids. A “hydrophilic polymeric group,” as the term is used herein, refers to an organic polymer that is more soluble in water than in octane. For example, polylysine is more soluble in water than in octane. Thus, a protein scaffold modified by the covalent attachment of polylysine is encompassed by the disclosure. Hydrophilic polymers suitable for modifying protein scaffolds of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein scaffold of the disclosure has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying protein scaffolds of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-A9-octadecanoate (C18, oleate), all cis-A5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified protein scaffolds and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified protein scaffolds of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified protein scaffolds and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to an aspect hereof, the term “antibody hereof” in its broadest sense also covers such analogs. Generally, in such analogs, one or more amino acid residues may have been replaced, deleted and/or added, compared to the antibodies hereof as defined herein.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). The term further includes single domain antibodies (“sdAB”) which generally refers to an antibody fragment having a single monomeric variable antibody domain, (for example, from camelids). Such antibody fragment types will be readily understood by a person having ordinary skill in the art.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In some aspects, a specific binding interaction will discriminate between desirable and undesirable antigens in a sample. In some aspects, more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | | Amino Acid |
|---|---|---|
| Non-polar (hydrophobic) | Aliphatic: | A L I V P |
| | Aromatic: | F W Y |
| | Sulfur- | M |
| | containing: | |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

### Example 1—Construction of Chimeric Stimulatory Receptors (CSRs)

Stimulation is enhanced with expression of Chimeric Stimulatory Receptors (CSRs) in the presence or absence of TCRs. In the presence of surface-expressed CSR/s, either transiently or stably expressed, enhanced primary and secondary co-stimulatory signals are delivered when T cell is treated with reagents displaying agonist mAbs. In one aspect, this schematic diagram represents an allogeneic cell. Since a fuller T-cell activation is achieved via CSR-mediated stimulatory signals, T cell activation and expansion is enhanced.

Chimeric Stimulatory Receptors (CSRs) were designed to include an antigen recognition region that comprises a CD2 extracellular domain. A panel of CSR mutants was designed within the extracellular domain of CD2. The goal of this panel was to identify mutants that no longer bind CD58 but retain their receptivity to being bound by the anti-CD2 activator reagent. This may be desirable for two main reasons: 1) CD58 expression by activated T cells may interact with the wild type (WT) CSR and possibly interfere with the optimal performance of the CSR, and 2) since the WT CSR might function as a natural ligand CAR, it is possible that T cells expressing the CSR may mediate cytotoxic activity against CD58-expressing cells, including activated T cells. Thus, a mutant CSR that cannot interact with CD58 but retains its ability to bind activating anti-CD2 reagent for optimal cell expansion is desired.

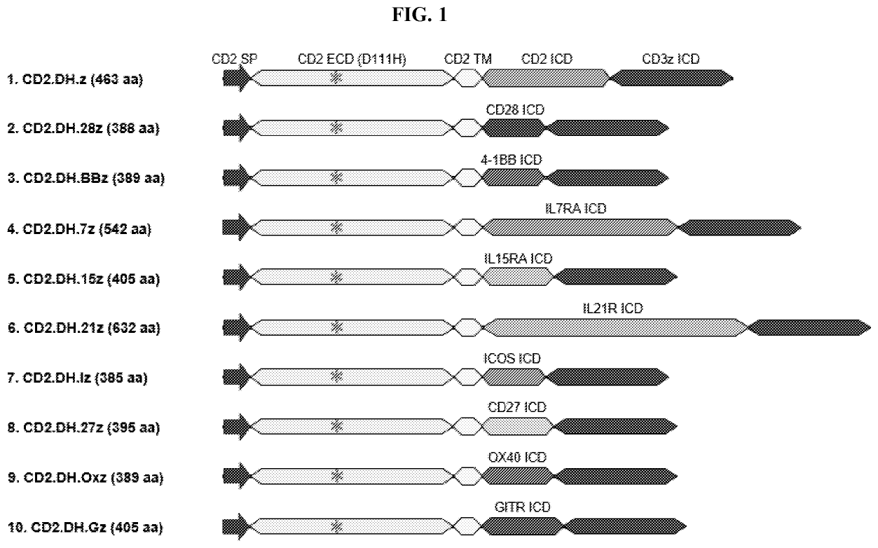

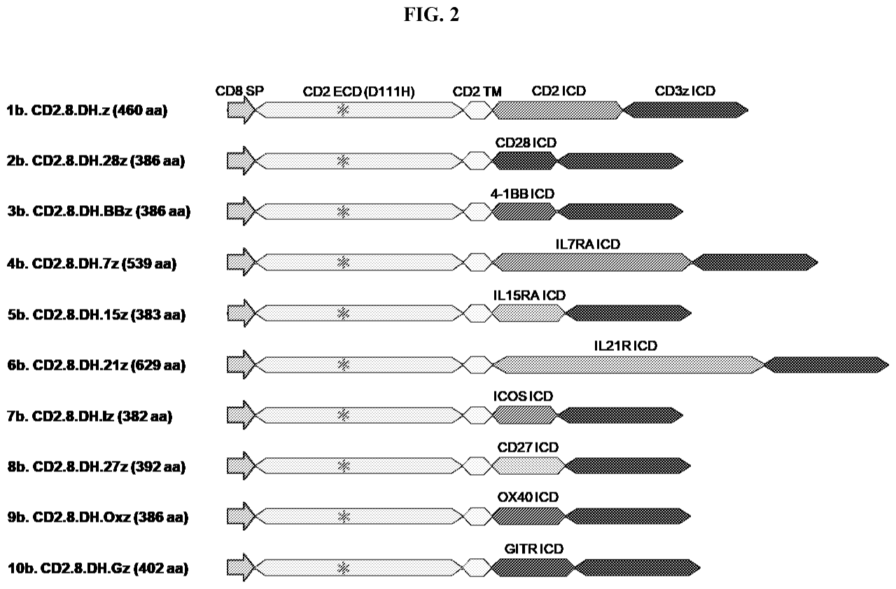

A D111H mutation within the CD2 extracellular domain (“CD2 ECD (D111H)”) retains the ability to bind to an activating CD2 reagent for optimal cell expansion and does not interact with CD58. Schematic diagrams of the CSRs of the disclosure are shown in FIG. 1 and FIG. 2. FIG. 1 shows a schematic diagram of CSRs that have a CD2 signal peptide. FIG. 2 shows a schematic diagram of CSRs that have a CD8 signal peptide. These CSRs can be used for enhanced manufacture of allogeneic or autologous CAR-T cells. CSR CD2z-D111H mutants can be delivered to allogeneic or autologous CAR-T cells during manufacturing to enhance cell growth and expansion, quality, survival, phenotype, function, subset composition, gene-editing efficiency, etc. These mutant CSRs can be delivered either transiently as encoded in mRNA or stably as encoded in a transposon.

### Example 2—Functional Characterization of Chimeric Stimulatory Receptors

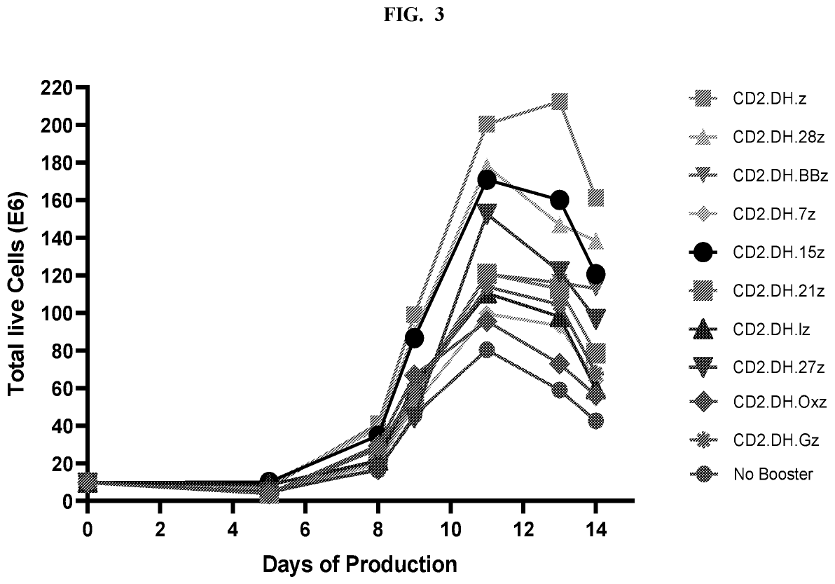

To test the effects of the CSRs on CAR-T cell expansion, Pan T cells isolated from normal donor blood were genetically modified using the piggyBac® DNA modification system in combination with the Cas-CLOVER™ gene-editing system. Cells were electroporated in a single reaction with a transposon encoding at least a CAR and a selection gene, an mRNA encoding a CSR, an mRNA encoding the super piggyBac® transposase enzyme, an mRNA encoding Cas-CLOVER™, and multiple guide RNA (gRNA) targeting TCRb and b2M in order to knockout the TCR and MHCI (double-knockout; DKO). The cells were subsequently stimulated with agonist mAbs anti-CD2, anti-CD3 and anti-CD28, and were later selected for genetic modification over the course of a 14-day culture period. At the end of the initial culture period all T cells expressed the CAR, indicating successful selection for genetically-modified cells. A greater degree of expansion of the DKO cells was observed for cells expressing CSRs in comparison to cells that did not express a CSR (No Booster) (FIG. 3). Thus, expression of CSR enhances CAR-T cell expansion during production.

The effect of CSR expression on the memory phenotype of DKO CAR-T cells was tested. DKO CAR-T cells produced with CSR or without a CSR (No Booster) were stained for expression of surface CD45RA, CD45RO, and CD62L to define Tscm, Tcm, Tem, and Teff cells; Tscm (CD45RA+CD45RO−CD62L+), Tcm (CD45RA−CD45RO+CD62L+), Tem (CD45RA−CD45RO+CD62L−), Teff (CD45RA+CD45RO−CD62L−). The proportion of Teff, Tscm, Tcm and Tem for DKO CAR-T cells with or without CSRs are shown in Tables 1 and 2. DKO CAR-T cells either with or without a CSR are comprised predominantly of exceptionally high levels of favorable Tscm and Tcm cells. Thus, memory phenotype of DKO CAR-T is not significantly affected by CSR co-expression.

TABLE 1
Proportion of Teff, Tscm, Tcm and Tem in CD8+ DKO CAR-T cells
with or without expression of a chimeric stimulatory receptor

| CSR | Teff | Tscm | Tcm | Tem |
|---|---|---|---|---|
| CD2.DH.z | 4.9e−002 | 41.2 | 52.4 | 0.3 |
| CD2.DH.28z | 0.18 | 40.6 | 54.5 | 0.5 |
| CD2.DH.BBZ | 0.34 | 47.2 | 46.7 | 0.75 |
| CD2.DH.7z | 0.42 | 43.4 | 50.9 | 0.94 |
| CD2.DH.15z | 0.10 | 47.4 | 47.5 | 0.29 |
| CD2.DH.21z | 0.3 | 46.2 | 48.2 | 0.61 |
| CD2.DH.1z | 0.41 | 48 | 46.7 | 1.08 |
| CD2.DH.27z | 0.2 | 35.8 | 59.2 | 0.45 |
| CD2.DH.Oxz | 0.35 | 46.5 | 48.5 | 0.7 |
| CD2.DH.Gz | 0.48 | 44.8 | 49.9 | 0.76 |
| No Booster | 0.65 | 39.6 | 54.7 | 1.59 |

TABLE 2
Proportion of Teff, Tscm, Tern and Tem in CD4+ DKO CAR-T cells
with or without expression of a chimeric stimulatory receptor

| CSR | Teff | Tscm | Tcm | Tem |
|---|---|---|---|---|
| CD2.DH.z | 0.43 | 7.68 | 84 | 6.97 |
| CD2.DH.28z | 1.03 | 8.8 | 77.9 | 11.8 |
| CD2.DH.BBZ | 1.81 | 11.4 | 72.2 | 13.9 |
| CD2.DH.7z | 1.42 | 9.58 | 72.8 | 15.7 |
| CD2.DH.15z | 0.67 | 9.33 | 80.2 | 9.09 |
| CD2.DH.21z | 1.1 | 13.4 | 71.4 | 13.1 |
| CD2.DH.1z | 1.77 | 8.77 | 70.1 | 18.8 |
| CD2.DH.27z | 0.99 | 8.97 | 78.3 | 11.1 |
| CD2.DH.Oxz | 1.45 | 9.97 | 72.6 | 15.6 |
| CD2.DH.Gz | 1.44 | 9.64 | 73.8 | 14.7 |
| No booster | 1.25 | 7.88 | 75.5 | 15 |

### Example 3—In Vivo Effects of CAR-T Cells Expressing Chimeric Stimulatory Receptors (CSRs)

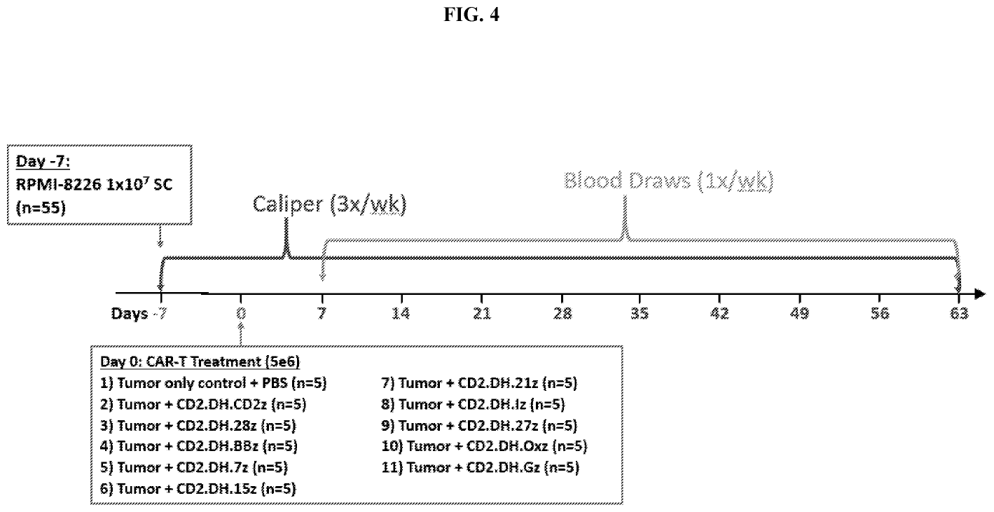

To test the effects of the allogeneic CAR-T cells expressing different CSRs described in Example 2 on anti-tumor efficacy, an in vivo experiment was conducted using a murine xenograft model of multiple myeloma. A schematic of the experimental procedure is depicted in FIG. 4. and 10 different allogeneic CAR-T cells each produced with the 10 different CSRs. Specifically, the RPMI-8226 cell line was injected subcutaneously into female NSG mice at a dose of 1×10⁷ cells (Day −7) and later treated with allo CAR-T cells by intravenous (IV) injection at a ‘stress’ dose (5×10⁶) on Day 0 when tumors were established (75-125 mm³ [target mean ˜100 mm³] by caliper measurement). The ‘stress’ dose was used for greater resolution in detecting possible functional differences in efficacy among the CAR-T cells produced with the different CSRs. Treatment with PBS was used as a negative control. An allogeneic CAR-T cell expressing CD2.DHz was used as a positive control.

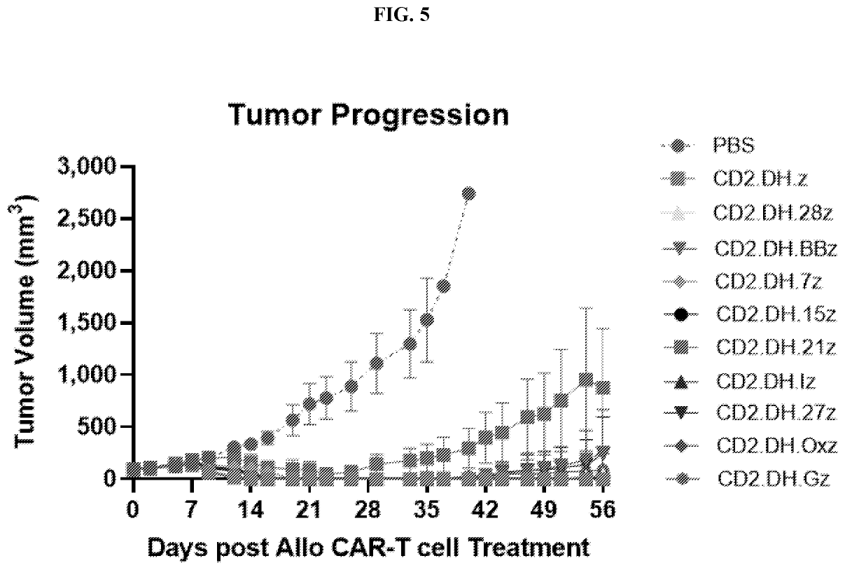

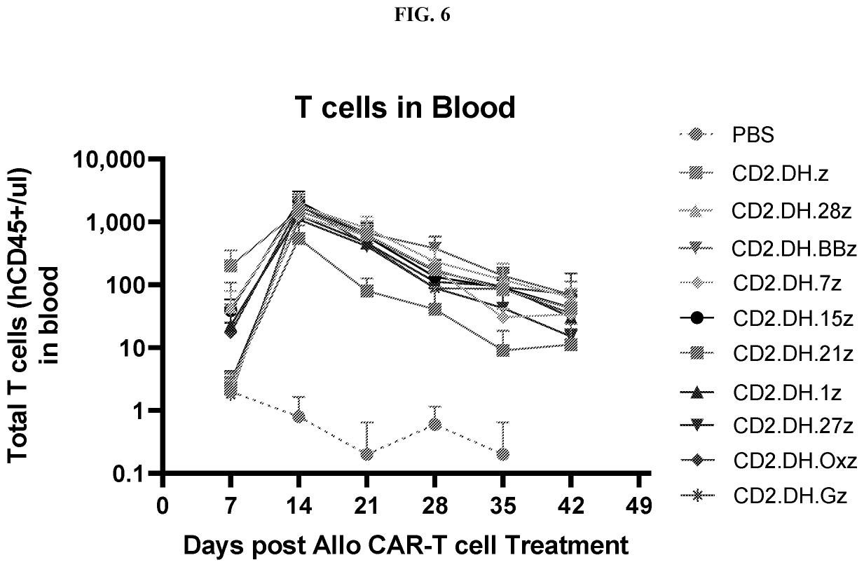

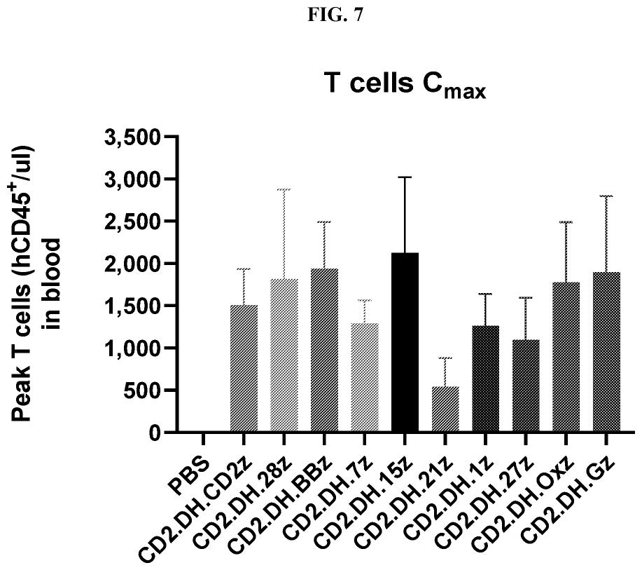

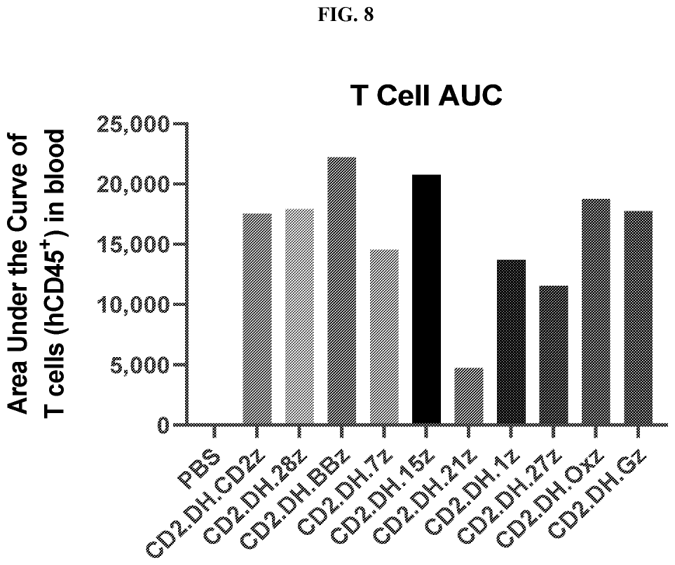

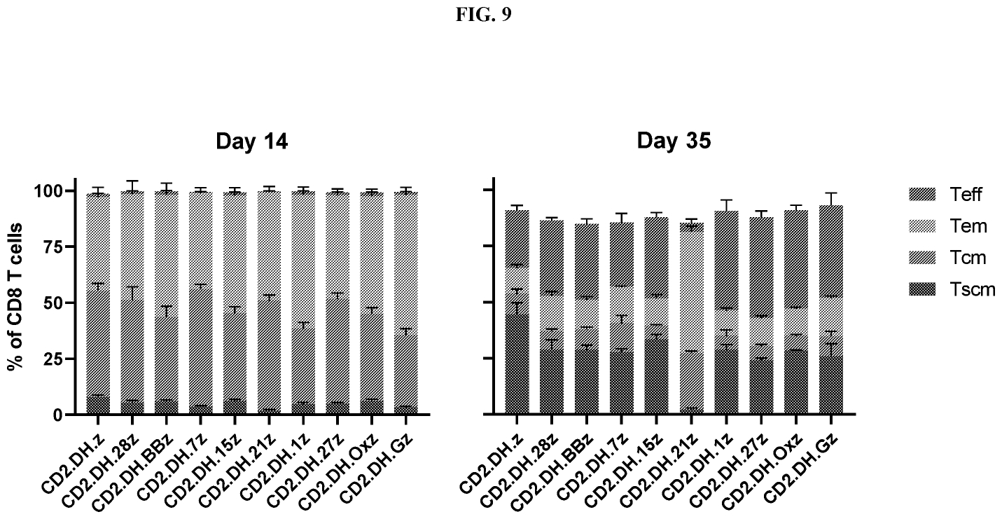

The results of this experiment are shown in FIGS. 5-9. Treatment of the animals with CAR-T cells expressing various CSRs of the disclosure resulted in a decrease in tumor volume in comparison to the PBS control (FIG. 5). The tumor size following 56 days post treatment with allogeneic CAR-T cells expressing the CSRs was comparable to that of the positive control. The total T cells in blood was quantified following treatment with the allogeneic CAR-T cells expressing CSRs (FIG. 6). The peak level of T cells in blood was also quantified following treatment with CAR-T cells expressing CSRs and displayed as a group average over all animals (FIG. 7). All animals displayed an increase in peak level T cells relative to the PBS negative control. CAR-T cells expressing CD2.DH28z, CD2.DH.BBz, CD2.DH.15z, CD2.DH.Oxz and CD2.DH.Gz displayed an increase in the peak level of T cells in blood relative to the CD2.DH.CD2z (also known as CD2.DH.z) control. The T cell Area under the curve (T cell AUC) was calculated following treatment with each allogeneic CAR-T cell (FIG. 8 and Table 3). All animals displayed an increase in T cell AUC relative to the PBS negative control. CAR-T cells expressing CD2.DH28z, CD2.DH.BBz, CD2.DH.15z, CD2.DH.Oxz and CD2.DH.Gz displayed an increase in the peak level of T cells in blood relative to the CD2.DH.CD2z (also known as CD2.DH.z) control. The effects on the proportion of Teff, Tem, Tcm and Tscm cells following treatment with each allogeneic CAR-T cell expressing various CSRs was comparable to treatment with a positive control (CAR-T cell expressing a CD2.DH.z CSR).

TABLE 3
Area under the curve of T cell (hCD45+) in Blood following treatment
with allogeneic CAR-T cells

| CSR expressed on CAR-T cell | T cell Area under the curve (AUC) |
|---|---|
| PBS negative control | 19 |
| CD2.DH.z | 17,535 |
| CD2.DH.28z | 17,898 |
| CD2.DH.BBz | 22,213 |
| CD2.DH.7z | 14,544 |
| CD2.DH.15z | 20,789 |
| CD2.DH.21z | 4,745 |
| CD2.DH.1z | 13,701 |
| CD2.DH.27z | 11,542 |
| CD2.DH. Oxz | 18,754 |
| CD2.DH.Gz | 17,748 |

1. A non-naturally occurring chimeric stimulatory receptor (CSR) comprising:
(a) an ectodomain comprising a signal peptide and an activation component, wherein the signal peptide comprises a CD2 signal peptide or a CD8a signal peptide, and wherein the activation component comprises a CD2 extracellular domain or a portion thereof to which an agonist binds;
(b) a transmembrane domain, wherein the transmembrane domain comprises a CD2 transmembrane domain or a portion thereof, and
(c) an endodomain comprising a cytoplasmic domain and a signal transduction domain, wherein the cytoplasmic domain is a CD2 intracellular domain, a CD28 intracellular domain, a 4-1BB intracellular domain, an IL17RA intracellular domain, an IL15RA intracellular domain, an IL21R intracellular domain, an ICOS intracellular domain, a CD27 intracellular domain, an OX40 intracellular domain or a GITR intracellular domain or any combination thereof, and wherein the signal transduction domain comprises a CD3ζ protein or a portion thereof, and
wherein the signal peptide and the cytoplasmic domain are not derived from the same protein.

2. The CSR of claim 1, wherein the signal peptide comprises a CD2 signal peptide.

3. The CSR of claim 1, wherein the CD2 signal peptide comprises an amino acid sequence of SEQ ID NO: 5.

4. The CSR of claim 1, wherein the signal peptide comprises a CD8a signal peptide.

5. The CSR of claim 1, wherein the CD8α signal peptide comprises an amino acid sequence of SEQ ID NO: 7.

6. The CSR of claim 1, wherein the activation component comprises a modification.

7. The CSR of claim 1, wherein the modification comprises a mutation or a truncation of the amino acid sequence of the CD2 extracellular domain or a portion thereof to which an agonist binds in comparison to the wildtype sequence of the CD2 extracellular domain or portion thereof.

8. The CSR of claim 7, wherein the CSR comprising a mutation or truncation of a CD2 extracellular domain or a portion thereof to which an agonist binds does not bind CD58.

9. The CSR of claim 7, wherein the CD2 extracellular domain or portion thereof comprising the mutation or truncation comprises an amino acid sequence of SEQ ID NO: 3.

10. The CSR of claim 1, wherein the CD2 transmembrane domain or a portion thereof comprises an amino acid sequence of SEQ ID NO: 9.

11. The CSR of claim 1, wherein the CD2 intracellular domain comprises an amino acid sequence of SEQ ID NO: 13.

12. The CSR of claim 1, wherein the CD28 intracellular domain comprises an amino acid sequence of SEQ ID NO: 15.

13. The CSR of claim 1, wherein the 4-1BB intracellular domain comprises an amino acid sequence of SEQ ID NO: 17.

14. The CSR of claim 1, wherein the IL17RA intracellular domain comprises an amino acid sequence of SEQ ID NO: 19.

15. The CSR of claim 1, wherein the IL15RA intracellular domain comprises an amino acid sequence of SEQ ID NO: 21.

16. The CSR of claim 1, wherein the IL21R intracellular domain comprises an amino acid sequence of SEQ ID NO: 23.

17. The CSR of claim 1, wherein the ICOS intracellular domain comprises an amino acid sequence of SEQ ID NO: 25.

18. The CSR of claim 1, wherein the CD27 intracellular domain comprises an amino acid sequence of SEQ ID NO: 27.

19. The CSR of claim 1, wherein the OX40 intracellular domain comprises an amino acid sequence of SEQ ID NO: 29.

20. The CSR of claim 1, wherein the GITR intracellular domain comprises an amino acid sequence of SEQ ID NO: 31.

21. The CSR of claim 1, wherein the signal transduction domain comprising a CD3ζ protein or a portion thereof comprises an amino acid sequence of SEQ ID NO: 11.

22. The CSR of claim 1, wherein the CSR comprises an amino acid sequence of SEQ ID NO: 39.

23. The CSR of claim 1, wherein the CSR comprises an amino acid sequence of SEQ ID NO: 43.

24. The CSR of claim 1, wherein the CSR comprises an amino acid sequence of SEQ ID NO: 47.

25. The CSR of claim 1, wherein the CSR comprises an amino acid sequence of SEQ ID NO: 51.

26. The CSR of claim 1, wherein the CSR comprises an amino acid sequence of SEQ ID NO: 55.

27. The CSR of claim 1, wherein the CSR comprises an amino acid sequence of SEQ ID NO: 59.

28. The CSR of claim 1, wherein the CSR comprises an amino acid sequence of SEQ ID NO: 63.

29. The CSR of claim 1, wherein the CSR comprises an amino acid sequence of SEQ ID NO: 67.

30. The CSR of claim 1, wherein the CSR comprises an amino acid sequence of SEQ ID NO: 71.

31. The CSR of claim 1, wherein the CSR comprises an amino acid sequence of SEQ ID NO: 37.

32. The CSR of claim 1, wherein the CSR comprises an amino acid sequence of SEQ ID NO: 41.

33. The CSR of claim 1, wherein the CSR comprises an amino acid sequence of SEQ ID NO: 45.

34. The CSR of claim 1, wherein the CSR comprises an amino acid sequence of SEQ ID NO: 49.

35. The CSR of claim 1, wherein the CSR comprises an amino acid sequence of SEQ ID NO: 53.

36. The CSR of claim 1, wherein the CSR comprises an amino acid sequence of SEQ ID NO: 57.

37. The CSR of claim 1, wherein the CSR comprises an amino acid sequence of SEQ ID NO: 61.

38. The CSR of claim 1, wherein the CSR comprises an amino acid sequence of SEQ ID NO: 65.

39. The CSR of claim 1, wherein the CSR comprises an amino acid sequence of SEQ ID NO: 69.

40. The CSR of claim 1, wherein the CSR comprises an amino acid sequence of SEQ ID NO: 73.

41. A nucleic acid sequence encoding the CSR of claim 1.
